(19) United States
(12) Patent Application Publication
(10) Pub. No.: US 2025/0222646 A1
(43) Pub. Date: Jul. 10, 2025

(54) INTRAORAL MONITOR AND METHODS OF MANUFACTURE

(71) Applicant: UCHU Biosensors, Inc., Cambridge, MA (US)

(72) Inventors: Daniel Weinstein, Lincoln, MA (US); Noah William Hill, Tacoma, WA (US); Saam Bozorg, West Hartford, CT (US)

(21) Appl. No.: 18/843,876

(22) PCT Filed: Mar. 3, 2023
(86) PCT No.: PCT/US2023/063706

§ 371 Date: Sep. 4, 2024

Related U.S. Application Data
(60) Provisional application No. 63/316,639, filed on Mar. 4, 2022.

(51) Int. Cl. B29C 51/26; A61C 7/08; B29L 31/00

(52) U.S. Cl. CPC B29C 51/268; A61C 7/08; B29L 2031/753

(57) ABSTRACT

A method, for forming an intraoral monitor includes forming a first portion of a retainer corresponding in shape to teeth. The method includes forming a second portion of a retainer corresponding in shape to the teeth and including a pocket for insertion of a sensor. The method includes coupling the first and second portions of the retainer to envelop the sensor in the pocket, the retainer having an opening such that the sensor is exposed to the buccal side of the mouth of a user when the retainer is mounted on the teeth.

### CROSS-REFERENCE TO RELATED APPLICATIONS

This application is a Patent Cooperation Treaty application that claims the benefit of priority to U.S. Provisional Patent Application 63/316,639 filed on Mar. 4, 2022, titled “INTRAORAL MONITOR AND METHODS OF MANUFACTURE,” the entirety of which is hereby incorporated reference herein.

### BACKGROUND

Embodiments of the present disclosure generally relate to the manufacture of electronic devices and fixtures therefor, for use within the oral cavity to measure biological or chemical variables, including pH, temperature or analyte concentrations, such as to wirelessly transmit the measurements to a separate device.

### BRIEF SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter includes a method for forming an intraoral monitor, the method including, providing a first base mold corresponding in shape to at least a portion of a set of teeth, thermoforming an inner retainer around the first base mold, the inner retainer configured to abut at least a portion of a buccal side of the set of teeth, providing a second base model, the second base model corresponding to the set of teeth and the sensor, thermoforming an outer retainer around the second base model, the outer retainer configured to mount at least a portion of the set of teeth wherein, the outer retainer includes, an inner surface abutting the set of teeth and an outer surface. The outer surface includes occlusal side, a buccal side, a lingual side and a pocket disposed on the buccal side, the pocket having an opening formed on the inner surface. The method includes positioning a sensor in the pocket, coupling the outer retainer to the inner retainer, and removing a portion of the outer retainer proximate the sensor to form an opening in the outer retainer.

In some embodiments, forming the inner retainer comprises trimming a perimeter portion of the inner retainer.

In some embodiments, forming the outer retainer comprises trimming a perimeter of the outer retainer.

In some embodiments, positioning the sensor in the pocket comprises applying an adhesive to the sensor.

In some embodiments, positioning the sensor in the pocket further comprises applying an adhesive to an exposed inner side of after the sensor is positioned in the pocket.

In some embodiments, coupling the outer retainer to the inner retainer includes curing the adhesive via exposure to ultraviolet light.

In some embodiments, removing a portion of the outer retainer proximate the sensor includes cutting a portion of the outer retainer away from the sensor, thereby exposing a transducer disposed within the sensor.

In some embodiments, the method further includes applying an adhesive to a seam formed between the outer retainer and the sensor.

In some embodiments, coupling the outer retainer to the inner retainer comprises exposing an overlapping area formed by the outer retainer and the inner retainer to a laser beam.

In some embodiments, the sensor is positioned adjacent to a single tooth.

To achieve these and other advantages and in accordance with the purpose of the disclosed subject matter, as embodied and broadly described, the disclosed subject matter includes a method for forming an intraoral monitor includes providing an inner thermoform mold corresponding in shape to at least a portion of a set of teeth, providing an outer thermoform mold corresponding in shape to at least a portion of the set of teeth and the contours of a sensor protrusion formed thereon, thermoforming an inner retainer around the inner thermoform mold, the inner retainer configured to mount at least a portion of the set of teeth wherein the inner retainer includes an inner surface configured to abut the set of teeth and an outer surface. The method further includes thermoforming an outer retainer around the outer thermoform mold, the outer retainer having a pocket formed by the protrusion, positioning a sensor in the pocket, applying an adhesive to an exposed portion of the sensor once positioned in the pocket, coupling the outer retainer and sensor to the inner retainer, thereby closing the pocket, and removing a portion of the outer retainer proximate the sensor to form an opening in the outer retainer.

In some embodiments, forming the inner retainer comprises trimming a perimeter portion of the inner retainer.

In some embodiments, forming the outer retainer comprises trimming a perimeter of the outer retainer.

In some embodiments, positioning the sensor in the pocket comprises applying an adhesive to the sensor.

In some embodiments, coupling the outer retainer to the inner retainer comprises curing the adhesive via exposure to ultraviolet light.

In some embodiments, removing a portion of the outer retainer proximate the sensor comprises cutting a portion of the outer retainer away from the sensor, thereby exposing a transducer disposed within the sensor.

In some embodiments, the method further includes applying an adhesive to a seam formed between the outer retainer and the sensor.

In some embodiments, coupling the outer retainer to the inner retainer comprises exposing an overlapping area formed by the outer retainer and the inner retainer to a laser beam.

In some embodiments, the sensor is positioned adjacent to a single tooth.

To achieve these and other advantages and in accordance with the purpose of the disclosed subject matter, as embodied and broadly described, the disclosed subject matter includes a method for forming an intraoral monitor, the method including providing a contour model, the contour model defining an outer mold line of at least a portion of a set of teeth, additively forming the retainer based on the contour model, the retainer configured to mount onto at least a portion of the set of teeth, the retainer comprising an inner surface configured to abut at least a portion of the set of teeth and an outer surface, the outer surface including a buccal side, an occlusal side and a lingual side, wherein forming the retainer comprises forming a sensor recess coextensive with the outer surface. The method includes forming a sensor shell, the sensor shell including a mounting side configured to mount on the sensor recess, the sensor shell further having an opening disposed opposite the mounting side. The method includes applying an adhesive to a sensor and the sensor shell, coupling the sensor within the sensor shell, the sensor comprising a transducer side proximate the opening in the sensor shell and coupling the mounting side of the sensor shell to the sensor recess.

In some embodiments, the sensor shell includes a top wall, a bottom wall, left wall and right wall, each disposed perpendicularly to the mounting side.

In some embodiments, the sensor recess includes a top wall, a bottom wall, a left wall and a right wall corresponding with the sensor shell.

In some embodiments, applying an adhesive to the sensor recess includes applying an adhesive to the top, bottom, left and right walls.

In some embodiments, coupling the sensor to the sensor shell comprises curing the adhesive via exposure to ultraviolet light.

In some embodiments, coupling the sensor shell to the retainer comprises curing the adhesive via exposure to ultraviolet light.

In some embodiments, the method further includes applying an adhesive to a seam formed between the sensor shell and the sensor.

In some embodiments, the method further includes subjecting the retainer and retainer shell to ultraviolet light.

In some embodiments, the sensor is positioned adjacent to a single tooth.

To achieve these and other advantages and in accordance with the purpose of the disclosed subject matter, as embodied and broadly described, the disclosed subject matter includes method for forming an intraoral monitor, the method including providing a sensor, forming a sensor shell, the sensor shell configured to mount onto the sensor and wherein the sensor shell further includes a mounting side, a transducer opening side and at least one sidewall therebetween. The method includes applying an adhesive to the sensor and/or the at least one sidewall, coupling the sensor to the sensor shell, applying an adhesive to a seam formed by the sensor and/or the transducer opening side of the sensor shell. The method includes providing a fixture plate, the fixture plate having a sensor shell recess disposed thereon and configured to receive the sensor shell and an ejector pin having a first end disposed underneath the sensor shell recess and a second end disposed above the fixture plate, defining a member therebetween. The method includes additively forming a retainer on the fixture the plate, the retainer formed in alignment with the sensor shell and sensor and attached to the sensor shell.

In some embodiments, the sensor is positioned adjacent to a single tooth.

In some embodiments, forming the retainer comprises forming the retainer coextensively with the sensor shell.

In some embodiments, the method includes removing the retainer from the fixture plate by moving the ejector pin to contact the sensor shell.

In some embodiments, the fixture plate is removably attached to a build plate within a 3D printer.

In some embodiments, the sensor shell is formed from the same material as the retainer.

In some embodiments, the retainer and sensor shell are subjected to ultraviolet light.

In some embodiments, the sensor comprises at least two planar surfaces, each planar surface adjoining an adjacent planar surface at a rounded edge portion.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 is a method for forming an intraoral monitor according to an aspect of the disclosed subject matter.

FIGS. 2A-2B are exemplary illustration of a scan of a human's mouth.

FIGS. 2C-2D are exemplary illustrations of retainer model with the contours of the human's teeth and gums.

FIGS. 3A-3C are exemplary illustrations of a scan and retainer model with a sensor model and occlusal model overlaid thereon.

FIGS. 4A-4C are exemplary illustrations of a of a sensor model with location features and spacing features overlaid thereon.

FIG. 4D is exemplary illustrations of a scan, retainer model and sensor model.

FIGS. 5A-5B are exemplary illustrations of a sensor model with a cutout model attached thereto.

FIGS. 6A-6C are exemplary illustrations of a scan with a sensor model attached thereto with a cutout therein from the cutout model.

FIGS. 7A-7B are illustrations of a scan of human teeth with a sensor model attached thereto.

FIGS. 7C-7D are illustrations of a scan, a retainer model thereon with sensor contour therein, and an occlusal model of the upper teeth, in accordance with an aspect of the disclosed subject matter.

FIGS. 8A-8D are representations of the steps of cutting a portion of a retainer model according to the cutout model.

FIGS. 9A-9C are representations of a scan of human teeth and the sensor with an inner retainer model placed thereon.

FIGS. 10A-10C are representations of a cutout of a scan of human teeth and the inner retainer model.

FIGS. 11A-11E are representations of a model of human teeth and sensor with the model of the inner retainer placed thereon.

FIGS. 12A-12B are representations of a mold formed from the model of human teeth, sensor and inner retainer.

FIG. 13 is a representation of an inner retainer model in accordance with the disclosed subject matter.

FIG. 14 is a representation of a mold formed from the model of human teeth, sensor and inner retainer in a thermoforming machine.

FIGS. 15A-15B are representations of molds placed on a thermoforming machine with plastic formed thereon.

FIG. 16 is a representation of a trimming procedure using a rotating power tool.

FIG. 17 are representations of thermoformed retainer formed on the mold after trimming.

FIG. 18A-18B are representations of outer retainer formed over a mold, showing the contours of the humans teeth and sensor and inner retainer formed therein.

FIG. 19A-19C are representations of an outer retainer with the contours of human teeth, sensor and inner retainer.

FIG. 20A-20C are illustrations of a sensor and application of an adhesive on portions thereof.

FIG. 21 is a representation of a sensor placed in the pocket formed in the outer retainer as viewed from the inner surface.

FIG. 22 is a representation of a sensor placed in the pocket of the outer retainer and an adhesive being applied to said sensor.

FIG. 23A-23B is a mold having the contours of human teeth and an inner retainer, wherein the inner retainer is placed on the mold and adhesive is cured thereon by UV light.

FIG. 24A-24B are representations of curing adhesive between the retainers and the sensor by exposure to UV light.

FIG. 25A are representations of removing a portion of the outer retainer proximate the sensor using a rotary power tool.

FIG. 25B are representations of the outer retainer showing the removed portion thereof proximate the sensor and transducer.

FIG. 26A are representations of the outer retainer with portion removed and seam created by the sensor and retainers

FIG. 26B are representations of the seam formed between the sensor and the outer retainer and application of adhesive thereto.

FIGS. 27A-27B are representations of overlapping portions of the inner retainer and outer retainer and an outline placed thereon for coupling said retainers as viewed from inner surface and outer surface, respectively.

FIG. 28 is a method for forming an intraoral monitor according to an aspect of the disclosed subject matter.

FIGS. 29A-29B are exemplary illustration of a scan of a human's teeth and gums and a retainer model with the contours of the human's teeth and gums.

FIGS. 30A-30C are exemplary illustrations of a scan and retainer model with a sensor model and occlusal model overlaid thereon.

FIGS. 31A-31C are exemplary illustrations of a of a sensor model with location features and spacing features overlaid thereon.

FIG. 31D is exemplary illustrations of a scan, retainer model and sensor model.

FIGS. 32A-32B are exemplary illustrations of a sensor model with a cutout model attached thereto.

FIGS. 33A-33B are illustrations of a model of human teeth with a sensor model attached thereto.

FIGS. 34A-34B are illustrations of a model of teeth and gums, a retainer model thereon having sensor contours, and an occlusal model of the upper teeth, in accordance with an aspect of the disclosed subject matter.

FIGS. 35A-35C are representations of the steps of cutting a portion of a retainer model according to the cutout model.

FIGS. 36A-36C are exemplary illustrations of a scan with a sensor model attached thereto with a cutout therein from the cutout model.

FIG. 37 is a representation of an outer retainer having the contours of human teeth and gums and a sensor located thereon.

FIG. 38 depicts various views of an outer retainer mold formed from the model of human teeth and sensor.

FIG. 39 depicts a mold formed with the contours of a human's teeth and gums.

FIGS. 40A-40B are representations of inner retainer and outer retainer molds placed in a thermoforming machine in accordance with the aspect of the disclosed subject matter.

FIG. 41 depicts a thermoformed retainer formed on the mold.

FIGS. 42A-42C depicts representations of an outer retainer with the contours of human teeth and sensor with a cutaway block shown removed therefrom.

FIGS. 43A-43C are illustrations of a sensor and application of an adhesive on portions thereof.

FIGS. 44A-44B are representations of a sensor placed in the pocket formed in the outer retainer.

FIG. 45 depicts an inner retainer thermoformed around a mold and an outer retainer containing a sensor being coupled together.

FIG. 46 is a representation of adhesive being cured by exposure to UV light, thereby coupling the outer retainer with the sensor placed in the pocket and an inner retainer.

FIG. 47 depicts representations of removing a portion of the outer retainer proximate the sensor using a rotary power tool.

FIG. 48 are representations of the outer retainer showing the removed portion thereof proximate the sensor and transducer.

FIGS. 49A-49C are representations of the outer retainer with portion removed and seam created by the sensor and the outer retainer and application of adhesive to the seam.

FIGS. 50A-50B are representations of overlapping portions of the inner retainer and outer retainer and an outline placed thereon for coupling said retainers as viewed from inner surface and outer surface, respectively.

FIG. 51 depicts a method for forming an intraoral monitor in accordance with the disclosed subject matter.

FIGS. 52A-52C are exemplary illustration of a scan of a human's teeth and gums and a retainer model with the contours of the human's teeth and gums.

FIGS. 53A-53B are exemplary illustrations of a scan and retainer model with a sensor model and occlusal model overlaid thereon.

FIG. 54 depicts exemplary illustrations of a of a sensor model with location features and spacing features overlaid thereon.

FIG. 55 is exemplary illustrations of a scan, retainer model and sensor model.

FIG. 56 depicts exemplary illustrations of a model of human teeth and a sensor.

FIGS. 57A-57B depict exemplary steps of the cutout process of the retainer model.

FIGS. 58A-58B are illustrations of a model of human teeth with a sensor model attached thereto.

FIGS. 59A-59C are illustrations of a model of teeth and gums and a retainer model thereon having the sensor contours, in accordance with an aspect of the disclosed subject matter.

FIGS. 60A-60B depict exemplary steps for cutting a portion of the pocket of the retainer proximate the sensor according to a cutout model.

FIG. 61A-61B depict representations of a retainer model having the contours of human teeth and gums and a sensor located thereon, wherein the sensor model has an opening from the cut.

FIG. 62A-62C depict representations of a retainer model having the contours of human teeth and gums and a sensor located thereon, wherein the sensor model has an opening from the cut and a portion of the retainer proximate the pocket circumscribed.

FIG. 63A-63B depict various views of a cut outline and a cut operation of the retainer proximate the sensor pocket.

FIG. 64A depicts various views of a model of a sensor shell after the cutout operation.

FIG. 64B depicts various views of a retainer model after the cutout operation, the retainer model having a sensor recess.

FIGS. 65A-65B depict representations of a 3D printed retainer and 3D printed sensor shell according to the models.

FIG. 66 depicts representations of a 3D printed retainer and 3D printed sensor shell according to the models.

FIG. 67 depicts a sensor and application of an adhesive on portions thereof.

FIG. 68 depicts various views of a sensor placed in the sensor shell, wherein the sensor is fully seated flush in the sensor shell.

FIG. 69 depicts views of the retainer and sensor recess with the application of an adhesive in the sensor recess.

FIG. 70 depicts views of the retainer coupled to the sensor shell having the sensor disposed therein.

FIG. 71 depicts views of the retainer and sensor shell being subjected to UV light to cure adhesive.

FIG. 72 depicts views of the sensor shell and retainer.

FIG. 73 depicts representations of the sensor and sensor shell forming a seam therebetween and adhesive applied to the seam.

FIG. 74 depicts the retainer being subject to UV light.

FIG. 75 the full retainer having the sensor shell and sensor coupled thereto.

FIGS. 76-77 depict the retainer mounted on a model of teeth and gums and an occlusal model placed there over to check sensor location on the retainer.

FIG. 78 is a representation of a method for forming an intraoral monitor in accordance with another aspect of the disclosed subject matter.

FIG. 79 depicts a sensor and application of an adhesive on portions thereof.

FIG. 80 depicts various views of a sensor placed in the sensor shell, wherein the sensor is fully seated flush in the sensor shell.

FIG. 81 depicts representations of the sensor and sensor shell having a seam formed therebetween and adhesive applied to the seam.

FIGS. 82A-82D depict views of a fixture plate in accordance with an aspect of the disclosed subject matter.

FIG. 83 depicts views of a 3D printed retainer printed on the fixture plate.

FIG. 84 depicts a fixture plate having a sensor shell recess formed therein and an injector pin.

FIG. 85 depicts the retainer being subjected to UV light.

FIG. 86A-86B depict various views of and an embodiment of a sensor within the sensor shell and an embodiment of the sensor having contours suitable for sensor shell placement.

FIGS. 87A-87B depict various views of a retainer having a sensor attached thereto, the retainer configured to mount on only 1-3 teeth.

### DETAILED DESCRIPTION OF EXAMPLARY EMBODIMENTS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

As used herein, when a quantifiable parameter is described as having a value “between” a first value X and a second value Y, it shall include the parameter having a value of: at least X, no more than Y, and/or at least X and no more than Y. For example, a length of between 1 and 10 shall include a length of at least 1 (including values greater than 10), a length of less than 10 (including values less than 1), and/or values greater than 1 and less than 10.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the term “about” or “approximately” shall refer to +/−10% of a stated value.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g., efficacious therapy) and/or to prevent or otherwise reduce (hereinafter “prevent”) an undesired event (e.g., a device and/or clinical adverse event). In some embodiments, a system parameter is maintained above a first threshold (e.g., above a first temperature threshold to cause a desired therapeutic effect to tissue) and below a second threshold (e.g., below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described herein.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy and/or data. Alternatively or additionally, a functional element (e.g. a functional element comprising a sensor) can be configured to record one or more parameters, such as a patient physiologic parameter; a patient anatomical parameter; a patient environment parameter; and/or a system parameter. In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise one or more functional elements.

The term “transducer” where used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as: light (e.g.

a transducer comprising a light emitting diode or light bulb), sound (e.g. a transducer comprising a piezo crystal configured to deliver ultrasound energy); pressure (e.g. an applied pressure or force); heat energy; cryogenic energy; chemical energy; mechanical energy (e.g. a transducer comprising a motor or a solenoid); magnetic energy; and/or a different electrical signal (e.g. different than the input signal to the transducer). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

As used herein, the term “patient” can refer to any human or other animal, whether healthy, ill, and/or suspected of being ill (e.g. undergoing a diagnostic procedure to identify, gather information related to, and/or to prognose a current or future illness). In some embodiments, the patient comprises a mammal. For example, a mammalian patient can comprise, but is not limited to: human; mouse; rat; rabbit; guinea pig; dog; cat; horse; cow; pig; monkey; chimpanzee; baboon; rhesus monkey; sheep; and/or goat.

As used herein, a “medical procedure” can include a diagnostic procedure and/or a therapeutic procedure.

As used herein, the terms “disorder”, “disease”, and “condition” can be used interchangeably for one or more medical conditions a patient.

As used herein, the terms “smart device” and “mobile device” can be used interchangeably to mean any portable computing device comprising a processor and a display. For example, mobile device and smart devices include, but are not limited to, mobile phones, smart phones, smart watches, tablets, laptops, and/or other associated devices as described herein.

It is appreciated that certain features of the inventive concepts, which are, for clarity, described in the context of separate embodiments, may also be provided in combination in a single embodiment. Conversely, various features of the inventive concepts which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the inventive concepts have been simplified to focus on elements that are relevant for a clear understanding of the inventive concepts, while eliminating, for purposes of clarity, other elements that those of ordinary skill in the art will appreciate may also comprise a portion of the inventive concepts. However, because such elements are well known in the art, and because they do not necessarily facilitate a better understanding of the inventive concepts, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Saliva is a clinically informative, biological fluid (biofluid) that is useful for novel approaches to prognosis, laboratory or clinical diagnosis, and monitoring and management of patients with both oral and systemic diseases. It is easily collected and stored and ideal for early detection of disease as it contains specific soluble biological markers (biomarkers). Saliva may contain one or more biomarkers which make it useful for multiplexed assays developed as point-of-care (POC) devices, rapid tests, or in more standardized formats for centralized clinical laboratory operations. Ultimately, salivary diagnostics may be incorporated as part of disease diagnosis, clinical monitoring and for making important clinical decisions for patient care.

Salivary diagnostics may be considered a subset of the larger field of molecular diagnostics, now recognized as a central player in a wide variety of biomedical basic and clinical areas. Molecular diagnostics feeds into a wide range of disciplines including drug development, personalized medicine (pharmacogenomics) and plays a major role in discovery of biomarkers for the diagnosis of oral and systemic diseases. This is especially true given that biomarkers present in blood and urine can also be detected in a sample of saliva.

Oral tests may be used for the detection of antibodies to the Human Immunodeficiency Virus (HIV) and may be as sensitive and specific as a blood test. This discovery has led to an increase in HIV testing at a variety of locations including emergency rooms, sexually transmitted diseases (STD) clinics, community health centers, bath houses, and most recently in dental settings. The ability to accurately detect antibodies to HIV strongly suggests the potential to detect antibodies to many other pathogens.

Oral samples that are useful for the diagnosis of systemic diseases include saliva, gingival crevicular fluid (GCF), oral swabs, dental plaque, and volatiles. Indeed, published data indicates the successful use of all of these types of oral samples to detect or predict susceptibility to systemic diseases.

The ability to accurately assess biomarkers in samples obtained from the oral cavity may depend on the biochemical nature of the marker, the source and type of sample being taken, and the mechanism by which the marker enters the oral cavity. One widely-used type of oral sample is a swab that collects a deoxyribonucleic acid (DNA) sample. This has been employed for many years in forensic studies and more recently for single nucleotide polymorphisms (SNP) analyses for mutations associated with specific diseases. While a DNA sample can be collected from a wide range of sites on/in the human body, oral sampling has been used most often because of the ease of the sampling procedure, i.e., a buccal brushing that is placed in a stabilizing transport medium and sent off to a laboratory for evaluation.

Another widely-used type of oral sample is for the quantitation of steroid hormone levels. Assays are commercially available for cortisol, estriol, estrogen, testosterone, and consistently provide accurate detection of these hormones. However, salivary levels do not correlate well with serum levels in the case of conjugated steroid hormones. Thus, while dehydroepiandrosterone (DHEA) can be reliably monitored in saliva and the measurements reflect blood levels of the hormone, the sulfated derivative of the steroid, DHEA-S, can be measured in saliva, but the levels are not correlated with serum levels. The reason for this discrepancy appears to be the route of entry of the hormone into the oral cavity. DHEA as a steroid can readily cross the phospholipid membrane of epithelial cells lining the blood vessels, so that elevated serum levels translate as elevated saliva levels by simple diffusion of the hormone. The addition of the charged sulfate group, however, impedes membrane transport and the substance detected in saliva likely represents leakage from the blood rather than diffusion.

In various embodiments, detectable biomarkers in saliva may correspond to chronic obstructive pulmonary disease (COPD) and cystic fibrosis, acute myocardial infarction, oral cancer, and HIV, TB and Malaria. In various embodiments, detectable biomarkers within saliva may correspond to hormones, steroids (e.g., cortisol, androgens, testosterone, estriol, estrogen, progesterone, aldosterone, DHEAS), antibodies (e.g., IgG, IgA, sIgA, IgM), growth factors (e.g., EGF, NGF, VEGF, IGF), cytokines and chemokines (e.g., IL-1 beta, IL-8, IL-6, MCP-1, CX3CL1, GRO-1 alpha, troponin I, TNF alpha), nucleic acids (e.g., human DNA, microbial DNA, mRNA, siRNA, micro RNA, miR-125a and miR-200a), proteins (e.g., 100s-1000s), and drugs (e.g., drugs of abuse such as NIDA 5, ethanol, therapeutic drugs, anticonvulsants, antipyretic/analgesics, anti-neoplastic agents, anti-bacterial agents, bronchodilators, cotinine).

In various embodiments, C-reactive protein (CRP) can be monitored in salivary samples. In various embodiments, salivary immunoglobulins levels are known to increase in association with coronary artery disease. In various embodiments, a group of salivary biomarkers can complement findings of an electrocardiogram (ECG) following an acute myocardial infarction, which include CRP, myoglobin and myeloperoxidase, in combination with an ECG. In various embodiments, salivary biomarkers may be incorporated into POC devices for the rapid assessment of cardiovascular disease (CVD) with potential association with distinct disease stages, demonstrating promising results to identify CVD. In various embodiments, elevated salivary lysozyme levels, a biomarker for oral infection and hyperglycemia, may be associated with hypertension, an early stage of CVD.

In various embodiments, salivary markers may be associated with end stage renal disease. In various embodiments, these markers may include cortisol, nitrite, uric acid, sodium, chloride, pH, amylase and/or lactoferrin. In various embodiments, salivary nitrate and uric acid may be monitored. In various embodiments, salivary phosphate may be used as a clinical biomarker for hyperphosphatemia, which is an important contributor to cardiovascular calcification in chronic renal failure (CRF). In various embodiments, both HD and CRF patients may have significantly higher salivary phosphate levels compared with healthy control subjects. In various embodiments, phosphate levels in saliva may have a positive correlation with serum creatinine and the glomerular filtration rate. Thus, salivary phosphate may provide a better marker than serum phosphate for the initiation of treatment of hyperphosphatemia in CRF and HD.

In various embodiments, salivary biomarkers may be used to detect stress and/or pain. In various embodiments, markers for stress or pain may include salivary amylase, cortisol, substance P, lysozyme and secretory IgA. Pain responses in dental pulp have been specifically associated with neuropeptides including calcitonin gene-related peptide (CGRP), substance P, neurokinin A and neurokinin P. In various embodiments, salivary testosterone levels may be associated with increased aggressive behavior and also with athletic activities. In various embodiments, serotonin may be monitored in saliva.

In various embodiments, biomarkers for malignancies may be detected in saliva. In various embodiments, mutations of the tumor suppressor p53 may be detected for salivary gland adenomas or for breast cancer. In various embodiments, elevated levels of the cancer antigen, CA15-3 and the oncogene c-erB2, in woman with breast cancer as compared to controls may be detected. In various embodiments, the tumor marker C125 may be detected in saliva of subjects with malignant ovarian tumors. In various embodiments, down-regulation of the tumor suppressor DMBT1 may be detected in mammary tumors in humans. In various embodiments, four mRNA biomarkers may be detected to distinguish pancreatic cancer subjects from pancreatitis and control subjects.

Because of the large diabetic population, combined with the current epidemic of Type 2 diabetes, an oral test to monitor blood glucose would be highly desirable. Unfortunately, while it is relatively easy to measure salivary glucose, due to the multiple sources of this material in the oral cavity, salivary glucose levels do not correlate with blood glucose levels. In various embodiments, a unique proteomic signature may be determined in saliva from Type-2 diabetics as compared to control saliva (with 65 proteins showing greater than a 2-fold change). Many of these proteins were associated with metabolic and immune regulatory pathways. In various embodiments, exhaled methyl nitrate may be measured to detect Type 1 diabetic hyperglycemia. There may be a correlation between blood glucose levels and exhaled methyl nitrate due to interaction of superoxide dismutase with nitric oxide as a byproduct of elevated oxidative reactions.

In various embodiments, biomarkers may be used to detect major rheumatoid factor diseases include Lupus Erythematosis, Scleroderma, and Sjogren's syndrome. These autoimmune diseases are characterized by the production of auto-antibodies that attack healthy tissue. Sjogren's syndrome is a disease characterized by dryness of the eyes and mouth and it may occur as a primary or a secondary disease. The clinical symptoms in the primary form are more restricted and are associated with lacrimal and salivary gland dryness. In secondary Sjogren's syndrome, patients undergo one of the autoimmune diseases mentioned above before Sjogren's symptoms develop. In contrast, the primary Sjogren's Syndrome (pSS) occurs by itself and it is the third most common autoimmune disease with a reported prevalence between 0.05 and 4.8%, mostly (90%) occurring in women. For decades, the pSS diagnosis has been based on oral examination, detection of blood biomarkers (autoantibodies to self-antigens (SS-A and SS-B), Rheumatoid factor and antinuclear antibodies, and by obtaining a confirmatory salivary gland biopsy. Patients with pSS have forty times higher risk of developing lymphoma, a fatal lymphocytic cancer. In contrast, patients with secondary Sjogren's syndrome tend to have more health problems because they suffer from a primary condition as well as SS. They are also less likely to have the antibodies associated with the pSS. In various embodiments, a panel of salivary biomarkers may be used to distinguish pSS patients from healthy subjects. In various embodiments, whole saliva (i.e., the combination of saliva in the mouth plus saliva from the individual salivary glands) may contain a series of biomarkers that could detect pSS.

In various embodiments, viruses (e.g., at least 23 known viruses) may be identified in salivary samples by specific antibody reactivity, antigen detection, or nucleic acid via PCR. In various embodiments, these viruses include: Herpes viruses, Hepatitis viruses, HIV, Human Papillomavirus (HPV), Influenza virus, and Poliovirus. Fourteen bacterial pathogens were detected (by antibody, antigen or nucleic acid) including Escherichia coli, Mycobacterium tuberculosis, Helicobacter pylori, Treponema pallidum and a wide range of streptococcal species. In various embodiments, non-viral and non-bacterial infectious agents including Candida albicans, Toxoplama gondii, and Schistosoma mansoni were detectable, typically by antibodies to these infectious agents. These pathogens are responsible for both systemic and oral diseases.

In various embodiments, the physicochemical and biochemical properties of saliva along with its complex composition endows this fluid with multiple functions, including: anti-bacterial, anti-viral and anti-fungal properties; buffering capacity for plaque acids; digestive activity (amylase, protease, nuclease enzymes) needed for food mastication; mineralizing agents for protection and repair of hard tissues; lubricant and viscoelastic properties essential for the maintenance of oral health; and protective and repairing fluid for mucosal surfaces. Saliva is a hypotonic biofluid composed of 99.5% water and 0.5% ions (e.g., potassium, calcium, chloride, sodium and phosphates), and organic micro- and macro-molecules (e.g., amino acids, histatins, cystatins, defensins, statherins, lysozyme, proline-rich proteins, carbonic anhydrases, peroxidases, lactoferrin, mucins, secretory immunoglobulins, and lipids among others).

In various embodiments, salivary-derived molecules may be used as diagnostic biomarkers for oral diseases including oral cancer, and conditions caused by fungi (Candida species), viruses (HPV, Epstein-Barr Virus [EBV], Cytomegalovirus [CMV]) and bacteria (multiple species involved in periodontal diseases and caries). In many instances, pathogen-induced oral diseases have been reported as opportunistic or secondary infections and are referred to as early manifestations of the Acquired Immunodeficiency Syndrome (AIDS) in HIV infected subjects. The frequency of many AIDS-related oral manifestations varies, but increases in the absence of highly active antiretroviral therapy (HAART), and may indicate inadequate HAART treatment, development of drug resistance, or therapeutic failure.

In various embodiments, salivary biomarkers may be used to detect oral squamous cell carcinoma (OSCC). OSCC is the most common malignancy of the oral cavity among oral cancers (e.g., adenocarcinomas, lymphomas, sarcomas, verrucous or mucoepidermoid carcinomas, malignant melanoma, and Kaposi's sarcoma), accounting for more than 90% of clinical cases and ranking among the top ten types of cancers worldwide. However, oral cancers have also been reported with less frequency in the oral mucosa, tongue, pharynx, lips, gums, palate, salivary glands, tonsils and sinuses. In various embodiments, these oral cancer biomarkers include: oncogenes (e.g. C-myc, c-Fos, C-Jun), anti-oncogenes (e.g. p53, p16), cytokines (e.g. TGF-β1, IL-8, and IL-1 β), growth factors (e.g. VEGF, EGF and IGF), extracellular matrix-degrading proteinases (MMP1, MMP2, MMP9), hypoxia markers (HIF-α, CA-9), epithelial-mesenchymal transition markers (e.g. E-cadherin, N-cadherin and B-catenin), epithelial tumor factors (CYFRA 21-1), cytokeratins (CK13, 14 and 16), micro RNA molecules and hypermethylation of cancer-related genes (p16 and DAP-K) [43, 82, 83, 84, 85, 86, 87, 88]. These biomarkers have been defined using molecular, transcriptomic, genomic, proteomic, metabolomic and phenotypic techniques.

In various embodiments, the oral cavity of immunocompetent individuals may contain resident microbiota co-existing under a delicate immunophysiological balance and including an important fungal component known as the oral mycobiome. The latter includes culturable and non-culturable fungi, some of which may be pathogenic, causing common oral diseases such as oropharyngeal candidiasis (OPC), frequently observed in immunocompromised individuals. A recent study characterized the oral mycobiome of twenty healthy individuals showing that Candida species were the most frequently isolated fungi (present in 75% of participants), followed by Cladosporium (65%), Aureobasidium, Saccharomycesles (50% for both), Aspergillus (35%), Fusarium (30%), and Cryptococcus (20%). In various embodiments, there are numerous factors that can disturb the balance of microorganisms in the oral microbiome and mycobiome, predisposing individuals to fungal diseases, including: physiological changes that occur in the geriatric and pediatric populations and during pregnancy; disturbances of soft and hard tissues caused by lesions or poor oral hygiene; prolonged use of antibiotics with a broad antimicrobial spectrum; extended use of steroids that impair the immune system; nutritional deficiencies in micro- or macro-nutrients; endocrinological malfunction associated with diseases such as hypothyroidism; chemotherapy and radiotherapy-induced immunosuppression due to cancer; immunodeficiencies caused by pathogens such as the HIV or congenital defects such as thymic aplasia; Xerostomia; autoimmune diseases (Sjogren's syndrome); use of prosthodontic appliances; and diabetes. In various embodiments, biomarkers may be used to detect salivary IgA or IgG antibodies to Candida.

In various embodiments, oral diseases caused by viruses may be detected via salivary biomarkers. In various embodiments, these oral diseases include papillomaviruses (HPV associated with oral cancer -OSSC- and oral warts) and herpesviruses (EBV causing Hairy Leukoplakia and is also associated with various types of lymphoid and epithelial malignancies; Cytomegalovirus [CMV] causing opportunistic infections after solid organ transplantation, retinitis, gastrointestinal and neurological disorders, and oral ulcerations; Herpes Simplex Viruses 1 and 2 [HSV-1 and HSV-2] and Varicella Zoster Virus [VZV] also causing oral ulcerations of the aphthous type; and Human Herpesvirus 8 [HHV-8] causing oral and systemic Kaposi's sarcoma). In various embodiments, oral fluids have also been successfully used in lab diagnostics to detect HIV antigen and antibodies in different nucleic- and immunoassay formats such as qRT-PCR, ELISA, rapid test, POC and microfluidic diagnostic devices. In various embodiments, HIV neutralizing innate immune factors such as defensins may be detected in saliva using sophisticated experimental methodologies such as liquid chromatography-tandem mass spectrometry that involves limited sample manipulation and that can be easily automated. In various embodiments, detection of HPV in saliva samples has utilized nucleic acid assays such as HPV DNA amplification by PCR and this methodology has also been used to detect different HPV types. In various embodiments, antibodies to HPV may be detected through oral fluids. In various embodiments, saliva specimens may be used for direct genotyping of CMV strains in a new PCR-restriction fragment length polymorphism (RFLP) method, coupled with capillary electrophoresis fragment detection for genotyping [126]. In various embodiments, reliable detection and quantification of nucleic acids for HSV-1, HSV-2 and VZV may be performed using oral fluids.

Caries and periodontitis are the most commonly known polymicrobial-driven diseases of the oral cavity. Periodontal disease is a chronic inflammatory process of the periodontium in response to bacterial plaque deposited on the adjacent teeth. Bacterial infections forming biofilms, destroy the alveolar bone and periodontal ligament, induce gingivitis, cause apical migration of the epithelial attachment resulting in the formation of periodontal pockets, and induce irreversible loss and exfoliation of the teeth. If left untreated, gingivitis may progress into periodontitis, leading to tooth loss and severe lesions of soft and hard tissues. Periodontitis is also linked to systemic illness, such as CVD and diabetes. Caries is also caused by bacterial plaque that in combination with fermentable carbohydrates produces acids (e.g., lactic acid) that lower the pH at the surface of the tooth compromising the enamel, dentin and cementum, and ultimately affect the structural integrity of the tooth. In various embodiments, biomarkers such as MMP-8 and -9 (matrix metalloproteinases) may be elevated in subjects with advanced periodontitis, which was predicted when assessing multiple combinations of salivary biomarkers (e.g., MMP-8 and -9 and osteoprotegerin) along with red-complex anaerobic periodontal pathogens (e.g., Porphyromonas gingivalis or Treponema denticola). In various embodiments, disease severity was also predicted when obtaining elevated salivary MMP-8 and T. denticola biofilm levels. In various embodiments, biomarkers (genetically determined oligosaccharides profiles present on salivary glycoproteins) for caries risk assessment with prognostic value for caries susceptibility may be detected.

Accordingly, there is a need for a salivary sensor that can be seated in a user's mouth to detect the presence of one or more salivary biomarkers in real time.

A Method for Thermoforming an Intraoral Monitor with Seam On Inside/Interior

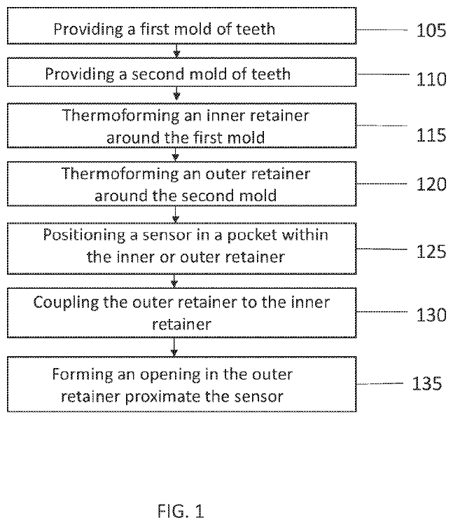

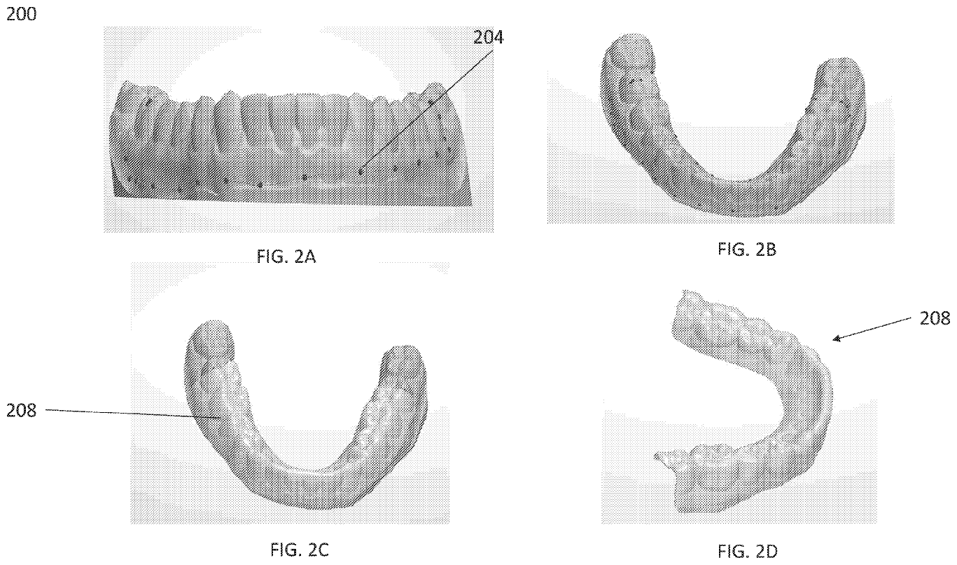

Referring now to FIG. 1, a method 100 for forming an intraoral monitor is shown in graphical representation of a flow chart. Method 100, at step 105 includes, providing a first base model corresponding in shape to at least a portion of a lower set of teeth. The first base model may correspond to one or more computer models generated from 3D scans of at least a portion of a subject's (e.g. human patient) teeth and gums as shown in FIG. 2A-2D. A digital scan 204 of one or more teeth of the patient may be performed. For example, a digital image (e.g. an STL/3D model of teeth) can be generated from an intraoral scanner (e.g. an intraoral scanner may form a portion of the intraoral monitor), such as in FIG. 2A-2B.

In various embodiments, method 100 may include forming an inner retainer model, positioning a sensor model relative to the inner retainer model, checking sensor model placement, cutting the inner retainer model to form the inner retainer base model, and forming the outer retainer base model. The method may further include 3D printing the inner and outer base models, thermoforming plastic over the base models, trimming the plastic from the inner and outer retainers. The method may include placing the cut-away cap in outer retainer pocket, filling the pocket with glue, attaching the inner retainer to the tooth model for mounting, coupling the outer retainer to the inner retainer, slicing a portion of the outer retainer off of the sensor, applying adhesive to the seams formed thereby, and sealing the outer and inner retainers together by laser welding or adhesive. In some embodiments, these steps are performed in the specific temporal sequence listed above. Additionally or alternatively, these steps can be performed in a varying order, with or without time gaps and/or intervening/intermediate steps therebetween.

### Providing a 3D Scan

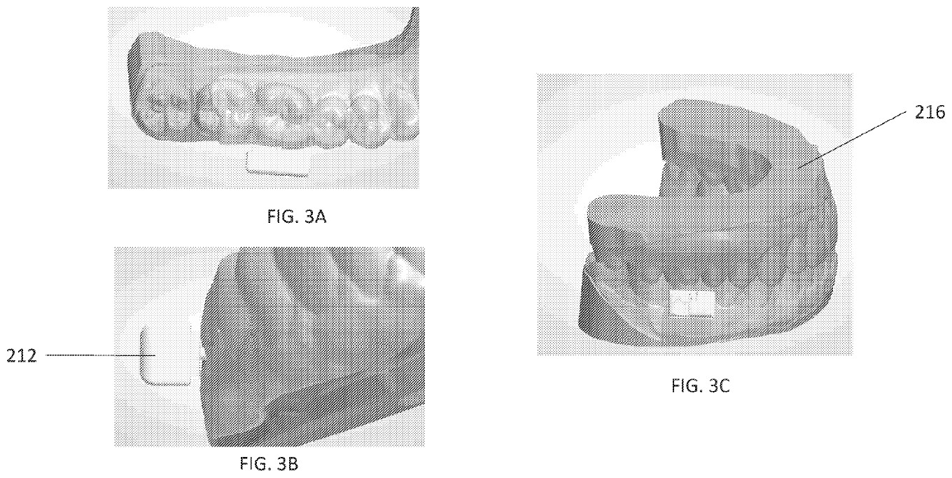

Manufacturing of these devices includes performing a three dimensional in-bite scan or modeling of the user's particular oral anatomy, and can be positioned to avoid interference with the user's bite as shown in FIG. 3C. Additionally, any conductive traces of the circuitry and printed circuit board (PCB) locations are outlined based on this preliminary scan. This step can be performed in isolation, e.g., without the material which forms the structure of the retainer being present. The absence of the retainer material is advantageous in that it maximizes the flexibility of the sensor design such that the placement and orientation of the sensor components can be customized to each user's anatomy to ensure there is no occlusion or contact with the opposing teeth, when the user has a closed bite.

In some embodiments, a CAD file of one or more of the patient's teeth is created, such as via an intraoral 3D scan and/or a 3D scan of a mold (e.g., an alginate mold) of the patient's teeth. The CAD file can be uploaded into memory storage of the patient's clinician (e.g., dentist). A customized intraoral monitor (e.g., or a portion of the device) can be manufactured for that particular patient based on the CAD file, and subsequently shipped to the patient and/or their clinician (e.g., based on address information similarly stored and associated with the CAD file). An alert can be sent to the patient and/or clinician related to delivery, and/or invoicing can automatically be triggered. In some embodiments, replacement devices can be similarly manufactured, delivered, and/or invoiced (e.g., without the need for one or more additional actions by the patient or clinician).

Further in reference to forming the model of a human's teeth, an occlusal model 216 (showing a human's bite) may be performed to test the model of the retainer formed on the teeth model. Forming a digital scan 204 of a person's teeth may include forming a retainer model 208 with one or more thicknesses, the retainer model 208 including the contours of the teeth. Forming the retainer model 208 may include design with a preselection of a retainer material, for example forming the retainer model 208 based on the material selection. Forming the retainer model 208 may include setting a spline to cut within the 3D modeling environment. The spline may correspond to the contours and topology of the teeth and gums proximate the retainer model. For the purposes of this disclosure, retainer, aligner, splint and other terminology for the device that sits on the teeth and holds the intraoral monitor (i.e., sensor) may be used interchangeably without departing from the scope of the disclosure. These structures can have a generally U-shape with two “leg” portions extending longitudinally (e.g. along the molars of a patient) and an arcuate “bridge” portion therebetween (e.g. having a curvature complementary to the patient's radius of curvature from canines-through-incisors). In some embodiments the retainer is a contiguous member that extends along the patient's buccal, occlusal and lingual sides of the teeth. Additionally or alternatively, the retainer can have intermittent gaps/spaces at select locations along its length (on any one, or more, of the buccal, occlusal and lingual sides of the teeth).

### Providing Sensor Model

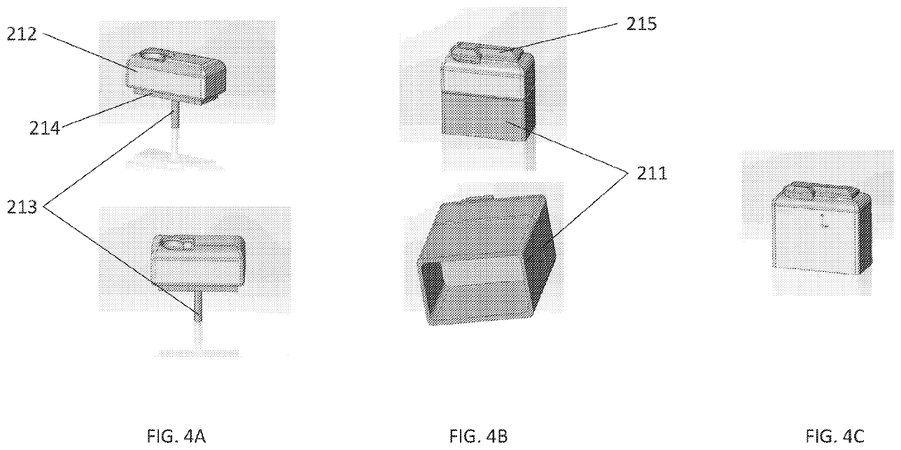

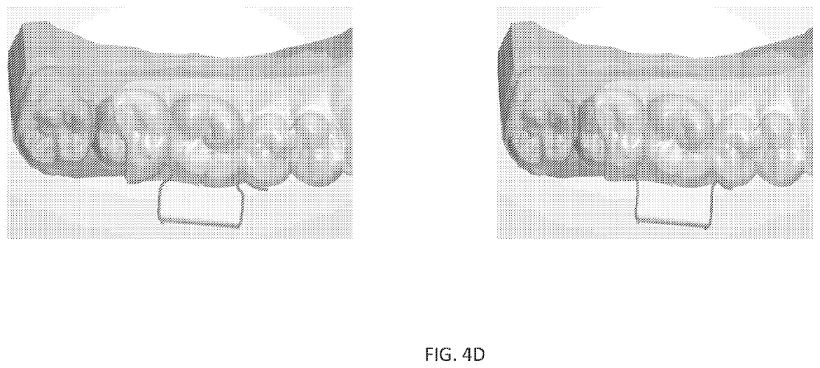

With continued reference to FIGS. 2-3, forming digital scan 204 of the human teeth includes positioning a 3D model of the sensor 212 on the retainer model 208. In various embodiments, the sensor may be any sensor as described herein. In various embodiments, the sensor may be generally box-shape, with a top, bottom, left, right, front and back side with generally rounded edges on at least a subset of the edges formed thereon. The sensor model 212 may include one or more positioning features as shown in FIGS. 3A-4C. In various embodiments, the sensor model 212 may include one or more positioning features such as pegs, posts, boxes, standoffs, cases, or the like. In various embodiments, the sensor model may include one or more features designed to be removed once a tangible product is formed based on the model. Gingival-occlusal position of the sensor can be configured with the top (closest to occlusal plane) surface of the sensor positioned slightly below occlusion from the maxillary arch, leaving slightly more space between occluding arch and top sensor surface than the thickness of the thermoformed plastic, that is to say that the retainer will terminate in the occlusal plane above the top surface of the sensor. In various embodiments, mesial-distal position of the sensor can be centered on the first or second molar, rotated so the sensor is in-line with the neighboring teeth. Gingival occlusal rotation should be so that top of sensor is close to contacting inner retainer on tooth but not quite, and close to contacting inner retainer on gums but not quite. One of ordinary skill in the art would appreciate that the figures and description of the positioning of the sensor are exemplary only, and provide only for a general description of the placement of the sensor. One of ordinary skill in the art would appreciate that an individual's teeth and mouth are unique, and may provide necessity to position the sensor accordingly.

### Positioning the Sensor Model

The sensor model 212 may include one or more positioning features as shown in FIGS. 3A-4C as disclosed above. In various embodiments, the sensor model may include one or more positioning features such as pegs, posts, boxes, standoffs, cases, or the like. In various embodiments, the sensor model may include one or more features designed to be removed once a tangible product is formed based on the retainer model 208. As described herein, the sensor model 212 may include geometry based on the sensor enclosure, a spacing block 214 and a connector rod 213. In various embodiments, connector rod 213 may be placed at the planar center of a bottom side of the sensor model 212. Connector rod 213 may be formed perpendicularly to the planar surface on which it is disposed. Connector rod 213 may be configured to locate said sensor model 212 within the retainer model 208, as shown in FIG. 3A-3B, wherein the connector rod punctures the retainer model 208.

Connector rod 213 may be a feature that can be laterally spaced or placed proximate any tooth in the model, as shown in FIG. 3A. In various embodiments, connector rod 213 can be placed within a single tooth, between adjacent teeth, or through various teeth, according to the scan of the human's teeth and mouth, which are unique to the patient. In various embodiments, the spacing block 214 ensures there is enough space between an inner retainer and sensor enclosure during positioning to account for tolerances. The spacing block 214 provides a feature to space the sensor on the teeth and gums to account for lateral placement of the sensor in the occlusal plane such that when the spacing block 214 contacts one or more contours of the teeth and gums, the sensor is placed appropriately for manufacturing of the physical retainer. In various embodiments the spacing block 214 may have the same thickness as the selected plastic for thermoforming. In various embodiments, spacing block 214 may have a smaller thickness than the thermoforming plastic such as to create a recess. In various embodiments, spacing block 214 may have a larger thickness than the thermoforming plastic, thereby spacing the sensor model 212 with excess room in a pocket. If too thin, sensor will hit inner retainer and there will be a gap between inner and outer retainer instead of flush. Spacing block 214 can be for example 0-1 mm thick. In various embodiments, the spacing block 214 may be 0.8 mm thick

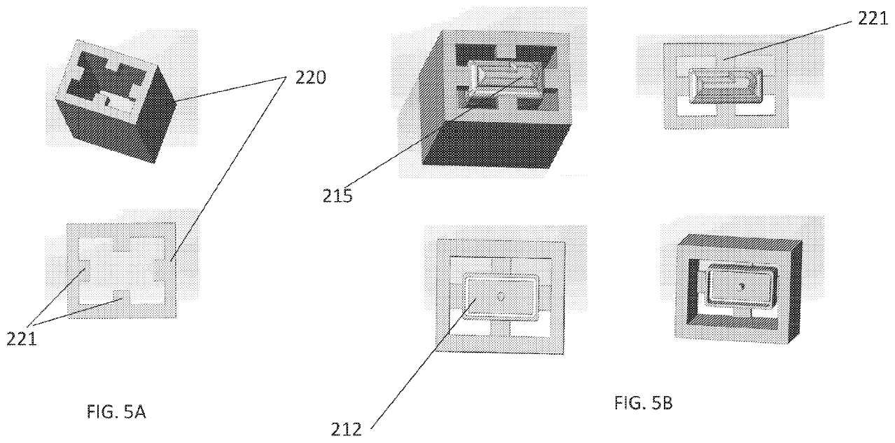

In various embodiments, positioning the sensor model 212 includes geometry for a cut-away cap 215 and an undercut block 211. The cut-away cap 215 may be positioned on sensor model 212 surface with the transducer, in embodiments. The cut-away cap 215 may be cut away from the physical retainer during a downstream step in the manufacturing process to expose transducer and make an opening planar in the outer retainer such that no gaps or jagged edges remain, thereby sealing the area against bacteria/debris can settle within the gaps created by the retainer and the sensor, which will be described below. The undercut block 211 prevents thermoformed plastic from narrowing between sensor and an inner retainer, effectively narrowing the entrance into the pocket for, thereby preventing the sensor being loaded into the pocket as will be described below. The undercut block 211 ensures that the model forming area where the sensor meets the retainer remains at least as wide as the sensor, as the opening of the pocket needs to be at least the height and width as the sensor to be loaded into the pocket. The undercut block 211 may be configured to have the planform dimensions of the sensor model 212, as shown in FIG. 4B. In various embodiments, the walls of undercut block 211 are thin enough so that the undercut model can be snapped off a third base model (as described herein below) when an outer retainer is removed, so the outer retainer plastic does not have to be twisted or pried off the undercut model, which can cause warping, amongst other deformities. In various embodiments, positioning the sensor model 212 may include a cutout model 220 as shown in FIGS. 5A-5B the cutout model 220 including contact studs 221 to position the sensor model 212 thereon. Contact studs 221 may be formed on one or more walls of the cutout model 220, including opposing walls. IN various embodiments, contact studs may be formed on three of the four walls of the cutout model 220. In various embodiments, contact studs 221 may be formed on all four walls of the cutout model 220. In various embodiments, contact studs 221 may be formed a first side of the cutout model 220, the cutout model extending perpendicularly therefrom. In various embodiments, the contact studs 221 may be formed along the cutout model 220, such as within the cutout model 220 at a third depth, the third depth between the first depth and the second depth. Contact studs 221 may each be similarly sized and locate the sensor model 212 within cutout model 220. The cutout model 220 may define a cut in the retainer model 208 to position the sensor therein. The contact studs 221 on the cutout model 220 may be formed a first depth and the cutout model 220 may have a second depth, such that the cut formed in the retainer model 208 is not defined by the contact studs 221. In various embodiments, the sensor model 212 only need be positioned once, the retainer model 208 being adjustable from patient to patient thereafter.

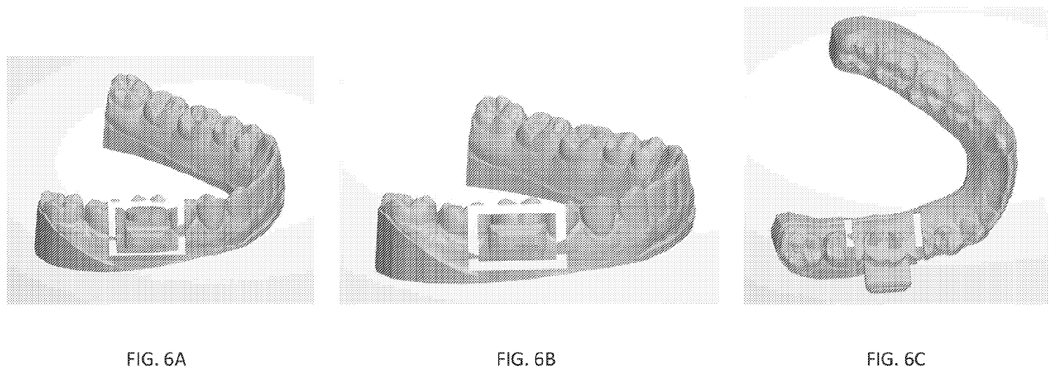

Positioning the sensor model 212 includes coupling the sensor model 212 to the cutout model 220 as shown in FIGS. 5A-5B. The cutout model 220 studs may contact the sensor model 212 as shown. The walls of undercut block may be formed thin enough so that the undercut model can be snapped off the tooth model when the outer retainer is removed. Sensor model 212 may have the sensor enclosure completely aligned in sensor placement model and undercut model. The cutout model can be employed with the studs of FIGS. 5A-5B aligned to the side of the sensor. In various embodiments, the sensor model 212 is a visual model that aids during placement on the retainer model 208. In various embodiments, the undercut model is an additive model that possesses geometry that will be added to the retainer model 208. In various embodiments, the cutout model is a subtractive model which possesses geometry that will be physically removed from the retainer model 208 as shown in FIGS. 6A-6C.

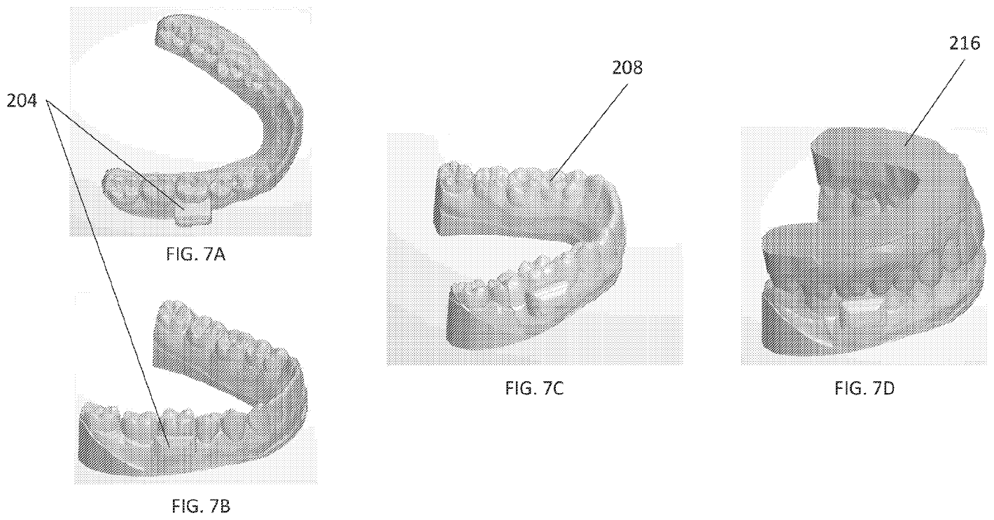

In various embodiments, forming the first base model in FIG. 7A includes forming the retainer model 208 in FIG. 7C and performing a clearance check with an occlusal model 216, as shown in FIG. 7D. The digital scan 204 may be combined with sensor attachment models as described herein. The combination with the original digital scan 204 eliminates the cut caused by the cut model, thereby forming retainer model 208.

Forming a retainer model 208 over the sensor attachment as shown in the previous figures confirms that there is enough space between sensor and occluding arch when outer retainer plastic is formed over sensor. If there is not enough space, the occluding arch will bite into the outer retainer material and the sensor must be placed again, moved down towards the gingiva. In various embodiments, retainer model 208 that is formed in this step can be deleted provided the sensor is placed correctly, wherein the model serves as a visual or analytical interference check.

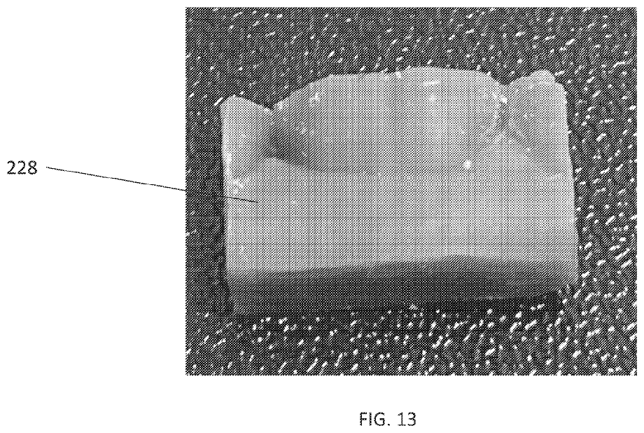

Forming the first base model 228 may include 3D printing or otherwise additively manufacturing first base model 228. Forming first base model 228 may include subtractively manufacturing the first base model 228 such as machining, turning, or otherwise cutting a stock material to final shape of first base model 228. In various embodiments, a 3D printed first base model 228 is shown in FIG. 13.

### Providing the Second Base Model by Manipulation of the First Base Model

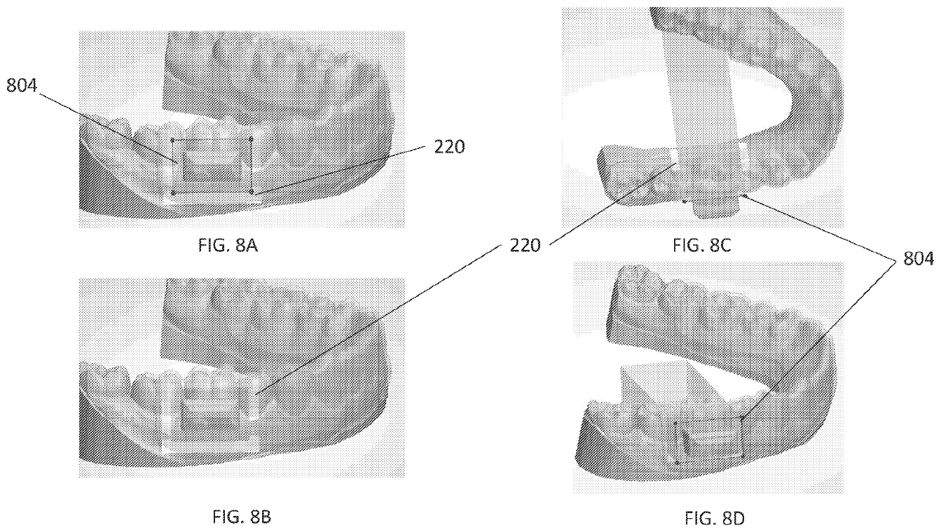

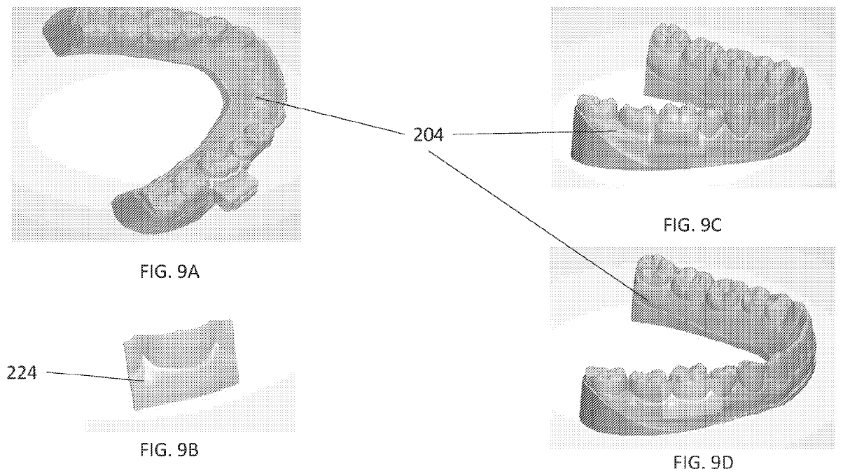

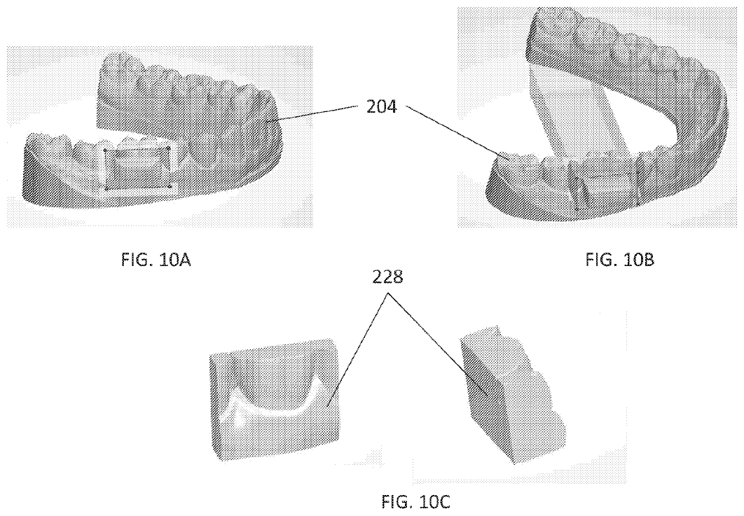

With continued reference to FIG. 1, method 100 includes, at step 110, providing a second base model 232 with at least a portion of contours of a human set of teeth. The second base model 232 may be formed by cutting a portion of inner retainer model 224, and the inner retainer model 224 may be formed from cutting retainer model 208. After the cut, inner retainer model 224 is offset the exact specified distance from each side of the sensor model 212, as specified by the cutout model 220 as previously described. The sensor model 212 may be oriented such that the top face of the sensor model 212 is parallel and the sides are perpendicular to a screen on which a user is working, thereby ensuring a normal cut made by cut points 804 to the retainer model 208. Forming the inner retainer model 224 may include placing four cut points 804 on the four inner corners of the cut caused by the cutout model 220, the process for which is shown in FIGS. 8A-8D. The inner retainer model 224 has at least a portion of the contours of teeth and gums, as shown in FIGS. 9A-9D. The second base model 232 is a contour model of at least a portion of human teeth, the same teeth as first base model 204 with the addition of inner retainer model 224, thus when a plastic casing is formed around second base model 232, the plastic is formed with an offset matching in shape and size to an inner retainer, which will be affixed thereto to close the pocket in which the sensor is disposed and attached. Forming the first base model 232 further may include forming an inner retainer model 224 as shown in FIG. 9B-9D. The inner retainer model 224 may be formed with a flat or generally flat back or at least one generally flat side, shown in FIG. 10C. Forming the second base model 232 may include 3D printing or otherwise additively manufacturing second base model 232. Forming second base model 232 may include subtractively manufacturing the second base model 232 such as machining, turning, or otherwise cutting a stock material to final shape of second base model 232. In various embodiments, a 3D printed second base model 232 is shown in FIGS. 12A and 12B.

### Providing the Retainers

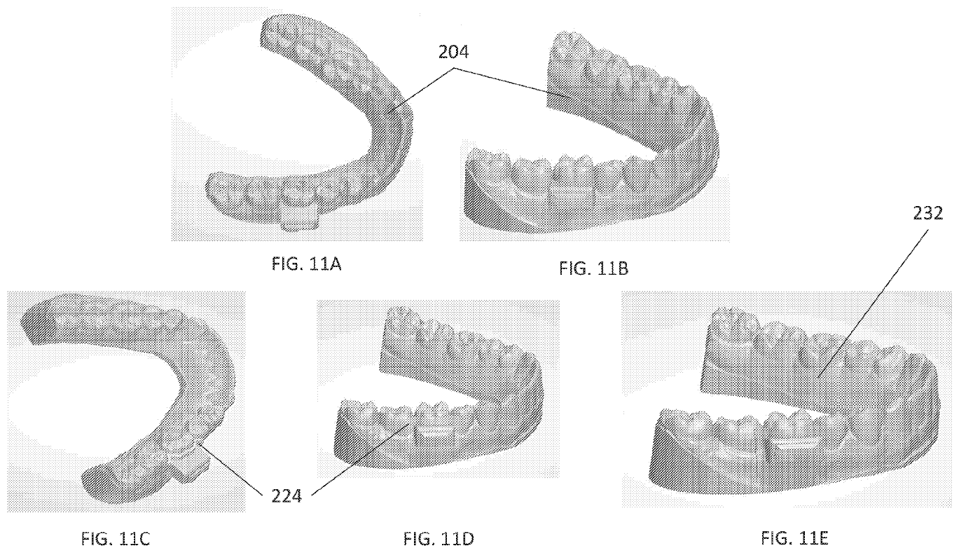

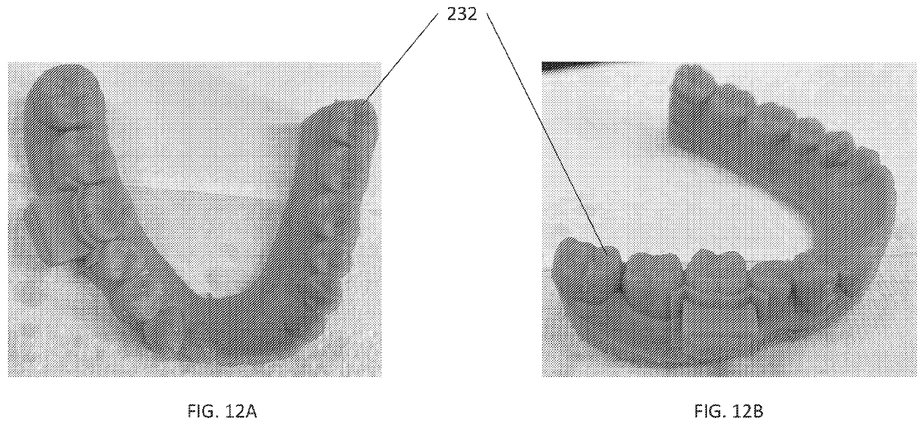

With continued reference to FIG. 1, method 100 includes, at step 115, thermoforming an inner retainer 224 around the first base model 228. Inner retainer 224 may be configured to abut the buccal side of at least a portion of a human's teeth and gums, as shown in the various figures shown herein, namely FIGS. 9A-11E. For the purposes of this disclosure, thermoforming is a manufacturing process wherein a plastic sheet is heated to a pliable forming temperature and formed to a shape over a mold (the first and second base models). The sheet, or “film” when referring to thinner gauges and certain material types, is heated in an oven to a high-enough temperature that permits it to be stretched into or onto a mold and cooled to a finished shape. In various embodiments the plastic may be subjected to a pressure differential or vacuum to absorb shape of the models.

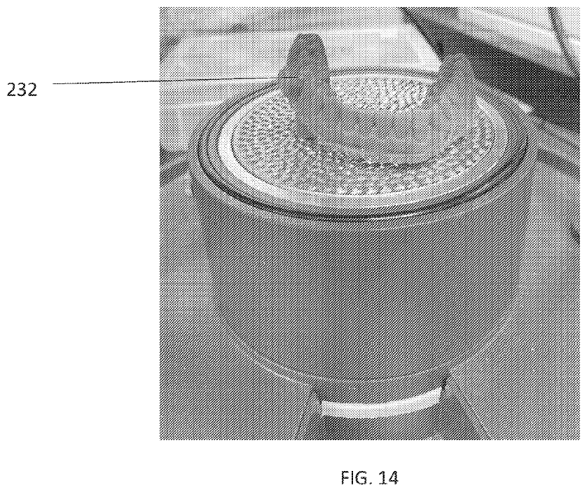

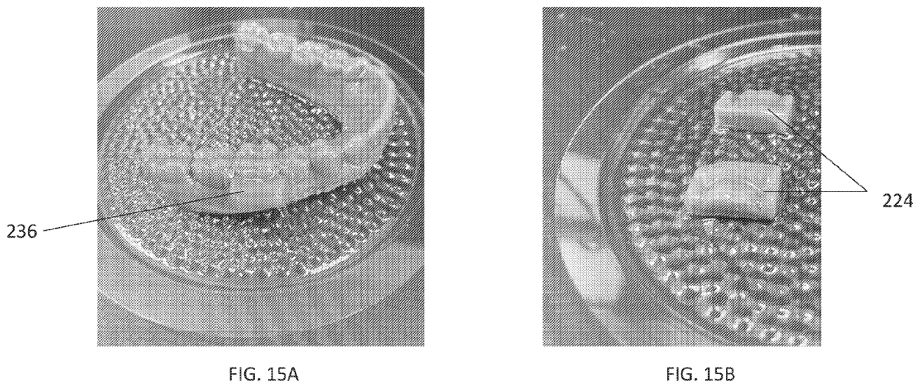

In various embodiments, small tabletop or lab size machines may be used to heat cut sections of plastic sheet and stretch it over a mold using vacuum. In various embodiments, large production machines may be utilized to heat and form the plastic sheet and trim the formed parts from the sheet in a continuous high-speed process and can produce many thousands of finished parts per hour depending on the machine and mold size and the size of the parts being formed. The first base model 228 has the contours of a portion of teeth and gums, having a projected rectangular shape corresponding to the inner retainer. Thermoforming an inner retainer 224 around first base model 228 may be performed using a thermoforming machine. An exemplary embodiment of a model on the thermoforming machine before thermoforming of plastic is initiated is shown in FIG. 14. Thermoforming inner retainer 224 is shown in FIG. 15B, a set of two inner retainers 224 are shown, a benefit of using a thermoforming machine with a plate much larger than the second base model 228.

Although the exemplary embodiment shown includes a polymeric retainer structure, the devices disclosed herein can be fabricated with one or more of many materials such as metal, glass, reinforced fibers, carbon fiber, composites, reinforced composites, aluminum, biological materials, and combinations thereof. The device can be shaped in many ways, such as with thermoforming or direct fabrication (e.g., 3D printing, or additive manufacturing). Additionally or alternatively, the retainers or portions thereof can be fabricated with machining such as an appliance fabricated from a block of material with computer numeric control machining.

The appliance can be designed specifically to accommodate the teeth of the user (e.g., the topography of the tooth receiving cavities matches the topography of the user's teeth), and may be fabricated based on positive or negative models of the user's teeth generated by impression, scanning, models, etc.

Alternatively, the appliance can be a generic device configured to receive the teeth, but not necessarily shaped to match the topography of the user's teeth. In some embodiments, only certain teeth received by the retainer structure will be repositioned while other teeth can provide a base or anchor region for holding the retainer structure in place as it applies force against the tooth or teeth targeted for repositioning.

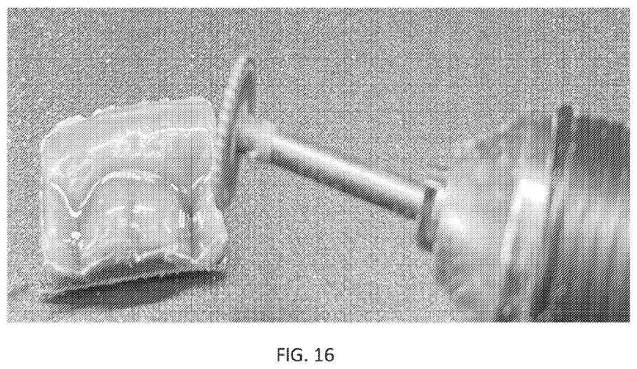

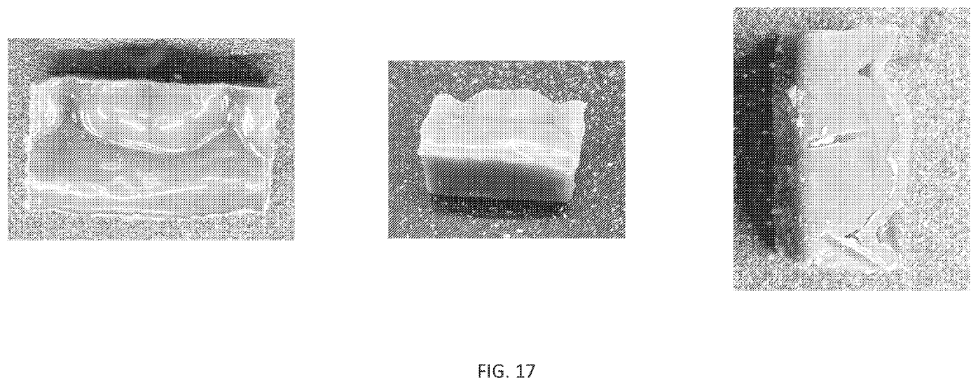

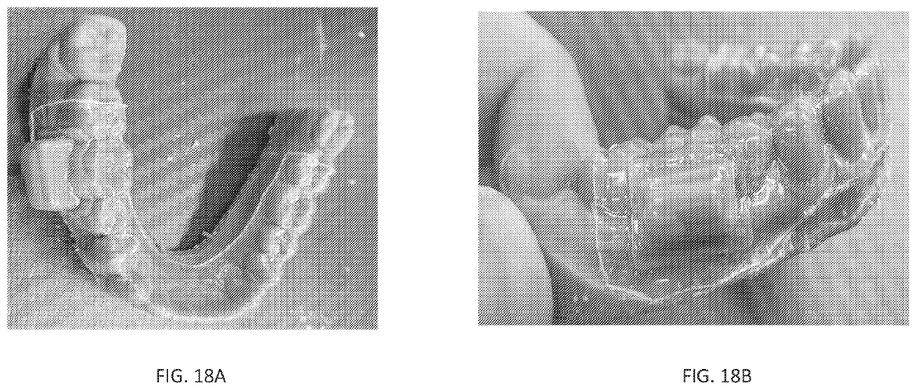

Forming the inner retainer 224 may include trimming a perimeter portion of the inner retainer 224 formed after thermoforming. The plastic vacuum-shaped to the mold may be cut away and form a jagged edge, therefore trimming using hand tools or power tools to smooth and further refine the shape and edge of inner retainer 224 to user's desire and comfort for the person on which the intraoral monitor 200 is installed. All cuts to the plastic may be performed by lasers, milling machines, or any other automated process utilizing splines imported during the design process, such as the spline in reference to retainer model 208 as described above. The plastic may be trimmed on inner retainer 224 so the plastic only remains on the tooth surface area not on the other sides of the first base model. That is to say that the inner retainer includes only one side of the first base model 228, having no need for the sidewalls on which the plastic was also thermoformed. Trimming the inner retainer 224 can be seen in FIGS. 16-17.

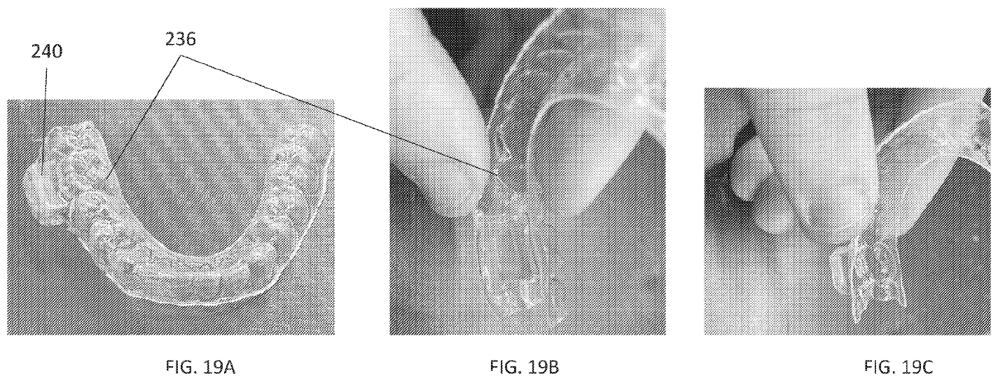

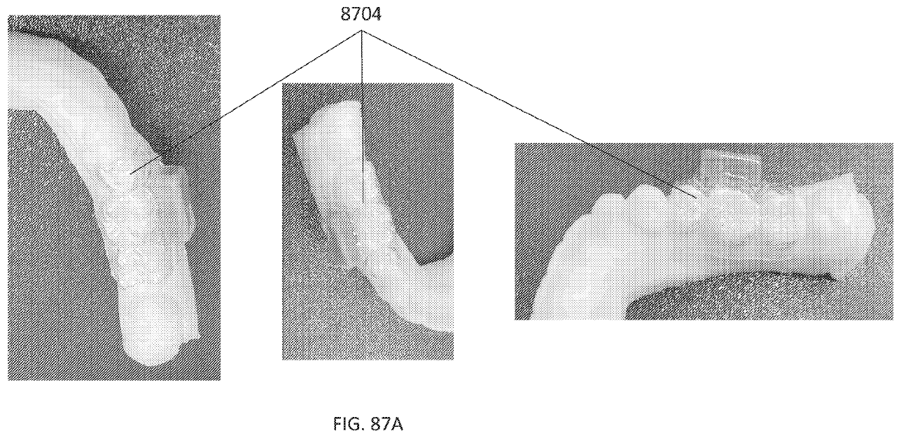

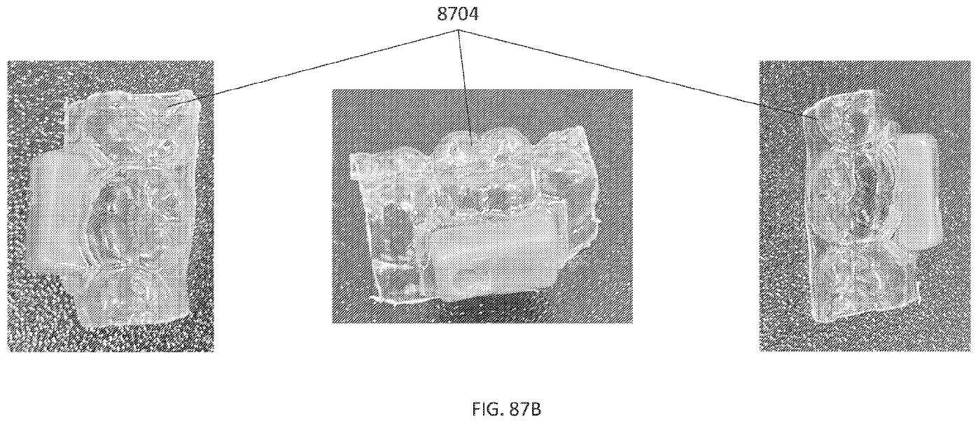

With continued reference to FIG. 1, method 100 includes, at step 120, thermoforming an outer retainer around the second base model as shown in FIGS. 15A and 18A-18B. In various embodiments the plastic may be subjected to a pressure differential or vacuum to impart the shape of the models. Thermoforming the outer retainer 236 includes removing outer retainer 236 from second base model 232 in FIGS. 19A-19C. In various embodiments, the outer retainer 236 is configured to mount the entire set of lower teeth of a human. In various embodiments, the outer retainer 236 may include every contour of second base model 232. In various embodiments, the outer retainer 236 may be configured to mount a subset of teeth, such as 1-3 consecutively teeth as in FIG. 87A-87B.

In various embodiments, small tabletop or lab size machines may be used to heat cut sections of plastic sheet and stretch it over a mold using vacuum. In various embodiments, large production machines may be utilized to heat and form the plastic sheet and trim the formed parts from the sheet in a continuous high-speed process and can produce many thousands of finished parts per hour depending on the machine and mold size and the size of the parts being formed. The first base model 228 has the contours of a portion of teeth and gums, having a projected rectangular shape corresponding to the inner retainer. Thermoforming an inner retainer 224 around first base model 228 may be performed using a thermoforming machine. An exemplary embodiment of a model on the thermoforming machine before thermoforming of plastic is initiated is shown in FIG. 14. Thermoforming inner retainer 224 is shown in FIG. 15B, a set of two inner retainers 224 are shown, a benefit of using a thermoforming machine with a plate much larger than the second base model 228.

Although the exemplary embodiment shown includes a polymeric retainer structure, the devices disclosed herein can be fabricated with one or more of many materials such as metal, glass, reinforced fibers, carbon fiber, composites, reinforced composites, aluminum, biological materials, and combinations thereof. The device can be shaped in many ways, such as with thermoforming or direct fabrication (e.g., 3D printing, or additive manufacturing). Additionally or alternatively, the retainers or portions thereof can be fabricated with machining such as an appliance fabricated from a block of material with computer numeric control machining.

The appliance can be designed specifically to accommodate the teeth of the user (e.g., the topography of the tooth receiving cavities matches the topography of the user's teeth), and may be fabricated based on positive or negative models of the user's teeth generated by impression, scanning, models, etc.

Alternatively, the appliance can be a generic device configured to receive the teeth, but not necessarily shaped to match the topography of the user's teeth. In some embodiments, only certain teeth received by the retainer structure will be repositioned while other teeth can provide a base or anchor region for holding the retainer structure in place as it applies force against the tooth or teeth targeted for repositioning.

Forming outer retainer 236 may include forming outer retainer 236 having the contours of at least a portion of a lower set of teeth. Forming outer retainer 236 may include forming outer retainer 236 having the contours of at least a portion of an upper set of teeth. Forming outer retainer 236 may include forming outer retainer 236 having the contours of at least a portion of a full set of lower teeth and gums. Forming outer retainer 236 may include forming outer retainer 236 having the contours of at least a portion of a full set of upper teeth and gums. Forming outer retainer 236 having an outer surface having a buccal side, an occlusal side and a lingual side. Forming outer retainer 236 having an inner surface abutting and corresponding to in shape and contour of the teeth. Forming outer retainer 236 includes forming a pocket 240 disposed on the buccal side of the outer retainer 236. The pocket 240 may form the complimentary shape of the sensor. Pocket 240 protrudes through the outer surface of outer retainer 236 on the buccal side, thereby forming a negative space on the inner surface of outer retainer 236. Pocket 240 is contiguous with the entirety of the outer retainer and formed from the same material and in the same step as forming outer retainer 236.

### Placing Sensor Inside the Pocket of the Retainer

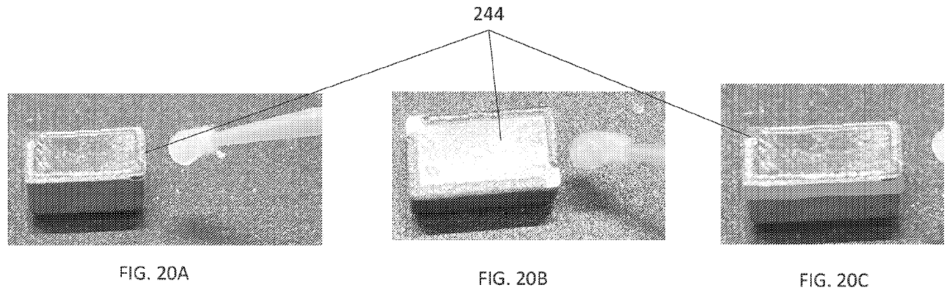

With continued reference to FIG. 1, method 100 includes, at step 125, positioning a sensor 244 in the pocket 240 formed in the outer retainer 236. Note the sensor 244 is now a physical sensor as opposed the sensor model 212. Positioning the sensor 244 in the pocket 240 includes applying an adhesive to the sensor 244 prior to inserting into the pocket 240. Applying an adhesive to sensor 244 may be performed by a cotton swap manually as in FIGS. 20A-20B. Applying an adhesive to sensor 244 may be applied on the edges of the sensor 244 as shown in FIG. 20C. Applying an adhesive to sensor 244 may include applying an adhesive to all sides of the sensor 244. In various embodiments, an adhesive may not be applied to the side of the sensor where the transducer is disposed, thus not introducing an adhesive to the sensitive portion of the device. In various embodiments, applying an adhesive to the sensor 244 includes applying UV an adhesive on walls of sensor to ⅓ or ½ way down the wall (shown in FIG. 20C). An adhesive is needed so that sensor box walls stick inside the retainer and form a barrier next to the sensor walls. The adhesive may be applied part way down so the adhesive does not run onto the transducer face of sensor 244, interfering with the cut-away post cap step or contaminating the transducer.

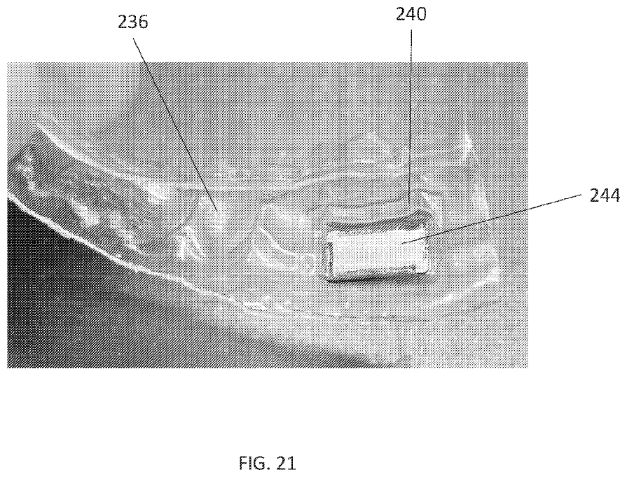

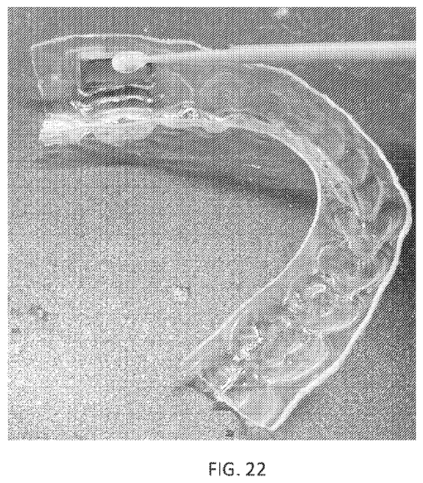

Positioning the sensor 244 into the pocket 240 includes positioning the sensor 244 into pocket 240 transducer side first, thereby exposing an inner surface of the sensor 244 coincident with the inner surface of the outer retainer 236 as shown in FIG. 21-22.

### Coupling the Retainers by Using an Alignment Model

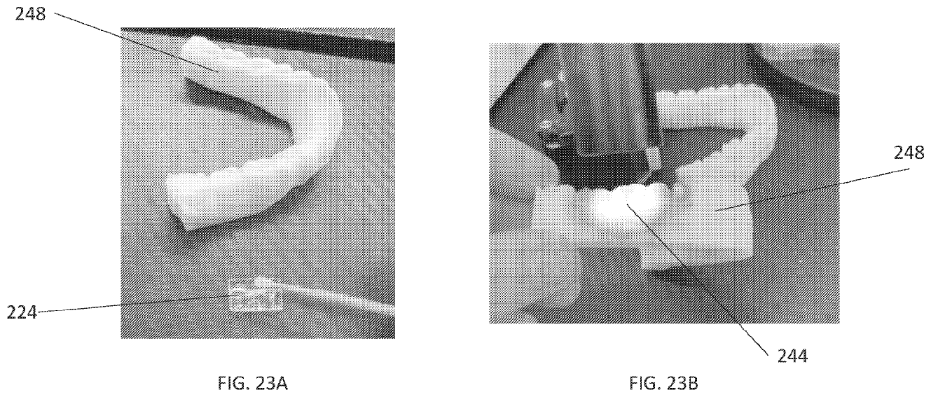

With continued reference to FIG. 1, method 100 includes, at step 130, coupling outer retainer 236 to inner retainer 224. Coupling the inner retainer 224 to outer retainer 236 may include coupling inner retainer 224 to a third base model 248. Third base model 248 may be formed from 3D scan 204, thereby having the exact same contours as inner retainer 224 and outer retainer 236. Third base model 248 may be 3D printed or machined from stock as described in reference to first and second base models 228, 232. Inner retainer 224 may be attached to third base model 248 by UV setting an adhesive, as shown in FIGS. 23A-23B. In various embodiments, during fabrication, a curable polymer (e.g., UV-curable epoxy) may be applied to any of the components described herein to thereby secure that component within its respective housing. For example, inner retainer, outer retainer and any model may be coated with UV-curable epoxy. In various embodiments, the curable polymer may be used to fix a flexible component (e.g., the inner retainer, in embodiments) in a fixed position or shape. In various embodiments, laser welding may be used to connect components, seal components, or otherwise join plastic (and in some embodiments, metallic) components during fabrication. In various embodiments, any gaps may be filled with a potting agent and cured, for example the gap formed in the pocket or the like.

Coupling the inner retainer 224 to third base model 248 includes affixing said inner retainer 224 to a complementary portion of the third base model 248. In various embodiments, inner retainer 224 may be placed on third base model 248 and laid flush thereon, such that there are no gaps formed between the inner retainer 224 and third base model 248, shown in FIG. 23B.

Coupling the inner retainer 224 to the outer retainer 236 may include applying an adhesive to each of the outer retainer 236 and the inner retainer 224. Application of an adhesive to the outer retainer 236 is shown in FIG. 22.

### Closing the Pocket by Coupling the Retainers

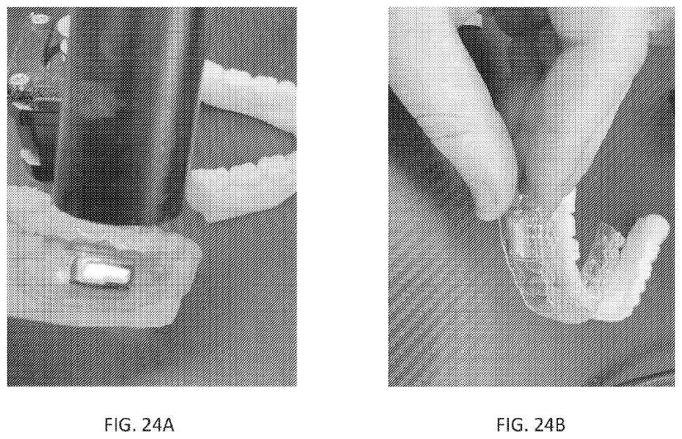

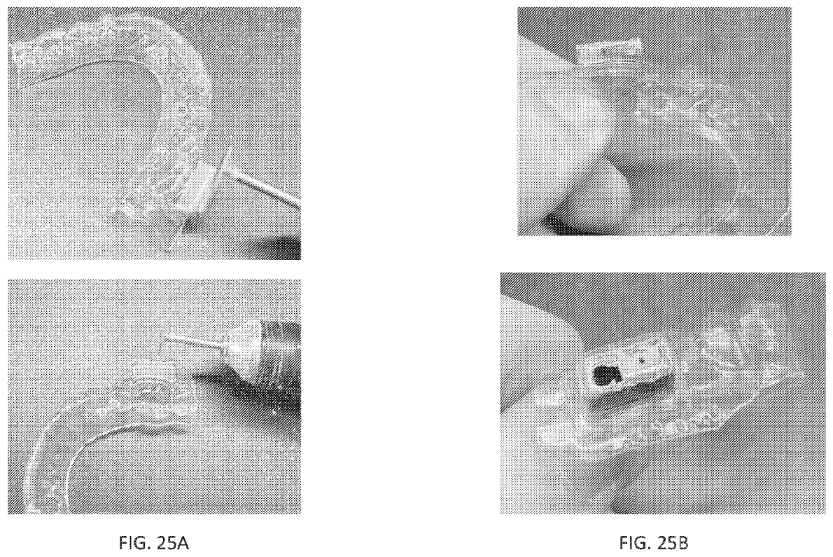

By positioning outer retainer 236 over the inner retainer 224 and third base model 248, thereby closing the pocket 240 with inner retainer 224. In various embodiments, aligning outer retainer 236 over inner retainer 224 includes aligning inner retainer 224 with pocket 240, thereby closing the pocket 240 with inner retainer 224 as a cover to enclose sensor 244 within the pocket 240. Aligning the outer retainer 236 over the attached inner retainer 224 and exposing the adhesive therebetween to ultraviolet (UV) light, thereby curing the adhesive as in FIGS. 24A and 24B. In various embodiments, outer retainer 236 and inner retainer 224 may be welded via one or more lasers. In various embodiments, the laser may be automated or manually directed to the area to be welded. In various embodiments, a portion of each of the outer retainer 236 and inner retainer 224 may overlap, the overlapping sections disposed about the pocket in outer retainer 236. In various embodiments, the overlapping portions of the outer retainer 236 and inner retainer 224 may be exposed to a laser beam, thereby welding the outer retainer 236 and inner retainer 224 together.

### Trimming the Retainer Covering the Sensor

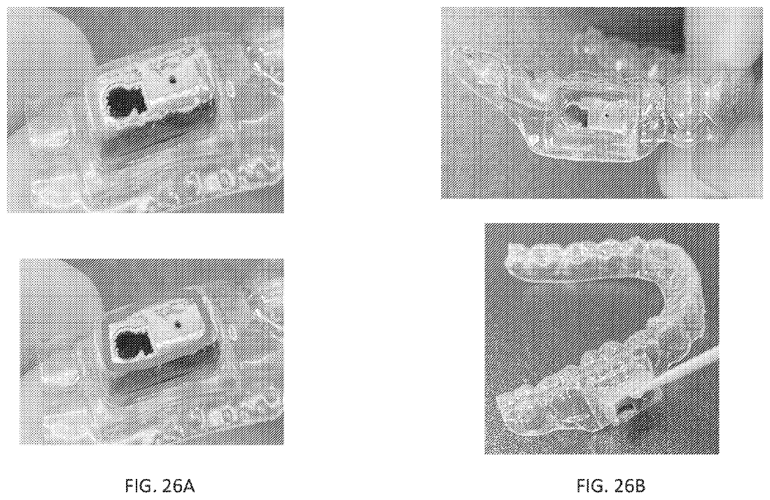

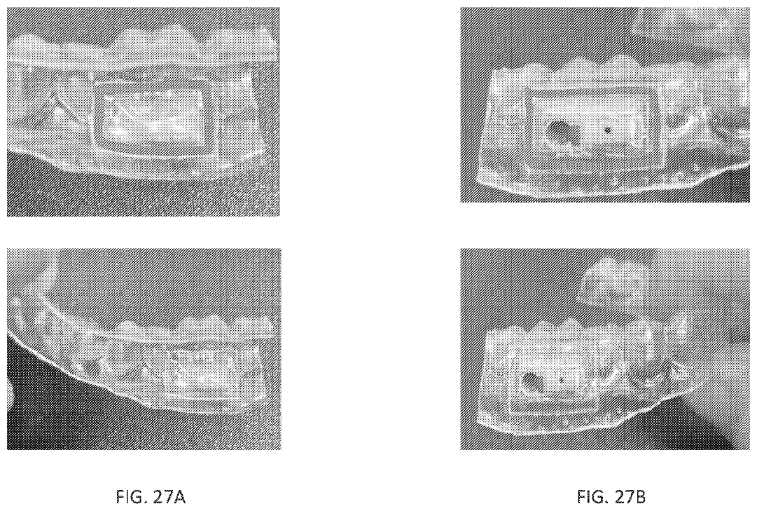

With continued reference to FIG. 1, method 100, includes at step 135, removing a portion of outer retainer 236 proximate the sensor 244 to form an opening in the outer retainer 236. In various embodiments, removing a portion of the outer retainer 236 may include destructively removing a portion of the plastic via one or more saw, sawzalls, cutting wheels, grinders, knives, blades or another tool to cut the outer retainer 236. In various embodiments, removing the portion of the outer retainer 236 may include utilizing hand reciprocating, oscillating, rotating, or vibrating power tools to remove the portion of plastic as shown in FIG. 25A. Removing the portion of outer retainer 236 may include exposing a portion of the sensor 244 proximate the transducer as described herein, thereby exposing the transducer to the inside of the mouth of the human on which it is installed. The plastic of the outer retainer 236 is therefore removed from the transducer side of the sensor 244 as shown in FIG. 25B. There may be a seam formed between the cut edge of the portion of the outer retainer 236 and sensor 244. An adhesive may be applied to the seam as shown in FIGS. 26A and 26B. Applying an adhesive to the seam may include sealing the seam against the ingress of one or more contaminants, fluids, portions of food or the like. Sealing the seam may include funneling saliva to the transducer of the sensor 244. In various embodiments, applying an adhesive to the seam may include applying an adhesive to the seam manually such as utilizing a toothpick or other implement with a relatively small tip to apply an adhesive to the seam area in FIG. 26B. In various embodiments, applying an adhesive to the seam may include applying the adhesive automatically through one or more automated processes or robotically.

Method for Thermoforming an Intraoral Monitor with Seam On Outside/Exterior

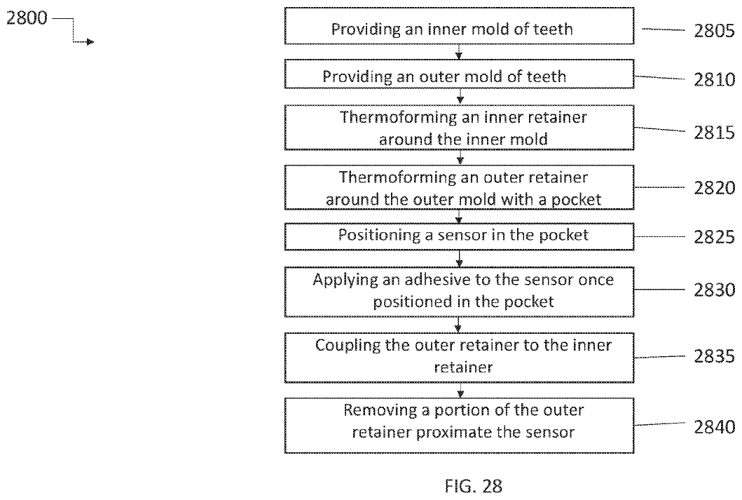

Referring now to FIG. 28, a method 2800 for forming an intraoral monitor is presented in flow chart form. Method 2800 includes at step 2805, providing an inner thermoform mold corresponding in shape to at least a portion of a lower set of teeth. Inner thermoform mold may be formed from a scan 2804.

In various embodiments, method 2800 may include forming an inner retainer model, positioning a sensor model relative to the inner retainer model and attaching thereto, combining the scan model with the inner retainer model, checking sensor model placement, and cutting the inner retainer model to form the outer thermoform model. The method may further include 3D printing the inner and outer thermoform molds, thermoforming plastic over the molds, trimming the plastic from the inner and outer retainers. The method may include placing the cut-away cap in outer retainer pocket, filling the pocket with glue, coupling the outer retainer to the inner retainer, slicing a portion of the outer retainer off of the sensor, applying adhesive to the seams formed thereby, and sealing the outer and inner retainers together by laser welding or adhesive. In some embodiments, these steps are performed in the specific temporal sequence listed above. Additionally or alternatively, these steps can be performed in a varying order, with or without time gaps and/or intervening/intermediate steps therebetween.

### Providing an Inner Thermoform Mold

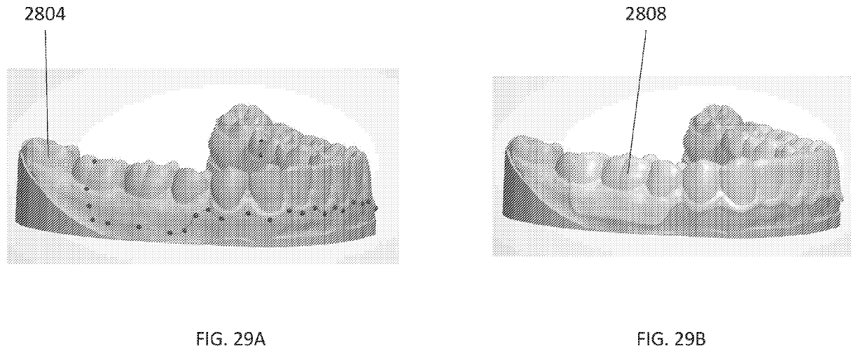

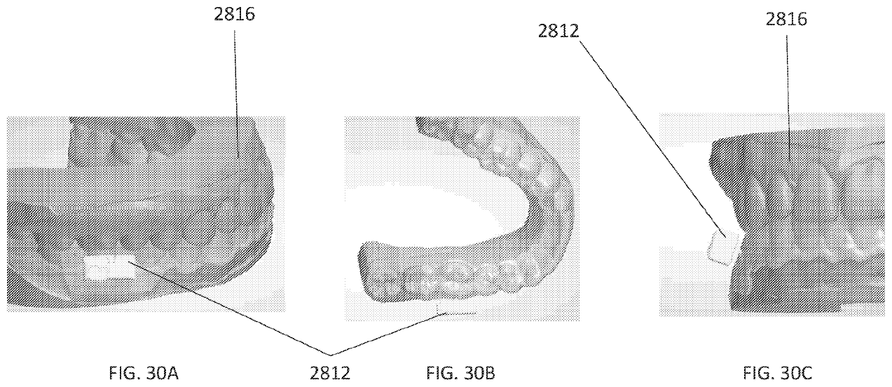

The inner thermoform mold may be based on one or more computer models generated from 3D scans of at least a portion of a human's teeth and gums as shown in FIG. 29A. A digital scan 2804 of one or more teeth of the patient may be performed in or out of a dental office. For example, a digital image (e.g. an STL/3D model of teeth) can be generated from an intraoral scanner (e.g. an intraoral scanner may form a portion of the intraoral monitor), such as in FIG. 29A-30C.

Scan 2804 may be a three dimensional in-bite scan or modeling of the user's particular oral anatomy, and can be positioned to avoid interference with the user's bite as shown in FIG. 30A-30C. Additionally, any conductive traces of the circuitry and printed circuit board (PCB) locations are outlined based on this preliminary scan. This step can be performed in isolation, e.g., without the material which forms the structure of the retainer being present. The absence of the retainer material is advantageous in that it maximizes the flexibility of the sensor design such that the placement and orientation of the sensor components can be customized to each user's anatomy to ensure there is no occlusion or contact with the opposing teeth, when the user has a closed bite.

In some embodiments, a CAD file of one or more of the patient's teeth is created, such as via an intraoral 3D scan and/or a 3D scan of a mold (e.g., an alginate mold) of the patient's teeth. The CAD file can be uploaded into memory storage of the patient's clinician (e.g., dentist). A customized intraoral monitor (e.g., or a portion of the device) can be manufactured for that particular patient based on the CAD file, and subsequently shipped to the patient and/or their clinician (e.g., based on address information similarly stored and associated with the CAD file). An alert can be sent to the patient and/or clinician related to delivery, and/or invoicing can automatically be triggered. In some embodiments, replacement devices can be similarly manufactured, delivered, and/or invoiced (e.g., without the need for one or more additional actions by the patient or clinician).

Further in reference to forming the model of a human's teeth, an occlusal model 2816 (showing a human's bite) may be performed to test the model of the retainer formed on the teeth model. Forming an inner thermoform mold may include forming an inner thermoform model 2808 with one or more thicknesses, the inner thermoform model 2808 including the contours of the teeth and formed over the scan 2804. Forming inner thermoform model 2808 may include design with a preselection of a retainer material, for example forming inner thermoform model 2808 based on the material selection. Forming inner thermoform model 2808 may include setting a spline (like FIG. 29A) to cut within the 3D modeling environment. The spline may correspond to the contours and topology of the teeth and gums proximate the retainer model. For the purposes of this disclosure, retainer, aligner, splint and other terminology for the device that sits on the teeth and holds the intraoral monitor (i.e., sensor) may be used interchangeably without departing from the scope of the disclosure.

### Positioning the Sensor Model

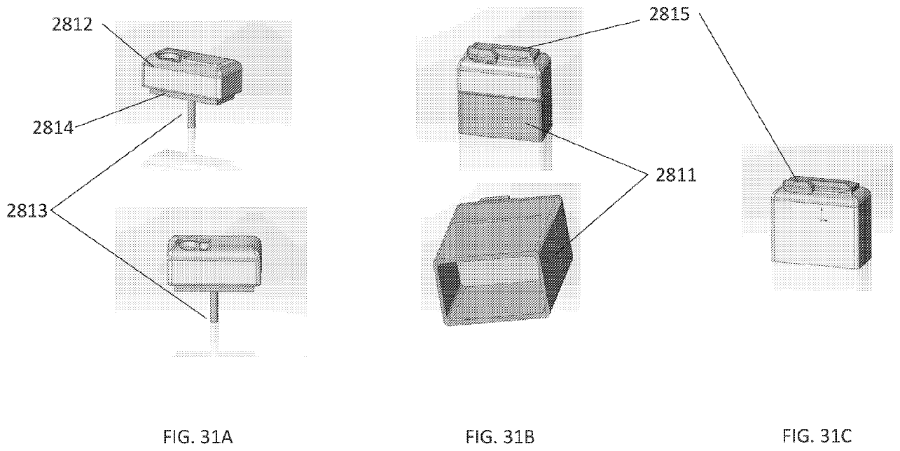

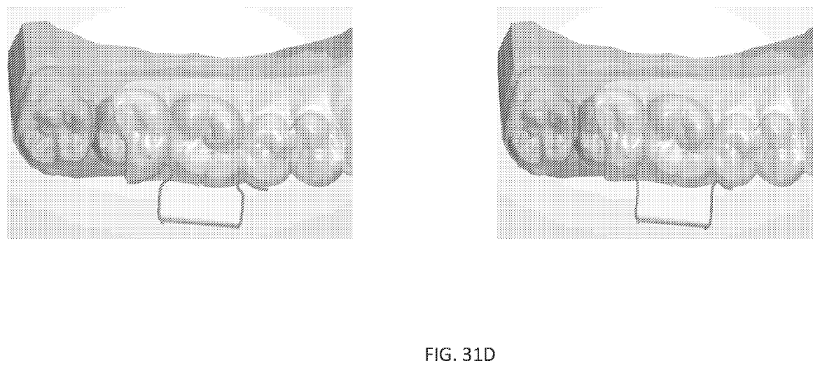

With continued reference to FIG. 28, forming an inner thermoform model 2808 includes positioning a 3D sensor model 2812 on inner thermoform model 2808. In various embodiments, the sensor model corresponds to a physical sensor, said sensor may be any sensor as described herein. In various embodiments, the sensor may be generally box-shape, with a top, bottom, left, right, front and back side with generally rounded edges on at least a subset of the edges formed thereon. The sensor model 2812 may include one or more positioning features as shown in FIGS. 31A-31C. In various embodiments, the sensor model 2812 may include one or more positioning features such as pegs, posts, boxes, standoffs, cases, or the like. As described herein, the sensor model 2812 may include geometry based on the sensor enclosure, a spacing block 2814 and a connector rod 2813. In various embodiments, connector rod 2813 may be placed at the planar center of a bottom side of the sensor model 2812. Connector rod 2813 may be formed perpendicularly to the planar surface on which it is disposed. Connector rod 2813 may be configured to locate said sensor model 2812 within the retainer model 208, as shown in FIG. 3A-3B, wherein the connector rod punctures the retainer model 208. Connector rod 2813 may be a feature that can be laterally spaced or placed proximate any tooth in the model, as shown in FIG. 3A. In various embodiments, connector rod 2813 can be placed within a single tooth, between adjacent teeth, or through various teeth, according to the scan of the human's teeth and mouth, which are unique to the patient. In various embodiments, the spacing block 2814 ensures there is enough space between an inner retainer and sensor enclosure during positioning to account for tolerances. The spacing block 2814 provides a feature to space the sensor on the teeth and gums to account for lateral placement of the sensor in the occlusal plane such that when the spacing block 2814 contacts one or more contours of the teeth and gums, the sensor is placed appropriately for manufacturing of the physical retainer. In various embodiments the spacing block 2814 may have the same thickness as the selected plastic for thermoforming. In various embodiments, spacing block 2814 may have a smaller thickness than the thermoforming plastic such as to create a recess. In various embodiments, spacing block 2814 may have a larger thickness than the thermoforming plastic, thereby spacing the sensor model 2812 with excess room in a pocket. If too thin, sensor will hit inner retainer and there will be a gap between inner and outer retainer instead of flush. Spacing block 2814 can be for example 0-1 mm thick. In various embodiments, the spacing block 2814 may be 0.8 mm thick

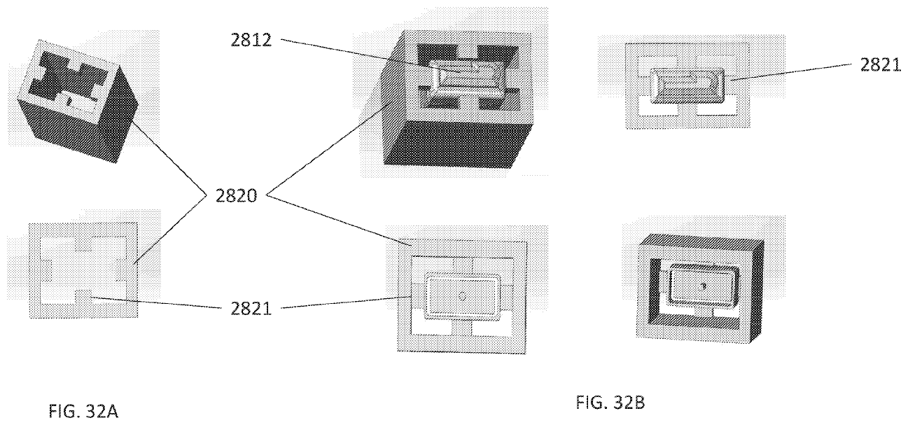

In various embodiments, positioning the sensor model 2812 includes geometry for a cut-away cap 2815 and an undercut block 2811. The cut-away cap 2815 may be positioned on sensor model 2812 surface with the transducer, in embodiments. The cut-away cap 2815 may be cut away from the physical retainer during a downstream step in the manufacturing process to expose transducer and make an opening planar in the outer retainer such that no gaps or jagged edges remain, thereby sealing the area against bacteria/debris can settle within the gaps created by the retainer and the sensor, which will be described below. The undercut block 2811 prevents thermoformed plastic from narrowing between sensor and an inner retainer, effectively narrowing the entrance into the pocket for, thereby preventing the sensor being loaded into the pocket as will be described below. The undercut block 2811 ensures that the model forming area where the sensor meets the retainer remains at least as wide as the sensor, as the opening of the pocket needs to be at least the height and width as the sensor to be loaded into the pocket. The undercut block 2811 may be configured to have the planform dimensions of the sensor model 2812, as shown in FIG. 31B. In various embodiments, the walls of undercut block 2811 are thin enough so that the undercut model can be snapped off a third base model (as described herein below) when an outer retainer is removed, so the outer retainer plastic does not have to be twisted or pried off the undercut model, which can cause warping, amongst other deformities. In various embodiments, positioning the sensor model 2812 may include a cutout model 2820 as shown in FIGS. 32A-32B the cutout model 2820 including contact studs 2821 to position the sensor model 2812 thereon. Contact studs 2821 may be formed on one or more walls of the cutout model 2820, including opposing walls. IN various embodiments, contact studs may be formed on three of the four walls of the cutout model 2820. In various embodiments, contact studs 2821 may be formed on all four walls of the cutout model 2820. In various embodiments, contact studs 2821 may be formed a first side of the cutout model 2820, the cutout model extending perpendicularly therefrom. In various embodiments, the contact studs 2821 may be formed along the cutout model 2820, such as within the cutout model 2820 at a third depth, the third depth between the first depth and the second depth. Contact studs 2821 may each be similarly sized and locate the sensor model 2812 within cutout model 2820. The cutout model 2820 may define a cut in the retainer model 208 to position the sensor therein. The contact studs 2821 on the cutout model 2820 may be formed a first depth and the cutout model 2820 may have a second depth, such that the cut formed in the retainer model 208 is not defined by the contact studs 2821. In various embodiments, the sensor model 2812 only need be positioned once, the retainer model 208 being adjustable from patient to patient thereafter.

In various embodiments, the sensor model may include one or more features designed to be removed once a tangible product is formed based on the model. Gingival-occlusal position of the sensor can be configured so that the top (closest to occlusal plane) surface of the sensor is located slightly below occlusion from the maxillary arch, leaving slightly more space between occluding arch and top sensor surface than the thickness of the thermoformed plastic, that is to say that the retainer will terminate in the occlusal plane above the top surface of the sensor. In various embodiments, mesial-distal position of the sensor is centered on the first or second molar, and rotated so the sensor is in-line with the neighboring teeth. Gingival occlusal rotation can be configured with the top of sensor proximate, but not abutting/contacting, the inner retainer on tooth but not quite, and likewise proximate the inner retainer on gums but spaced so as to not abut/contact directly. One of ordinary skill in the art would appreciate that the figures and description of the positioning of the sensor are exemplary only, and provide only for a general description of the placement of the sensor. One of ordinary skill in the art would appreciate that an individual's teeth and mouth are unique, and may provide necessity to position the sensor accordingly.

### Forming a Model Having Contours of Teeth and Sensors

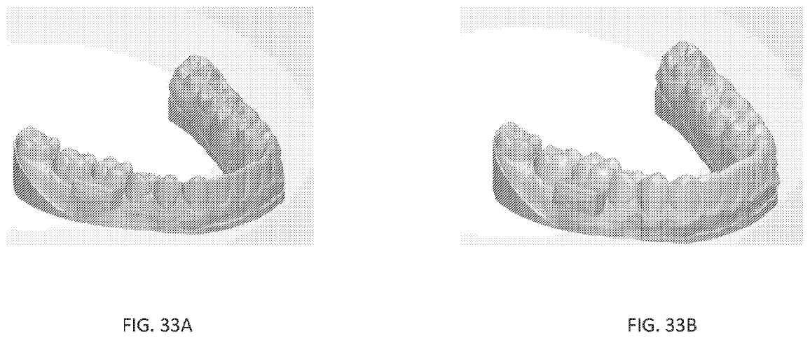

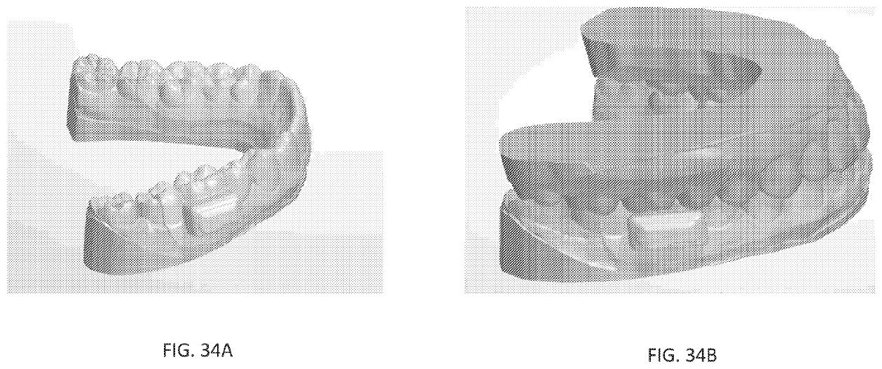

In various embodiments, forming inner thermoform model 2808 includes digitally adding sensor model 2812 to the scan 2804. The resultant inner thermoform model 2808 includes the contours of the lower set of teeth and gums with the placed sensor model 2812 attached thereto. One or more users may then combine this new scan 2804 with the inner thermoform model 2808 as shown in FIGS. 33A-33B. Forming an inner thermoform model 2808 over the sensor model 2812 confirms that there is enough space between sensor and occluding arch when outer retainer plastic is formed over sensor in the real world (to be described herein below). If there is not enough space, the occluding arch will bite into the outer retainer material and the sensor must be placed again, moved down towards the gingiva. One or models of a human's bite may be formed with the inner thermoform model 2808 as shown in FIGS. 34A-34B.

### Providing an Outer Thermoform Mold

With continued reference to FIG. 28, method 2800 includes, at step 2810 forming an outer thermoform model corresponding in shape to at least a portion of the lower set of teeth with a protrusion formed thereon. Outer thermoform model 2824 possess the same contours as at least a portion of the same teeth as inner thermoform model 2808, the plastic is formed with an offset matching in shape and size to an inner retainer, which will be affixed thereto to close the pocket in which the sensor is disposed and attached.

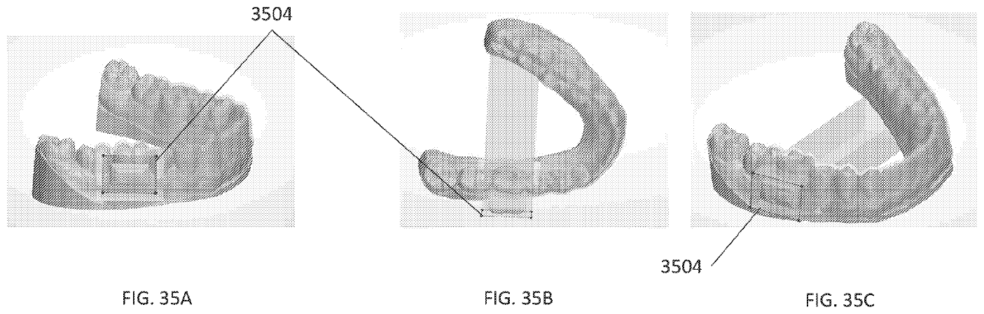

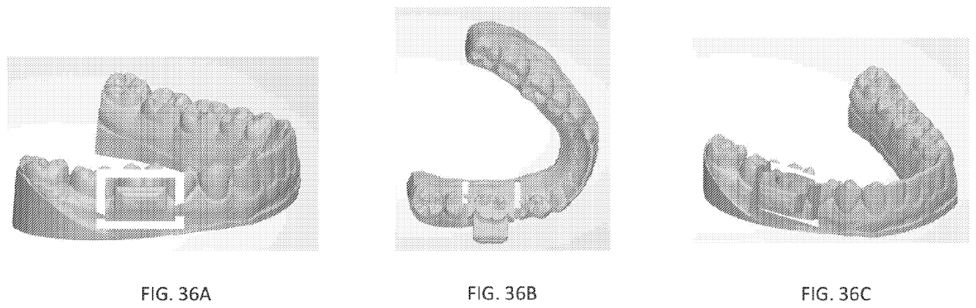

Forming the outer thermoform model 2824 further may include cutting inner thermoform model 2808 as shown in FIGS. 35A-35C. The outer thermoform model 2824 may include placing cut points 3504 circumscribing the sensor model 2812 on inner thermoform model 2808. The cut points 3504 may be offset from the sides of the sensor an equal amount, for example 1 mm orthogonally offset from the sides of sensor model 2812. After cut, outer thermoform model 2824 may be offset the exact specified distance from each side of the sensor model 2812, as specified by the cutout model as previously described. The sensor model 2812 may be oriented such that the top face of the sensor model 2812 is parallel and the sides are perpendicular to a screen on which a user is working, thereby ensuring a normal cut to the inner thermoform model 2808. Forming the outer thermoform model 2824 may include placing four cut points on the four inner corners of the cut caused by the cutout model, the process for which is shown in FIGS. 36A-36B.

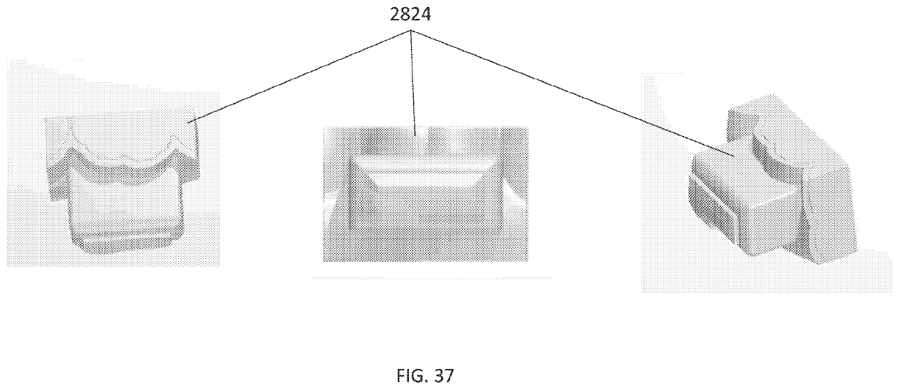

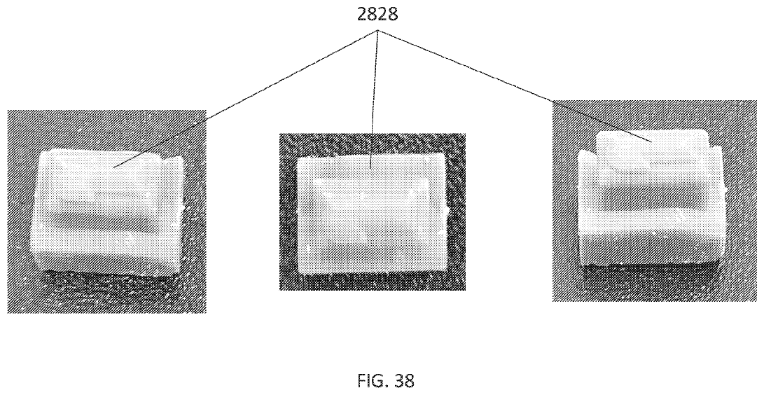

The outer thermoform model 2824 has at least a portion of the contours of teeth and gums, as shown in FIG. 37. The outer thermoform model 2824 may be formed with a flat or generally flat back or at least one generally flat side, shown in FIG. 37. Forming an outer thermoform mold 2828 may include 3D printing or otherwise additively manufacturing outer thermoform model 2824. Forming outer thermoform mold 2828 may include subtractively manufacturing the outer thermoform mold 2828 such as machining, turning, or otherwise cutting a stock material to final shape of outer thermoform model 2824. In various embodiments, a 3D printed outer thermoform mold 2828 is shown in FIG. 38. Thermoforming the Inner Retainer over the Inner Thermoform Mold

With continued reference to FIG. 28, method 2800 includes, at step 2815, thermoforming an inner retainer 2836 around the inner thermoform mold 2832, the inner retainer 2836 is configured to mount at least a portion of the lower set of teeth. Forming inner retainer 2836 includes thermoforming inner retainer 2836 utilizing a thermoforming machine.

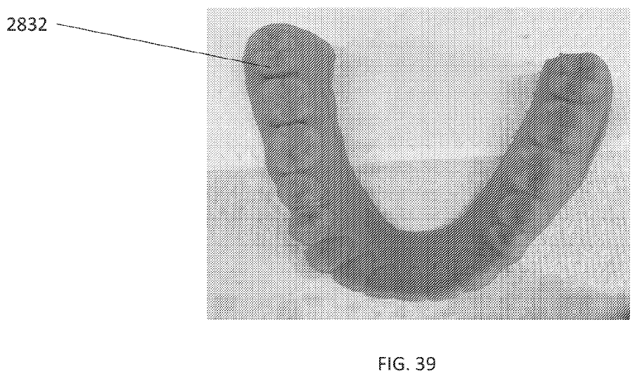

The inner thermoform mold 2832 has at least a portion of the contours of teeth and gums of inner thermoform model 2808 as shown in FIG. 39. The inner thermoform mold 2832 may be formed with a flat or generally flat back or at least one generally flat side. Forming inner thermoform mold 2832 may include 3D printing or otherwise additively manufacturing inner thermoform model 2808. Forming inner thermoform mold 2832 may include subtractively manufacturing the inner thermoform mold 2832 such as machining, turning, or otherwise cutting a stock material to final shape of inner thermoform model 2808. In various embodiments, a 3D printed inner thermoform mold 2832 is shown in FIG. 39.

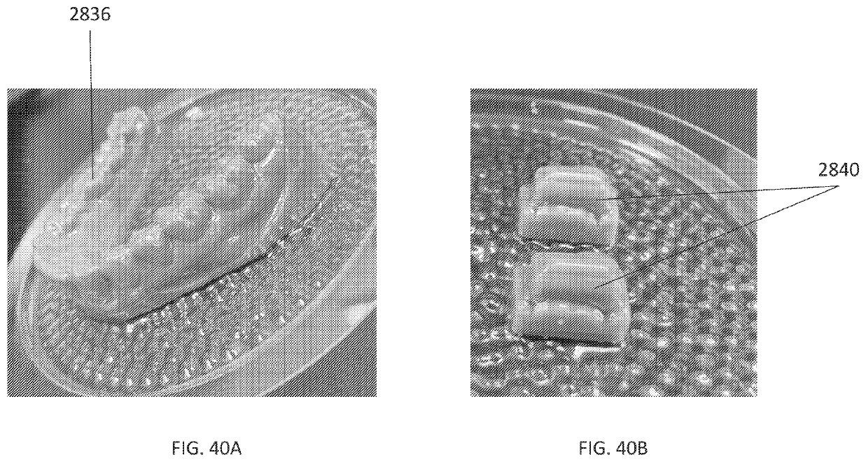

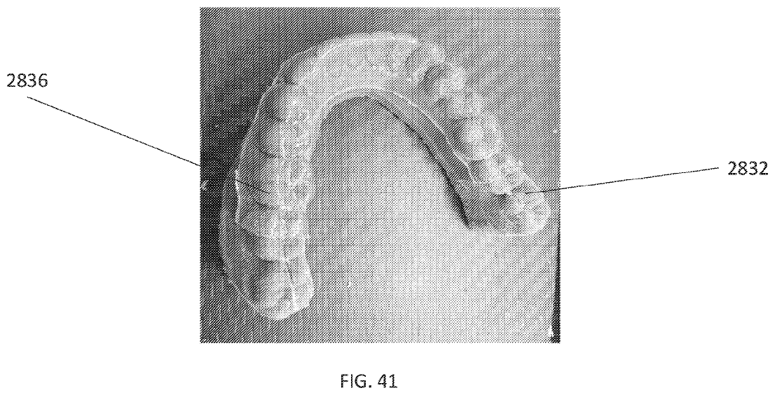

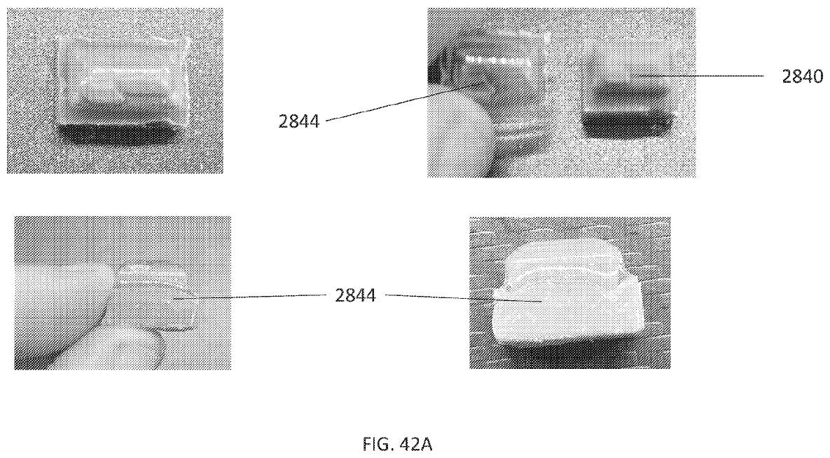

For the purposes of this disclosure, thermoforming may be the same or a similar process as described hereinabove. Thermoforming may be any process in which a pliable material is formed with a mold, shown in FIG. 40A. In various embodiments the plastic may be subjected to a pressure differential or vacuum to absorb shape of the models.

In various embodiments, small tabletop or lab size machines may be used to heat cut sections of plastic sheet and stretch it over a mold using vacuum.

Although the exemplary embodiment shown includes a polymeric retainer structure, the devices disclosed herein can be fabricated with one or more of many materials such as metal, glass, reinforced fibers, carbon fiber, composites, reinforced composites, aluminum, biological materials, and combinations thereof. The device can be shaped in many ways, such as with thermoforming or direct fabrication (e.g., 3D printing, or additive manufacturing). Additionally or alternatively, the retainers or portions thereof can be fabricated with machining such as an appliance fabricated from a block of material with computer numeric control machining.

The appliance can be designed specifically to accommodate the teeth of the user (e.g., the topography of the tooth receiving cavities matches the topography of the user's teeth), and may be fabricated based on positive or negative models of the user's teeth generated by impression, scanning, models, etc.

Alternatively, the appliance can be a generic device configured to receive the teeth, but not necessarily shaped to match the topography of the user's teeth. In some embodiments, only certain teeth received by the retainer structure will be repositioned while other teeth can provide a base or anchor region for holding the retainer structure in place as it applies force against the tooth or teeth targeted for repositioning.

### Trimming the Formed Retainers

Forming the inner retainer 2836 may include trimming a perimeter portion of the inner retainer 2836 formed after thermoforming. The plastic vacuum-shaped to the mold may be cut away and form a jagged edge, therefore trimming using hand tools or power tools to smooth and further refine the shape and edge of inner retainer 2836 to user's desire and comfort for the person on which the intraoral monitor is installed. All cuts to the plastic may be performed by lasers, milling machines, or any other automated process utilizing splines imported during the design process, such as the spline in reference to retainer model 2808 as described above. The plastic may be trimmed on inner retainer 2836 so the plastic only remains on the tooth surface area not on the other sides of the first base model.

### Thermoforming the Retainers

Forming inner retainer 2836 may include forming inner retainer 2836 having the contours of at least a portion of a lower set of teeth. Forming inner retainer 2836 may include forming inner retainer 2836 having the contours of at least a portion of an upper set of teeth. Forming inner retainer 2836 may include forming inner retainer 2836 having the contours of at least a portion of a full set of lower teeth and gums. Forming inner retainer 2836 may include forming inner retainer 2836 having the contours of at least a portion of a full set of upper teeth and gums. Forming inner retainer 2836 having an outer surface having a buccal side, an occlusal side and a lingual side. Forming inner retainer 2836 having an inner surface abutting and corresponding to in shape and contour of the teeth.

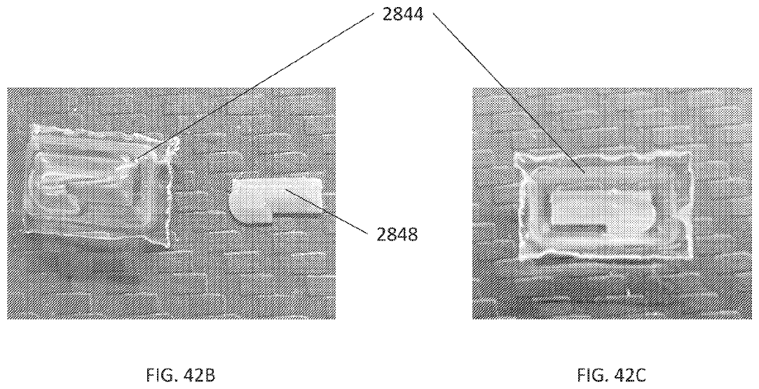

With continued reference to FIG. 28, method 2800 includes, at step 2820, thermoforming an outer retainer 2840 around the outer thermoform mold 2828. Outer retainer 2844 may be formed via a thermoforming machine as described hereinabove. Forming outer retainer 2840 includes forming a pocket 2844 disposed on the buccal side of the outer retainer 2840. The pocket 2844 may form the complimentary shape of the protrusion from outer thermoform mold 2840, the removal of which produces a negative space corresponding in shape to the sensor 244. Pocket 2844 protrudes through the outer surface of outer retainer 2840 on the buccal side, thereby forming a negative space on the inner surface of outer retainer 2840. Pocket 2844 is contiguous with the entirety of the outer retainer and formed from the same material and in the same step as forming outer retainer 2844. Pocket 2844 corresponds in contour, shape and size to the sensor 244 and formed there around. Pocket 2844 may include a protrusion configured to be removed in later steps. For example, an excess of plastic may be formed on the cutaway block 2815 transducer (buccal) side of the pocket 2844, the excess plastic formed over the cutaway block 2815 proximate the transducer of sensor 244 and shown in FIGS. 42A-42C.

### Positioning the Sensor in the Pocket of the Outer Retainer

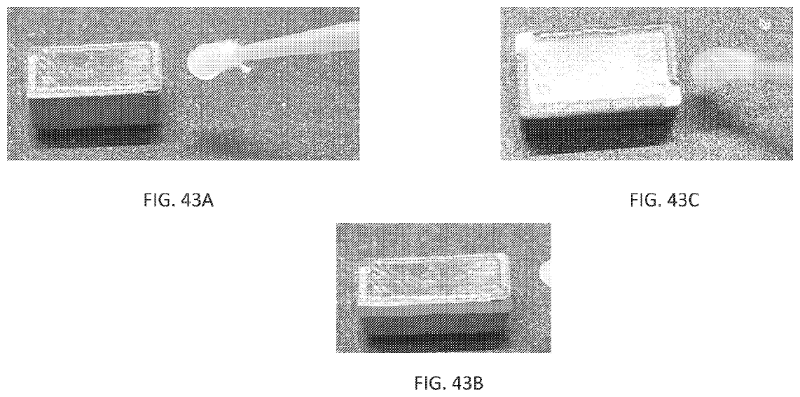

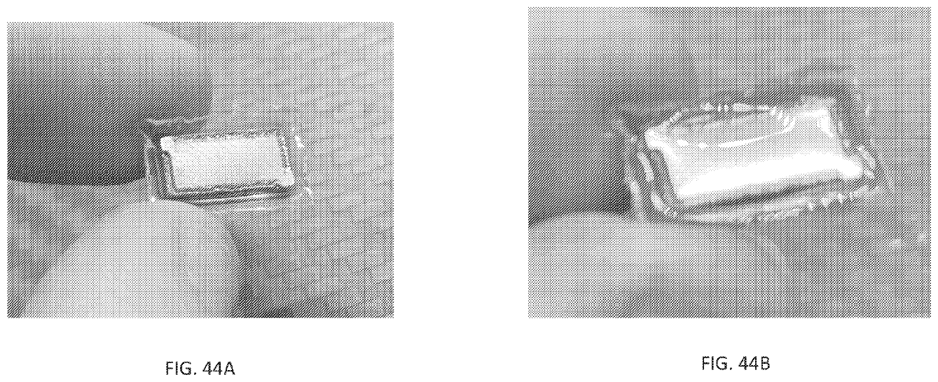

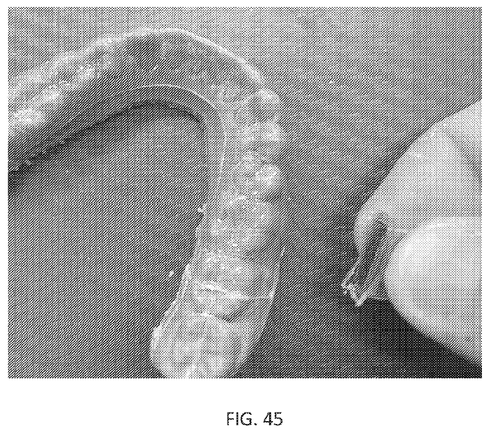

With continued reference to FIG. 28, method 2800 includes, at step 2825, positioning a sensor 244 in the pocket 2844 formed in outer retainer 2840. Note the sensor 244 is a physical sensor as opposed the sensor model 2812. Positioning the sensor 244 in the pocket 2844 includes applying an adhesive to the sensor 244 prior to inserting into the pocket 2844. Applying an adhesive to sensor 244 may be performed by a cotton swap manually as in FIGS. 43A and 43C. Applying an adhesive to sensor 244 may be applied on the edges of the sensor 244 as shown in FIG. 43B. Applying an adhesive to sensor 244 may include applying an adhesive to all sides of the sensor 244. In various embodiments, an adhesive may not be applied to the side of the sensor where the transducer is disposed, thus not introducing an adhesive to the sensitive portion of the device. In various embodiments, applying an adhesive to the sensor 244 includes applying UV an adhesive on walls of sensor to ⅓ or ½ way down the wall (shown in FIG. 43B). An adhesive is needed so that sensor box walls stick inside the retainer and form a barrier next to the sensor walls. The adhesive may be applied part way down so the adhesive does not run onto the transducer face of sensor 244, interfering with the cut-away post cap step or contaminating the transducer.

Positioning the sensor 244 into the pocket 2844 includes positioning the sensor 244 into pocket 240 transducer side first, thereby exposing an inner surface of the sensor 244 coincident with the inner surface of the outer retainer 236 as shown in FIG. 44A-44B. Coupling the Retainer Parts to enclose the Sensor

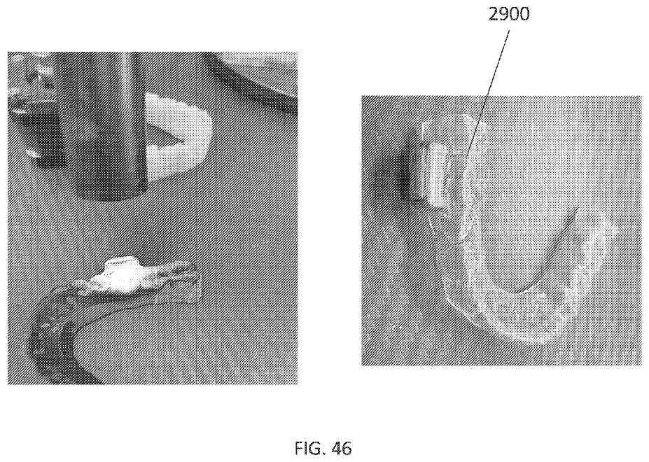

With continued reference to FIG. 28, method 2800 includes, at step 2835 coupling outer retainer 2840 to inner retainer 2836 by positioning outer retainer 2840 over the inner retainer 2836, thereby closing the pocket 2844. In various embodiments, aligning outer retainer 2840 over inner retainer 2836 includes aligning inner retainer 2836 with pocket 2844, thereby closing the pocket 2844. Coupling outer retainer 2840 to inner retainer 2836 may include applying an adhesive to the rear side of the sensor 244 prior to contacting the outer and inner retainers, thereby contacting the sensor 244 and the inner retainer 2836 with a layer of an adhesive, such as UV setting epoxy as describe herein. Aligning the outer retainer 2840 over inner retainer 2836 and exposing the adhesive therebetween to ultraviolet (UV) light, thereby curing the adhesive as in FIG. 46. In various embodiments, outer retainer 2840 and inner retainer 2836 may be welded via one or more lasers. In various embodiments, the laser may be automated or manually directed to the area to be welded. In various embodiments, a portion of each of the outer retainer 2840 and inner retainer 2836 may overlap, the overlapping sections disposed about the pocket in outer retainer 2840. In various embodiments, the overlapping portions of the outer retainer 2840 and inner retainer 2836 may be exposed to a laser beam, thereby welding the outer retainer 2840 and inner retainer 2836 together.

### Trimming a Portion of the Retainer

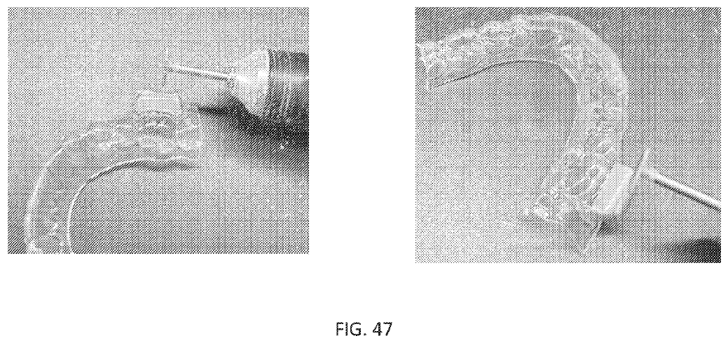

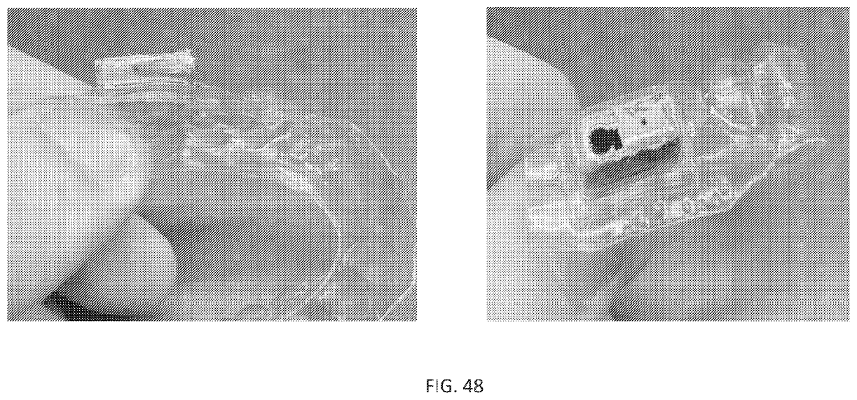

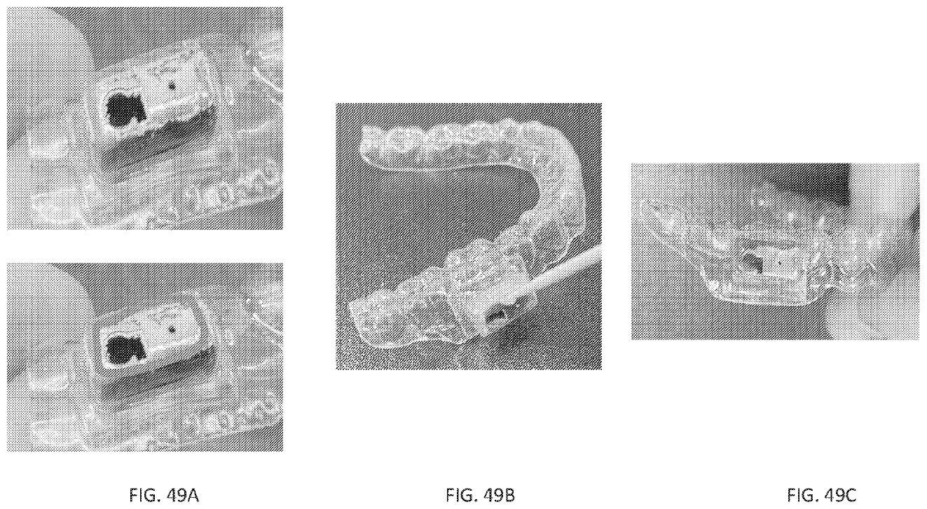

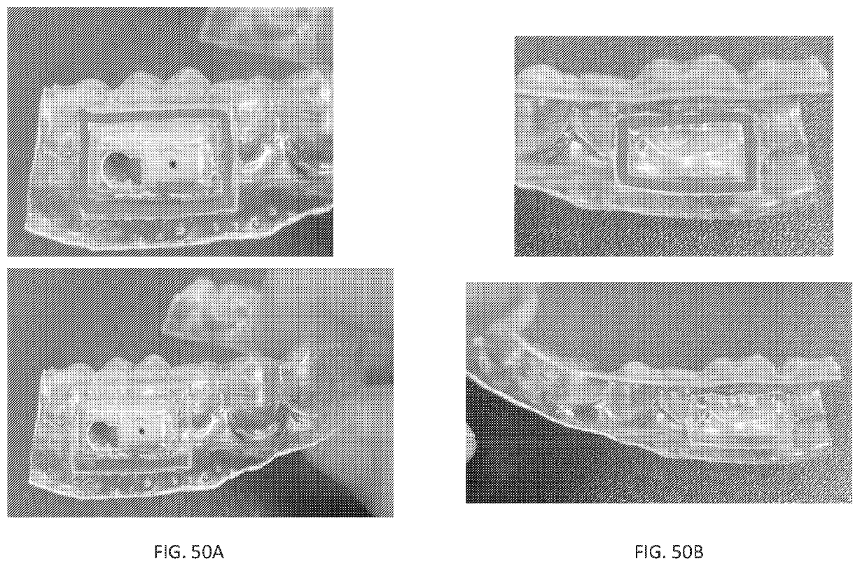

With continued reference to FIG. 28, method 2800, includes at step 2830, removing a portion of outer retainer 2840 proximate the sensor 244 to form an opening in the outer retainer 2840. In various embodiments, removing a portion of the outer retainer 2840 may include destructively removing a portion of the plastic via one or more saw, sawzalls, cutting wheels, grinders, knives, blades or another tool to cut the outer retainer 2840. In various embodiments, removing the portion of the outer retainer 2840 may include utilizing hand reciprocating, oscillating, rotating, or vibrating power tools to remove the portion of plastic as shown in FIG. 47. Removing the portion of outer retainer 2840 may include exposing a portion of the sensor 244 proximate the transducer as described herein, thereby exposing the transducer to the inside of the mouth of the human on which it is installed. The plastic of the outer retainer 2844 is therefore removed from the transducer side of the sensor 244 as shown in FIG. 48. There may be a seam formed between the cut edge of the portion of the outer retainer 2840 and sensor 244. An adhesive may be applied to the seam as shown in FIGS. 49A-49C. Applying an adhesive to the seam may include sealing the seam against the ingress of one or more contaminants, fluids, and portions of food or the like. Sealing the seam may include funneling saliva to the transducer of the sensor 244. In various embodiments, applying an adhesive to the seam may include applying an adhesive to the seam manually such as utilizing a toothpick or other implement with a relatively small tip to apply an adhesive to the seam area in FIG. 49B. In various embodiments, applying an adhesive to the seam may include applying the adhesive automatically through one or more automated processes or robotically.

### Method for 3D Printing an Intraoral Monitor

In various embodiments, a method for 3D printing an intraoral monitor includes forming an inner retainer model, positioning a sensor model and attaching the sensor model to the inner retainer model, cut the inner retainer model to form outer retainer model, cut outer retainer to form opening, combine the inner retainer model and the outer retainer model, and cut the sensor shell from the combined retainer model. The method may include 3D printing the retainer and the sensor shell, coating walls of sensor shell with adhesive, loading the sensor in the sensor shell, filling remaining volume of sensor shell with adhesive, filling sensor recess with adhesive, positioning the sensor shell on the sensor recess, applying glue to the seam between the sensor shell and the retainer, adding UV resin to the seams formed by the sensor and sensor shell on the transducer side of the sensor, and curing the retainer under UV light. In some embodiments, these steps are performed in the specific temporal sequence listed above. Additionally or alternatively, these steps can be performed in a varying order, with or without time gaps and/or intervening/intermediate steps therebetween.

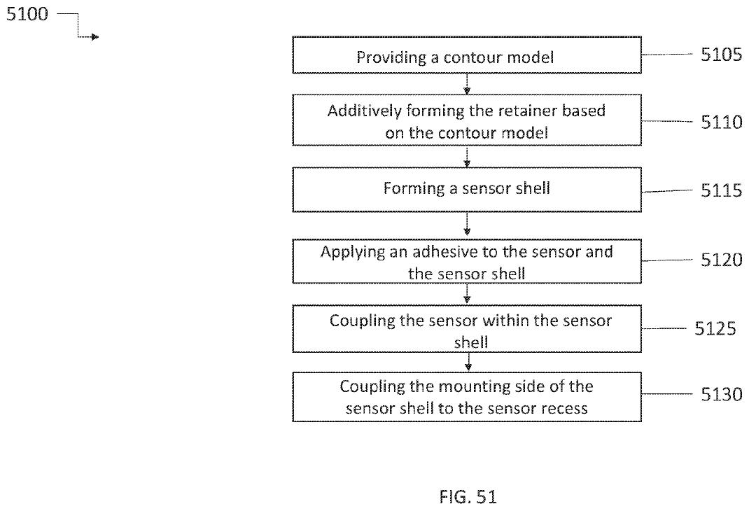

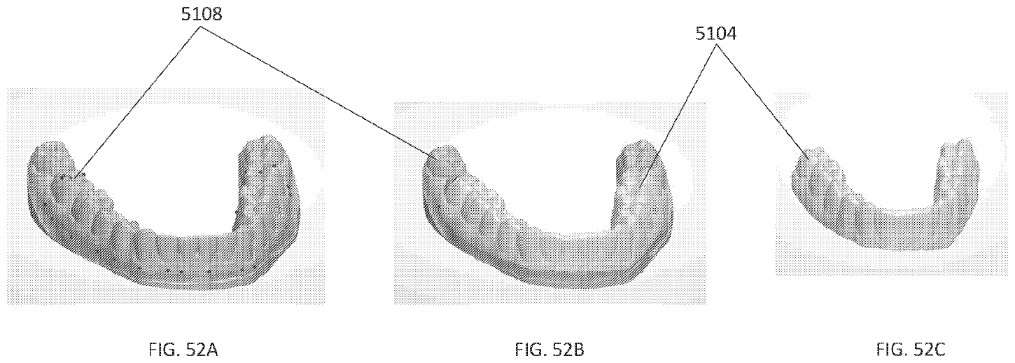

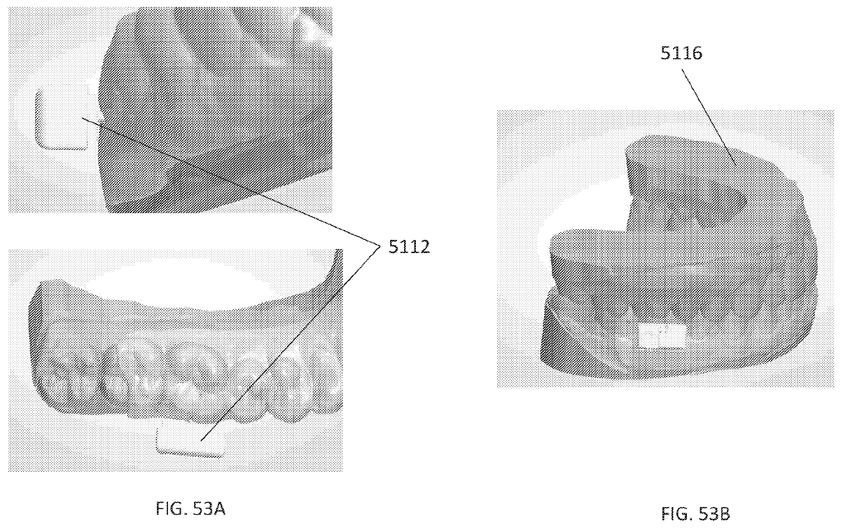

Referring now to FIG. 51, a method 5100 for forming an intraoral monitor is shown in flow diagram form. Method 5100 includes, at step S105, providing a contour model 5104, the counter model 5104 corresponding to at least a portion of a human's teeth and gums. Contour model 5104 may correspond to a scan 5108 of a human's teeth and gums as shown in FIGS. 52A-52C.

The contour model 5104 may be one or more computer models generated from 3D scans of at least a portion of a human's teeth and gums as shown in FIG. 52A-52c. A scan 5108 of one or more teeth of the patient may be performed. For example, a digital image (e.g. an STL/3D model of teeth) can be generated from an intraoral scanner (e.g. an intraoral scanner may form a portion of the intraoral monitor), such as in FIG. 52A-52B.

Manufacturing of these devices includes performing a three dimensional in-bite scan or modeling of the user's particular oral anatomy, and can be positioned to avoid interference with the user's bite as shown in FIG. 3C. Additionally, any conductive traces of the circuitry and printed circuit board (PCB) locations are outlined based on this preliminary scan. This step can be performed in isolation, e.g., without the material which forms the structure of the retainer being present. The absence of the retainer material is advantageous in that it maximizes the flexibility of the sensor design such that the placement and orientation of the sensor components can be customized to each user's anatomy to ensure there is no occlusion or contact with the opposing teeth, when the user has a closed bite.

In some embodiments, a CAD file of one or more of the patient's teeth is created, such as via an intraoral 3D scan and/or a 3D scan of a mold (e.g., an alginate mold) of the patient's teeth. The CAD file can be uploaded into memory storage of the patient's clinician (e.g., dentist). A customized intraoral monitor (e.g., or a portion of the device) can be manufactured for that particular patient based on the CAD file, and subsequently shipped to the patient and/or their clinician (e.g., based on address information similarly stored and associated with the CAD file). An alert can be sent to the patient and/or clinician related to delivery, and/or invoicing can automatically be triggered. In some embodiments, replacement devices can be similarly manufactured, delivered, and/or invoiced (e.g., without the need for one or more additional actions by the patient or clinician).

Further in reference to forming the model of a human's teeth, an occlusal model 5116 (showing a human's bite) may be performed to test the model of the retainer formed on the teeth model. Forming a scan 5108 of a person's teeth may include forming a contour model 5104 with one or more thicknesses, the contour model 5104 including the contours of the teeth. Forming the contour model 5104 may include design with a preselection of a retainer material, for example forming the contour model 5104 based on the material selection. Forming the contour model 5104 may include setting a spline to cut within the 3D modeling environment. The spline may correspond to the contours and topology of the teeth and gums proximate the retainer model. For the purposes of this disclosure, retainer, aligner, splint and other terminology for the device that sits on the teeth and holds the intraoral monitor (i.e., sensor) may be used interchangeably without departing from the scope of the disclosure.

### Positioning a Sensor Model

With continued reference to FIG. 51, forming scan 5108 of the human teeth includes positioning a 3D model of the sensor 5112 on the contour model 5104. In various embodiments, the sensor may be any sensor as described herein. In various embodiments, the sensor may be generally box-shape, with a top, bottom, left, right, front and back side with generally rounded edges on at least a subset of the edges formed thereon. The sensor model 5112 may include one or more positioning features as shown in FIGS. 53A-53B. In various embodiments, the sensor model 5112 may include one or more positioning features such as pegs, posts, boxes, standoffs, cases, or the like. In various embodiments, the sensor model may include one or more features designed to be removed once a tangible product is formed based on the model. Gingival-occlusal position of the sensor should be that the top (closest to occlusal plane) surface of the sensor should be slightly below occlusion from the maxillary arch, leaving slightly more space between occluding arch and top sensor surface than the thickness of the thermoformed plastic, that is to say that the retainer will terminate in the occlusal plane above the top surface of the sensor. In various embodiments, mesial-distal position of the sensor should be centered on the first or second molar, rotated so the sensor is in-line with the neighboring teeth. Gingival occlusal rotation should be so that top of sensor is close to contacting inner retainer on tooth but not quite, and close to contacting inner retainer on gums but not quite. One of ordinary skill in the art would appreciate that the figures and description of the positioning of the sensor are exemplary only, and provide only for a general description of the placement of the sensor. One of ordinary skill in the art would appreciate that an individual's teeth and mouth are unique, and may provide necessity to position the sensor accordingly.

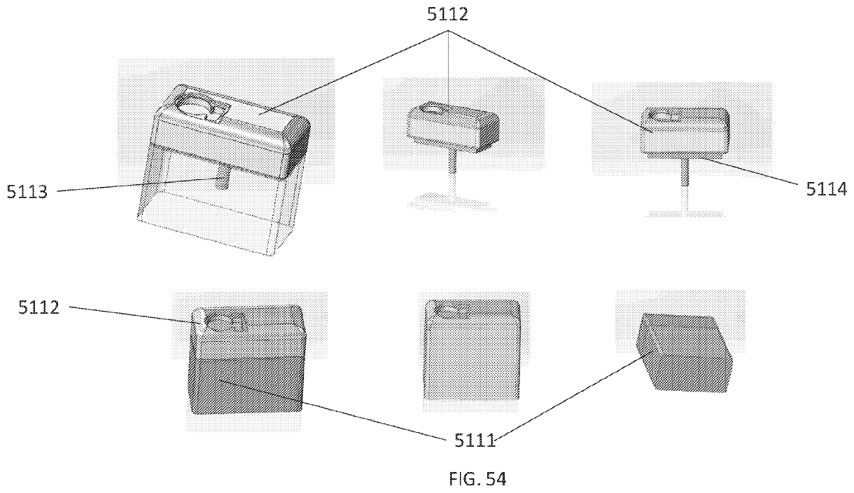

The sensor model 5112 may include one or more positioning features as shown in FIG. 54 as disclosed above. In various embodiments, the sensor model may include one or more positioning features such as pegs, posts, boxes, standoffs, cases, or the like. In various embodiments, the sensor model may include one or more features designed to be removed once a tangible product is formed based on the contour model 5104. As described herein, the sensor model 5112 may include geometry based on the sensor enclosure, a spacing block and a connector rod. In various embodiments, the spacing block ensures there is enough space between an inner retainer and sensor enclosure during positioning to account for tolerances. If too thin, sensor will hit inner retainer and there will be a gap between inner and outer retainer instead of flush. Spacing block can be for example 0-1 mm thick. In various embodiments, the spacing block may be 0.8 mm thick. For example, as described herein, the sensor model 5112 may include geometry based on the sensor enclosure, a spacing block 5114 and a connector rod 5113. In various embodiments, connector rod 5113 may be placed at the planar center of a bottom side of the sensor model 5112. Connector rod 5113 may be formed perpendicularly to the planar surface on which it is disposed. Connector rod 5113 may be configured to locate said sensor model 5112 within the retainer model 208, as shown in FIG. 3A-3B, wherein the connector rod punctures the retainer model 208. Connector rod 5113 may be a feature that can be laterally spaced or placed proximate any tooth in the model. In various embodiments, connector rod 5113 can be placed within a single tooth, between adjacent teeth, or through various teeth, according to the scan of the human's teeth and mouth, which are unique to the patient. In various embodiments, the spacing block 5114 ensures there is enough space between an inner retainer and sensor enclosure during positioning to account for tolerances. The spacing block 5114 provides a feature to space the sensor on the teeth and gums to account for lateral placement of the sensor in the occlusal plane such that when the spacing block 5114 contacts one or more contours of the teeth and gums, the sensor is placed appropriately for manufacturing of the physical retainer. In various embodiments the spacing block 5114 may have the same thickness as the selected plastic for thermoforming. In various embodiments, spacing block 5114 may have a smaller thickness than the thermoforming plastic such as to create a recess. In various embodiments, spacing block 5114 may have a larger thickness than the thermoforming plastic, thereby spacing the sensor model 5112 with excess room in a pocket. If too thin, sensor will hit inner retainer and there will be a gap between inner and outer retainer instead of flush. Spacing block 5114 can be for example 0-1 mm thick. In various embodiments, the spacing block 5114 may be 0.8 mm thick

The undercut block 5111 prevents thermoformed plastic from narrowing between sensor and an inner retainer, effectively narrowing the entrance into the pocket for, thereby preventing the sensor being loaded into the pocket as will be described below. The undercut block 5111 ensures that the model forming area where the sensor meets the retainer remains at least as wide as the sensor, as the opening of the pocket needs to be at least the height and width as the sensor to be loaded into the pocket. The undercut block 5111 may be configured to have the planform dimensions of the sensor model 5112, as shown in FIG. 54. In various embodiments, the walls of undercut block 5111 are thin enough so that the undercut model can be snapped off a third base model (as described herein below) when an outer retainer is removed, so the outer retainer plastic does not have to be twisted or pried off the undercut model, which can cause warping, amongst other deformities.

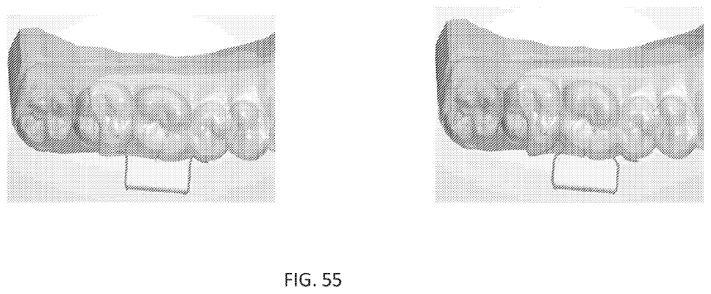

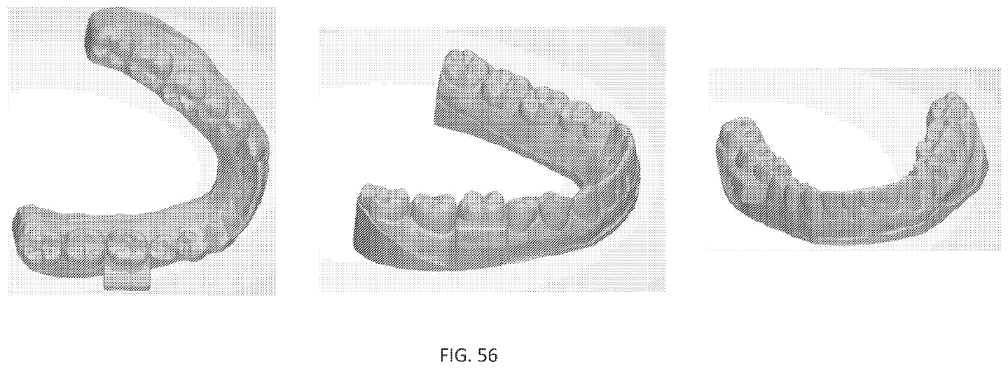

In various embodiments, providing a contour model 5104 includes digitally adding sensor model 5112 to the contour model 5104. The resultant contour model 5104 now includes the contours of the teeth and gums with the placed sensor model 5112 attached thereto. One or more users may then combine the scan 5108 with the contour model 5104 as shown in FIGS. 53A-53B, FIG. 55 and FIG. 56. Forming contour model 5104 over the sensor model 5112 confirms that there is enough space between sensor and occluding arch when outer retainer plastic is formed over sensor in the real world (to be described herein below). If there is not enough space, the occluding arch will bite into the outer retainer material and the sensor must be placed again, moved down towards the gingiva.

### Cutting the Sensor Shell and Openings in the Model

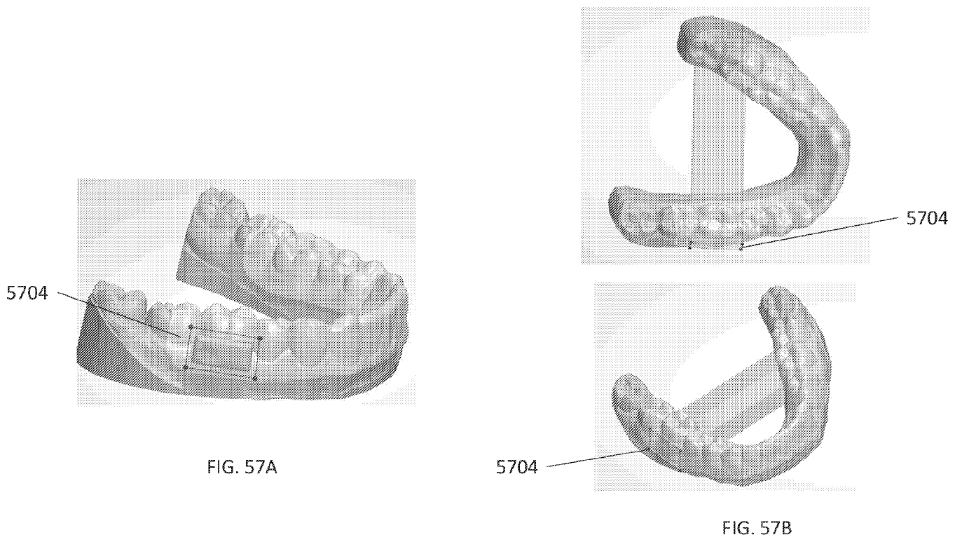

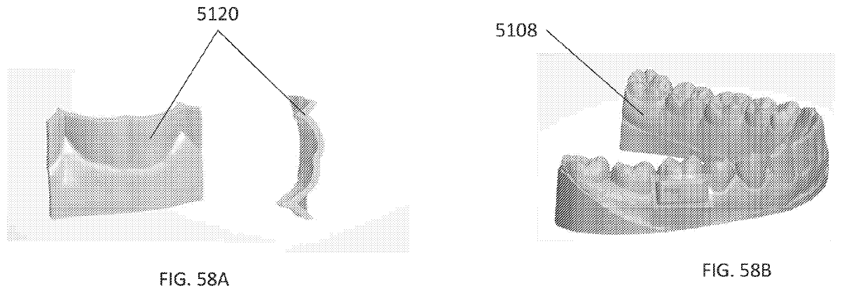

Forming contour model 5104 may include forming a cut in contour model 5104 and scan 5108 as in FIGS. 57A-57B. Forming the cut may include drawing a planform cut 5704, as the rectangular cut 5704 shown in FIG. 57A, and extruding that cut through the model and scan. The resultant cut from the operation may produce partial contour model 5120. Partial contour model 5120 may include the same contours of contour model 5104 from within the cut boundaries in FIGS. 57A-57B. An interference check can be performed in FIG. 58B, wherein the partial contour model 5120 circumscribes the scan area around sensor model 5112. Partial contour model 5120 may include a projected boundary of 1-2 mm around the sensor model 5112. The size of planform cut 5704 should be such that the inner retainer overlaps the later-formed outer retainer and no gaps remain. If offset distance needs to be more precise, distance can also be set using cutout model in sensor assembly, similar to in thermoform techniques

Forming the contour model 5104 further includes forming the contour model 5104 around scan 5108 with sensor model 5112 adequately located thereon the buccal side. One or more splines can be formed on scan 5108 to provide the eventual boundary for the retainer. The contour model 5104 may include one or more thicknesses selected for the area of the mouth it will be mounted on, the material of the retainer to be formed, and the like. Forming the contour model 5104 further includes cutting a portion of the contour

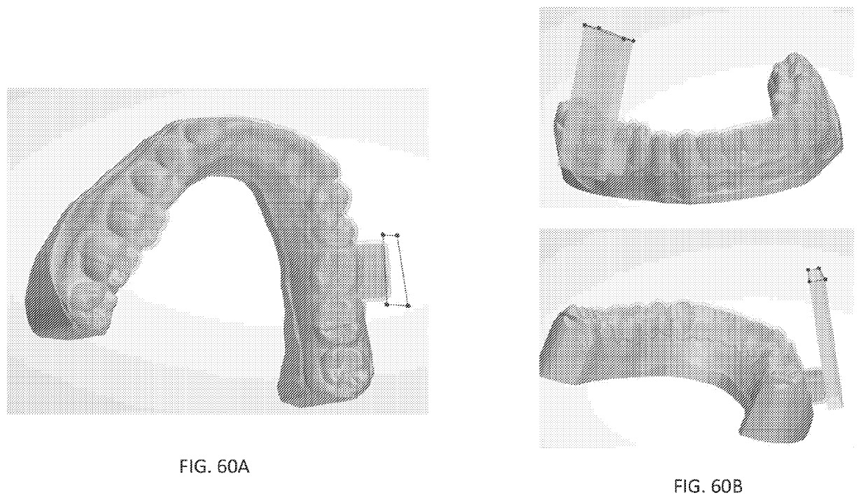

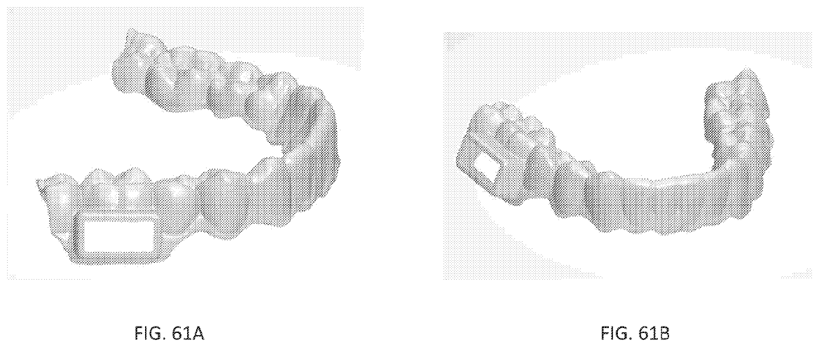

model 5104 proximate to the sensor contours that correspond to the eventual location of the transducer of the sensor. A cut may be made transverse to the face to be removed, such that the cut is performed parallel to said face and across, cutting away a generally rectangular opening in the contour model 5104. The placement and extrusion of cut can be seen in FIGS. 60A-60B, wherein the contour model 5104 is rotated so the top surface of the sensor portion of the contour model is in plane with the page, and a cut is made perpendicular to the transducer side of the sensor portion of contour model 5104. The resultant opening of contour model 5104 is shown in FIGS. 61A-61B.

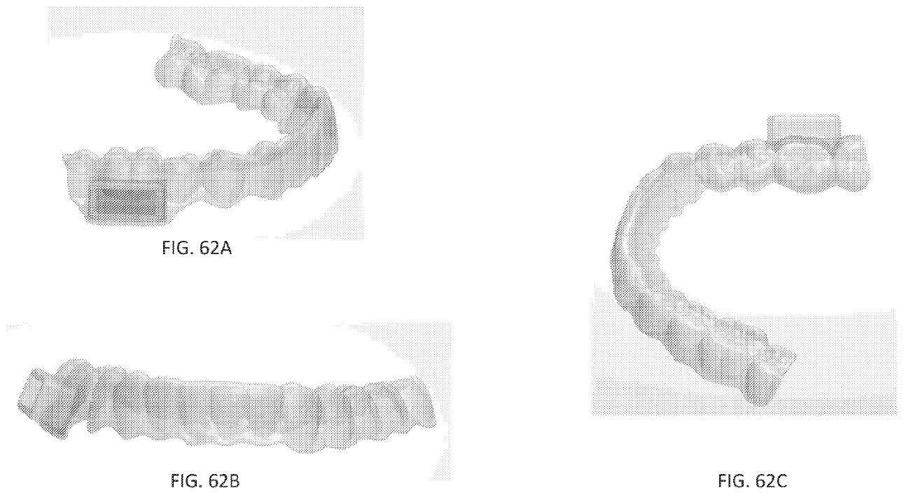

Forming contour model 5104 further includes adding the partial contour model 5120 to contour model 5104 as shown in FIGS. 62A-62C. The partial contour model 5120 overlaying perfectly the geometry of contour model 5104 directly to the lingual side of the sensor portion. Forming the contour model 5104 further includes cutting a sensor shell model 5124 from contour model 5104, the sensor shell having a top, bottom, left, right, transducer and mounting side, forming an open cavity therebetween, and a pass through from the transducer to the mounting side. The sensor shell model 5124 corresponds in internal dimension to sensor model 5112, configured, once manufactured, to partially enclose a sensor (244) therewithin. The cut that produced sensor shell model 5124 produces sensor recess model 5128 on the contour model 5104, the sensor recess retaining a portion of the walls of sensor shell model 5124, namely the top, bottom, left and right walls. The buccal face of these walls corresponds to the shape of the mounting side of the sensor shell. The sensor recess is bounded on the lingual side by the contour model 5104 contours of the teeth and gums, forming a shallow hollow. In various embodiments, the walls of sensor recess model 5128 and the walls of sensor shell model 5124 may include positioning or retaining structures, configured to complementarily couple to align said components.

### 3D Printing the Sensor Shell and the Retainer

With continued reference to FIG. 51, method 5100 includes, at step S110, additively forming a retainer 5132 based on the contour model 5104. In various embodiments, 3D printing retainer 5132. For the purposes of this disclosure, 3D printing may be any operation in which material is added in one or more layers and directions to form the workpiece. In various embodiments, retainer 5132 may be formed from one or more plastics. In various embodiments, retainer 5132 may be formed from one or more dental resins. In various embodiments a first portion of retainer 5132 may be formed from a first material as described herein and a second portion of the retainer 5132 may be formed from a second material as described herein. In various embodiments retainer 5132 may be formed from two or more types of plastic. In various embodiments, retainer 5132 may be formed from two or more types of dental resin. In various embodiments, retainer 5132 may be formed from stereolithography (SLA). In various embodiments, retainer 5132 may be formed from selective laser sintering (SLS). In various embodiments, retainer 5132 may be formed from fused deposition modeling (FDM). In various embodiments, retainer 5132 may be formed from digital light process (DLP). In various embodiments, retainer 5132 may be formed from multi jet fusion (MJF). In various embodiments, retainer 5132 may be formed from a polyjet process. In various embodiments, retainer 5132 may be formed from direct metal laser sintering (DMLS). In various embodiments, retainer 5132 may be formed from electron beam melting (EBM). In various embodiments, forming retainer 5132 includes forming the contour model 5104. In various embodiments, forming retainer 5132 includes importing contour model 5104. In various embodiments, forming retainer 5132 may include providing contour model 5104. In various embodiments, forming the retainer 5132 may include scanning a human's mouth for the contours of the teeth and gums as described herein. In various embodiments, forming retainer 5132 includes importing a stored scan 5108. In various embodiments, forming retainer 5132 includes translating any model described herein to computer-readable instructions for one or more automated manufacturing processes, such as 3D printing or milling machines (including 5 or 6 axis milling machines utilizing computer numerical control (CNC)).

In various embodiments, retainer 5132 may be formed from subtractive manufacturing processes or supplemented with subtractively manufacturing process. In various embodiments, retainer 5132 may be 3D printed and then finished in a milling machine to trim the perimeter of burrs and jagged edges. In various embodiments, retainer 5132 may be formed wholly in a milling machine automatedly, machining plastic or metals from a larger stock, removing material to form retainer 5132. Forming retainer 5132 includes forming sensor recess 5136 continuously with retainer 5132. In various embodiments, sensor recess 5126 is formed simultaneously and as one continuous workpiece with retainer 5132. In various embodiments, sensor recess is formed separately and coupled to retainer 5132 with one or more adhesives, lasers, geometrical mating features, or a combination thereof. In various embodiments, sensor recess 5136 is formed by removing material from retainer 5132, for example, retainer 5132 may be formed with a frangible portion disposed over sensor recess 5136, after manufacturing, a user or a machine may then cut or remove the frangible portion to reveal sensor recess 5136. In various embodiments, sensor recess 5136 is formed from the same material as retainer 5132. In various embodiments, sensor recess 5136 is formed from a distinct material than retainer 5132. For example and without limitation, sensor recess 5136 may be formed from a metal that is mounted to a plastic version of retainer 5132.

Forming retainer 5132 includes forming retainer 5132 having an outer surface having a buccal side, an occlusal side and a lingual side. Forming retainer 5132 may have an inner surface directly abutting and corresponding in shape and contour to the teeth. The retainer 5132 may be any retainer as described herein, and may have a buccal side proximate the cheek or lips of a user, an occlusal side proximate the opposite set of teeth and a lingual side proximate the tongue.

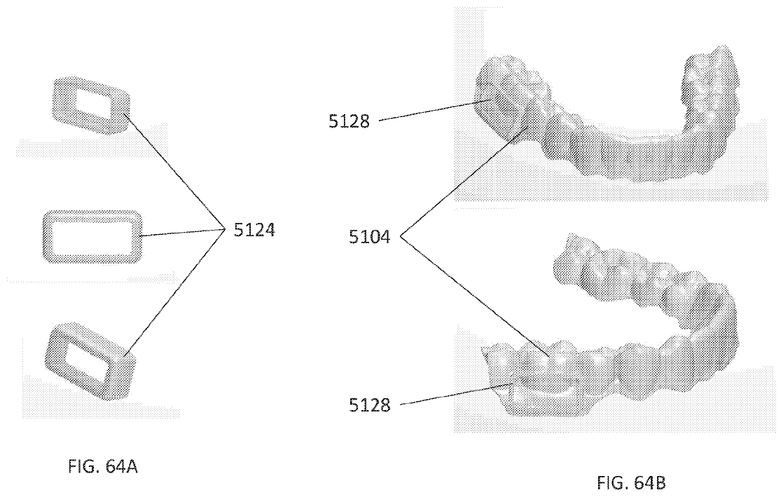

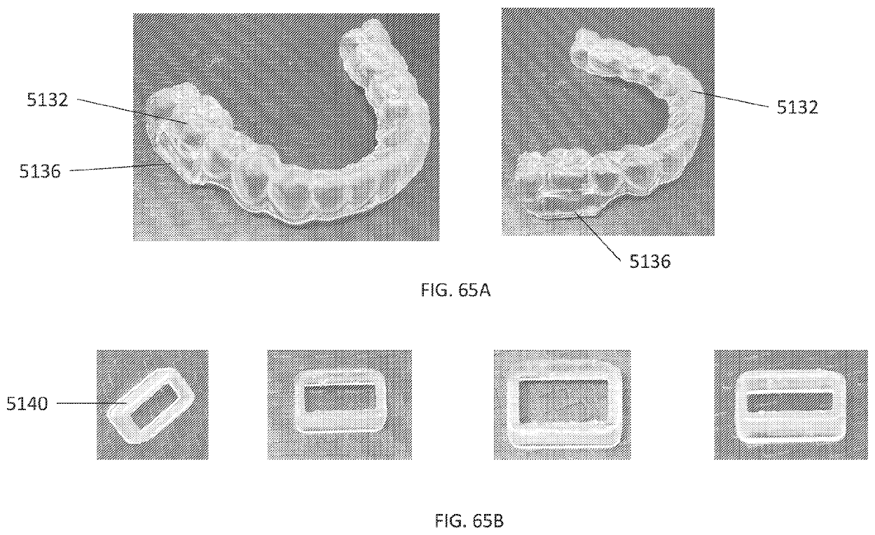

With continued reference to FIG. 51, method 5100 includes, at step S115, forming a sensor shell 5140, the sensor shell 5140 including a mounting side configured mount on the sensor recess 5136. Sensor shell 5140 may include the exact geometry of sensor shell model 5124. For example, sensor shell 5140 may include a top, bottom, left, right, transducer and mounting sides. The mounting and transducer sides may be opposite an in parallel planes, likewise the top and bottom sides may be opposite and parallel, connected therebetween at terminating ends by the left and right side walls, similarly opposite and parallel. The mounting side of sensor shell 5140 may include no wall at all, rather it is formed by the circumscribed edge of the top, bottom, left and right walls, as can be seen in FIG. 64A (showing the mounting side of the sensor shell model 5124).

In various embodiments, sensor shell 5140 may be formed from one or more plastics. In various embodiments, sensor shell 5140 may be formed from one or more dental resins. In various embodiments a first portion of sensor shell 5140 may be formed from a first material as described herein and a second portion of the sensor shell 5140 may be formed from a second material as described herein. In various embodiments sensor shell 5140 may be formed from two or more types of plastic. In various embodiments, sensor shell 5140 may be formed from two or more types of dental resin. In various embodiments, sensor shell 5140 may be formed from stereolithography (SLA). In various embodiments, sensor shell 5140 may be formed from selective laser sintering (SLS). In various embodiments, sensor shell 5140 may be formed from fused deposition modeling (FDM). In various embodiments, sensor shell 5140 may be formed from digital light process (DLP). In various embodiments, sensor shell 5140 may be formed from multi jet fusion (MJF). In various embodiments, sensor shell 5140 may be formed from a polyjet process. In various embodiments, sensor shell 5140 may be formed from direct metal laser sintering (DMLS). In various embodiments, sensor shell 5140 may be formed from electron beam melting (EBM). In various embodiments, forming sensor shell 5140 includes forming the sensor shell model 5124. In various embodiments, forming sensor shell 5140 includes importing sensor shell model 5124. In various embodiments, forming sensor shell 5140 may include providing sensor shell model 5124.

### Coupling the Sensor Shell to the Retainer

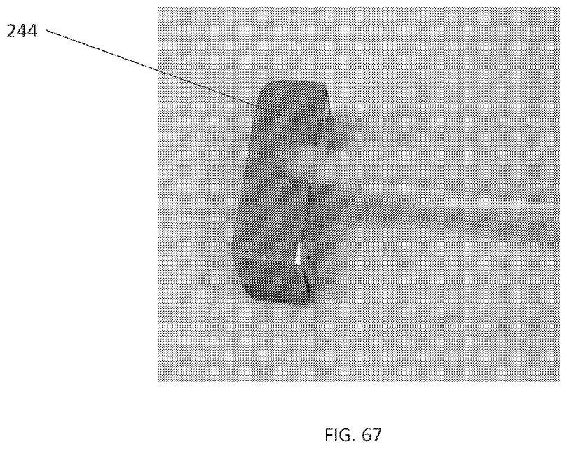

With continued reference to FIG. 51, method 5100 includes applying an adhesive to the sensor 244 and the sensor shell 5140. Applying an adhesive to sensor 244 may be performed by a cotton swap manually as in FIG. 67. Applying an adhesive to sensor 244 may be applied on the edges of the sensor 244. Applying an adhesive to sensor 244 may include applying an adhesive to all sides of the sensor 244. In various embodiments, an adhesive may not be applied to the side of the sensor where the transducer is disposed, thus not introducing an adhesive to the sensitive portion of the device. In various embodiments, applying an adhesive to the sensor 244 includes applying UV an adhesive on walls of sensor to ⅓ or ½ way down the wall. An adhesive is needed so that sensor box walls stick inside the retainer and form a barrier next to the sensor walls. The adhesive may be applied part way down so the adhesive does not run onto the transducer face of sensor 244, interfering with the cut-away post cap step or contaminating the transducer. Applying adhesive to the sensor shell may include applying adhesive to the top, bottom, left and right walls of sensor shell 5140, thereby forming adhesive on all contacting surfaces between the sensor 244 and the sensor shell 5140.

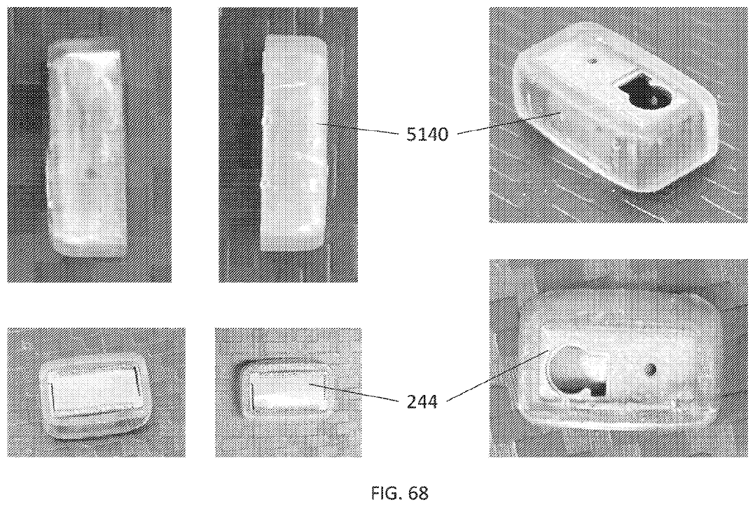

With continued reference to FIG. 51, method 5100 includes, at step S125, coupling the sensor 244 within the sensor shell 5140, as shown in FIG. 68, wherein the sensor is pushed fully within sensor shell 5140, the back side of the sensor 244 sitting flush or within the mounting side of sensor shell 5140. The sensor 244 has a transducer side that sits within the opening formed in the sensor shell 5140, shown in FIG. 68. Curing the adhesive between the sensor shell 5140 and the sensor 244 may include exposing the adhesive to ultraviolet light consistent with the disclosure of UV light exposure in the entirety of this disclosure.

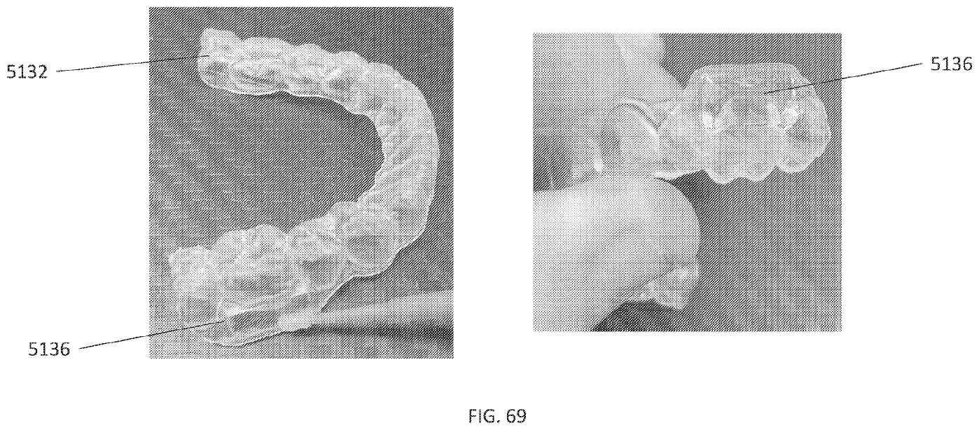

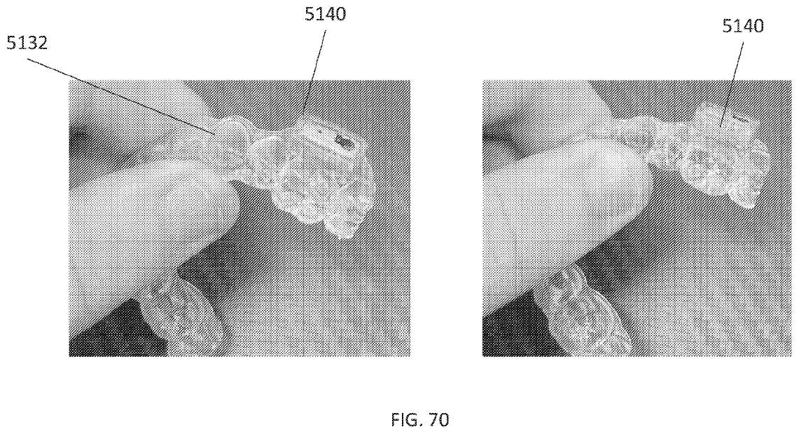

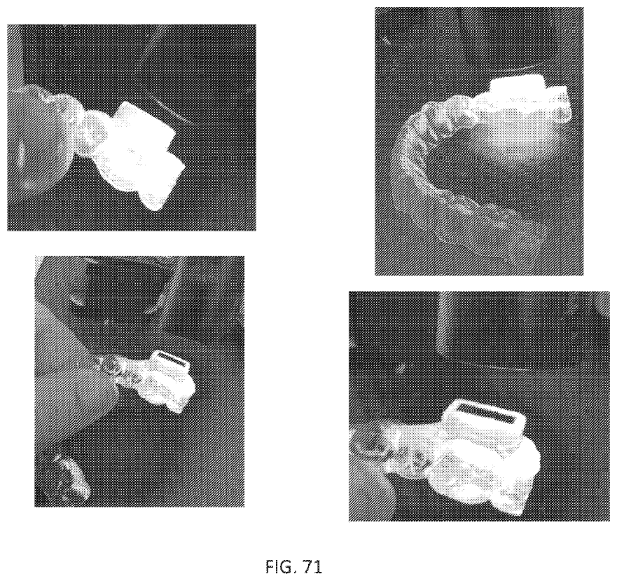

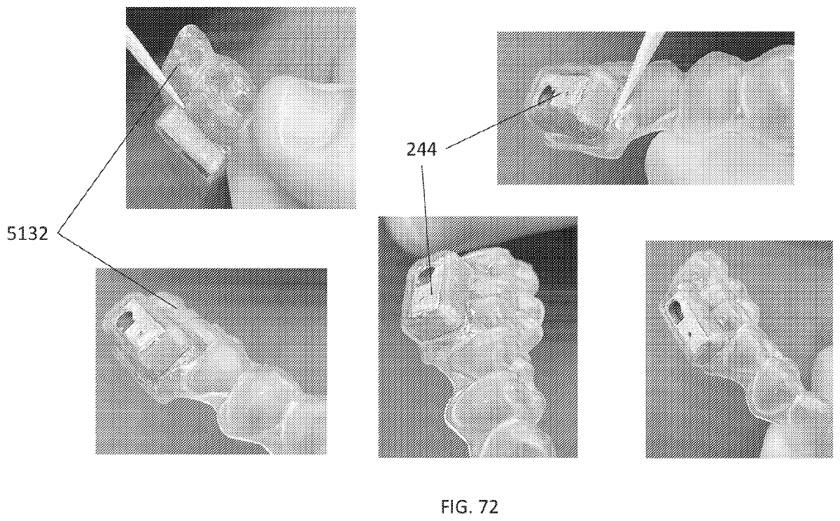

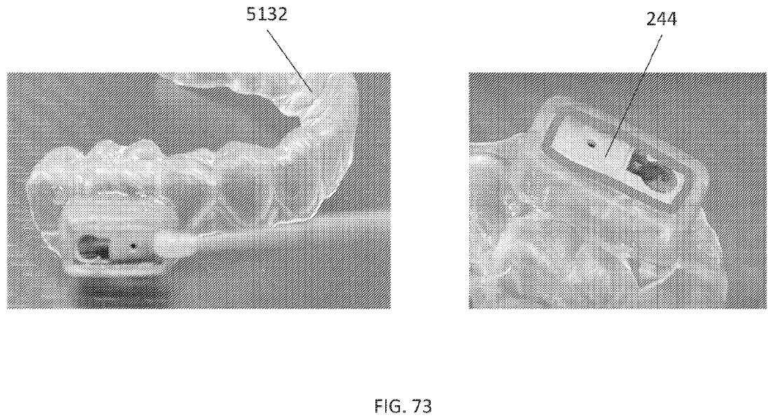

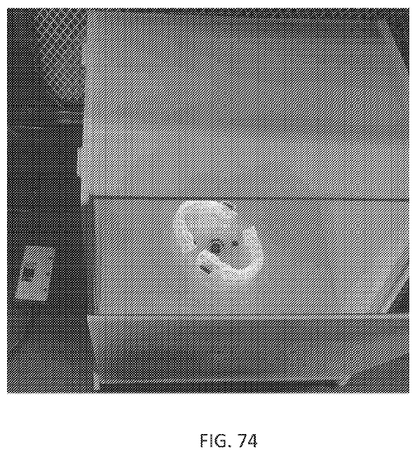

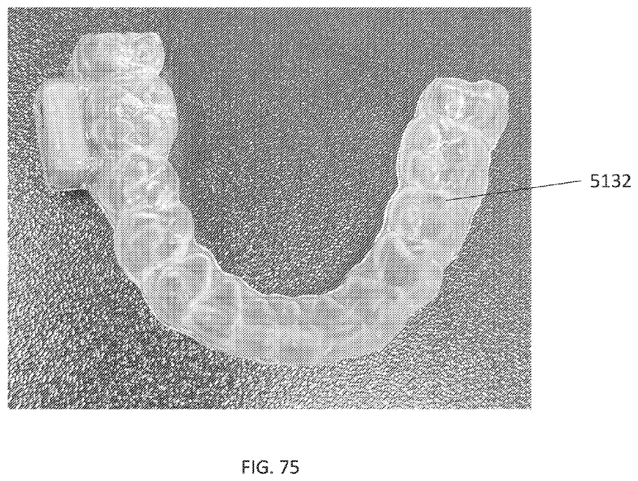

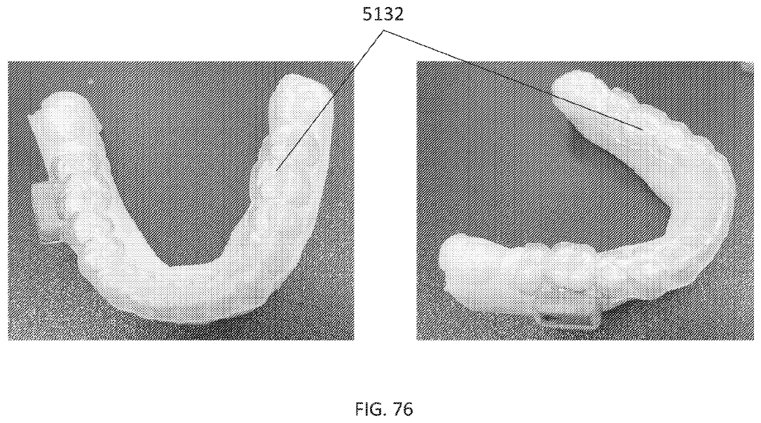

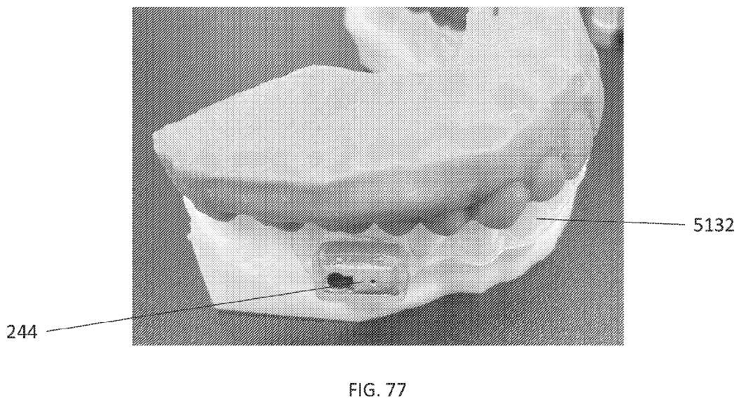

With continued reference to FIG. 51, method 5100 includes, at step S130, coupling the mounting side of the sensor shell 5140 to the sensor recess 5136. The adhesive may be applied to the back side of the sensor that sits flush with the mounting side of the sensor shell 5140. Coupling the mounting side of the sensor shell 5140 to the sensor recess 5136 may include applying glue to a seam formed by the sensor 244 and the sensor shell 5140 and the sensor shell 5140 and the retainer 5132, as shown in FIGS. 73 and 69, respectively. There may be a seam formed between the edge of the portion of the sensor shell 5140 and sensor 244. An adhesive may be applied to the seam as shown in FIG. 73. Applying an adhesive to the seam may include sealing the seam against the ingress of one or more contaminants, fluids, and portions of food or the like. Sealing the seam may include funneling saliva to the transducer of the sensor 244. In various embodiments, applying an adhesive to the seam may include applying an adhesive to the seam manually such as utilizing a toothpick or other implement with a relatively small tip to apply an adhesive to the seam area in FIGS. 69 and 73. In various embodiments, applying an adhesive to the seam may include applying the adhesive automatically through one or more automated processes or robotically. Curing the adhesive between the sensor shell 5140 and the retainer 5132 may include exposing the adhesive to ultraviolet light consistent with the disclosure of UV light exposure in the entirety of this disclosure and shown in FIG. 71. A final fit check may be performed with an occlusal model as shown in FIG. 77.

### A Method for 3D Printing an Intraoral Monitor

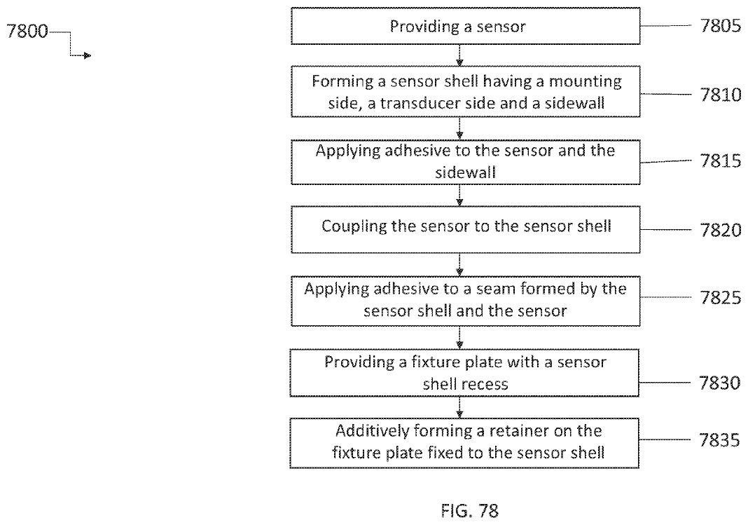

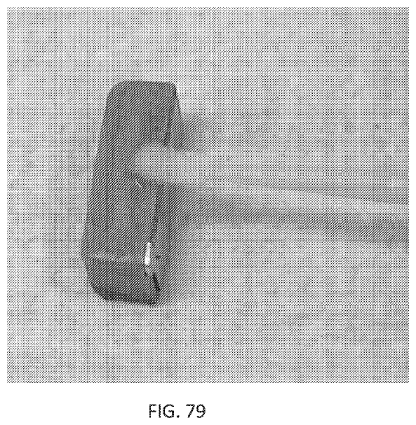

Referring now to FIG. 78, a method 7800 for forming an intraoral monitor is presented in flow diagram form. The method 7800 includes, at step 7805, providing a sensor 244. The sensor may be any sensor as described herein and as shown in FIG. 79.

In various embodiments, a method for 3D printing an intraoral monitor includes forming an inner retainer model, positioning a sensor model and attaching the sensor model to the inner retainer model, cut the inner retainer model to form outer retainer model, cut outer retainer to form opening, combine the inner retainer model and the outer retainer model, and cut the sensor shell from the combined retainer model. The method may include 3D printing the sensor shell, coating the sidewalls of the sensor shell with adhesive, loading the sensor in the sensor shell, adding UV resin to the seams on the transducer face of the sensor and sensor shell, loading the sensor shell into the fixture plate, 3D printing retainer on the fixture plate, ejecting retainer from fixture plate, and curing the retainer under UV light. In some embodiments, these steps are performed in the specific temporal sequence listed above. Additionally or alternatively, these steps can be performed in a varying order, with or without time gaps and/or intervening/intermediate steps therebetween.

### Forming the Sensor Shell

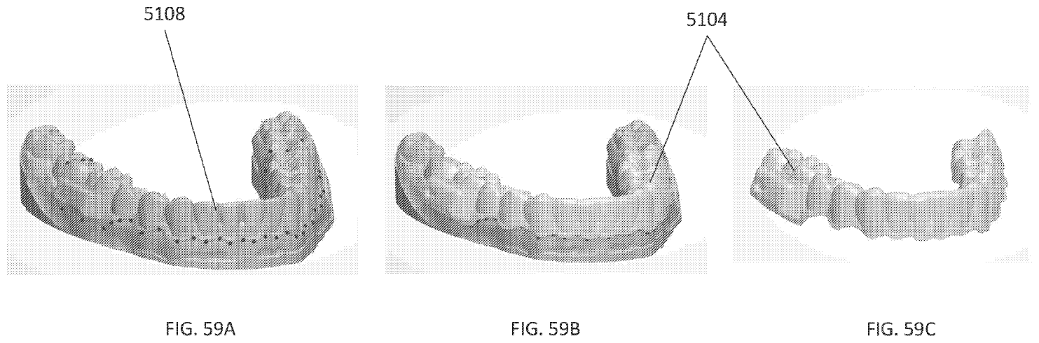

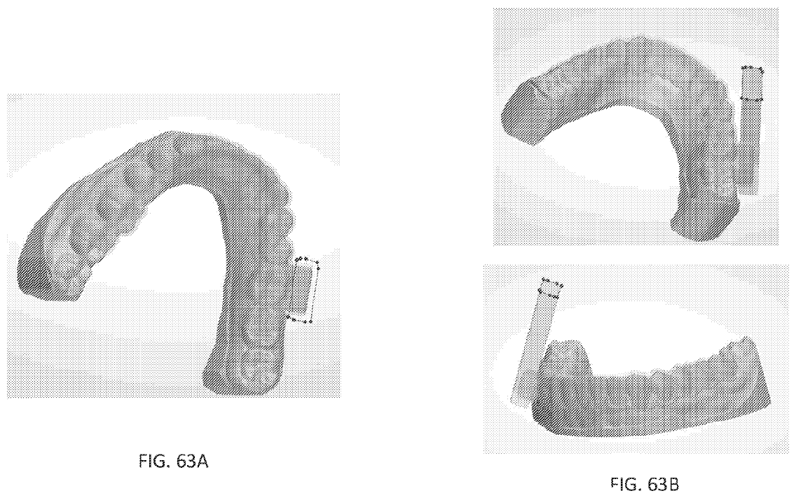

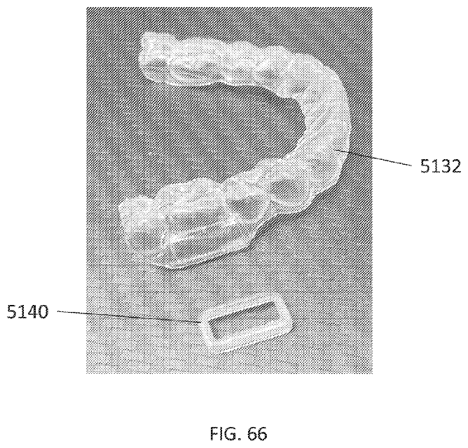

With continued reference to FIG. 78, method 7800 includes, at step 7810, forming a sensor shell, the sensor shell configured to mount onto the sensor and wherein the sensor shell further comprises a mounting side, a transducer opening side and at least one sidewall connected therebetween. The sensor shell 7804 may be formed in the same manner as sensor shell 5140 in reference to FIG. 51 and FIGS. 54-66. The sensor shell having the internal geometry to couple with sensor 244. The sensor shell 7804 may be formed from one or more plastics, dental resins or other suitable material. Sensor shell may be 3D printed or additively manufactured, as described herein. Sensor shell 7804 may be machined, cut, drilled or turned on subtractive manufacturing machines, automatedly or manually, in various embodiments.

### Coupling the Sensor and the Sensor Shell

With continued reference to FIG. 78, method 7800 includes, at step 7815, applying adhesive to the sensor shell 7804 and the sensor 244. The adhesive may be applied on the at least one sidewall of the sensor shell 7804. Applying an adhesive to sensor 244 may be performed by a cotton swap manually as in FIG. 79. Applying an adhesive to sensor 244 may be applied on the edges of the sensor 244. Applying an adhesive to sensor 244 may include applying an adhesive to all sides of the sensor 244. In various embodiments, an adhesive may not be applied to the side of the sensor where the transducer is disposed, thus not introducing an adhesive to the sensitive portion of the device. In various embodiments, applying an adhesive to the sensor 244 includes applying UV an adhesive on walls of sensor to ⅓ or ½ way down the wall. An adhesive is needed so that sensor box walls stick inside the retainer and form a barrier next to the sensor walls. The adhesive may be applied part way down so the adhesive does not run onto the transducer face of sensor 244, interfering with the cut-away post cap step or contaminating the transducer. Applying adhesive to the sensor shell may include applying adhesive to the top, bottom, left and right walls of sensor shell 7804, thereby forming adhesive on all contacting surfaces between the sensor 244 and the sensor shell 7804.

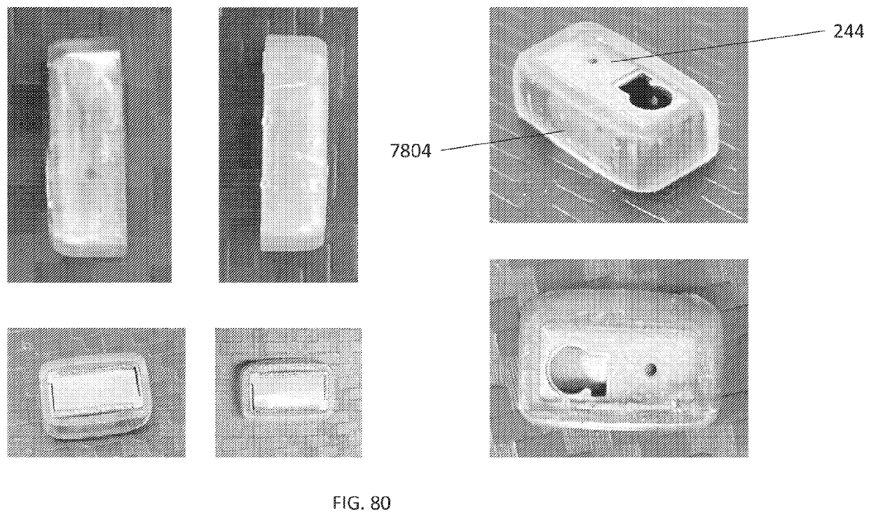

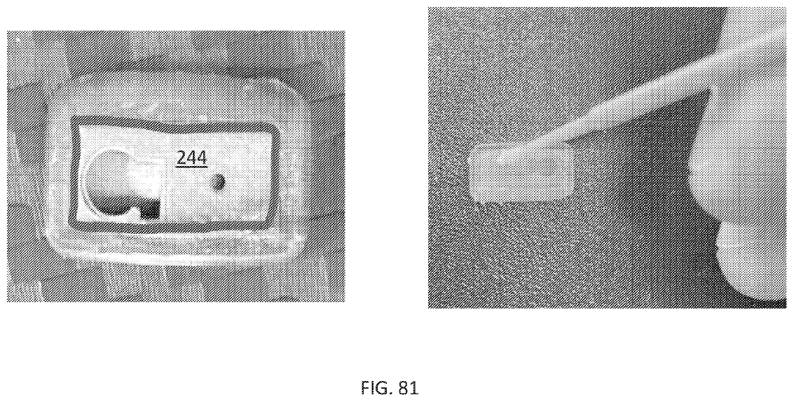

With continued reference to FIG. 78, method 7800 includes, at step 7820, coupling the sensor 244 to the sensor shell 7804. Coupling the sensor 244 within the sensor shell 7804, as shown in FIG. 80, wherein the sensor is pushed fully within sensor shell 7804, the back side of the sensor 244 sitting flush or within the mounting side of sensor shell 7804. The sensor 244 has a transducer side that sits within the opening formed in the sensor shell 7804, shown in FIG. 80. Curing the adhesive between the sensor shell 7804 and the sensor 244 may include exposing the adhesive to ultraviolet light consistent with the disclosure of UV light exposure in the entirety of this disclosure.

With continued reference to FIG. 78, method 7800 includes, at step 7825, applying adhesive to a seam formed by sensor 244 and sensor shell 7804. There may be a seam formed between the edge of the portion of the sensor shell 7804 and sensor 244. An adhesive may be applied to the seam as shown in FIG. 81. Applying an adhesive to the seam may include sealing the seam against the ingress of one or more contaminants, fluids, and portions of food or the like. Sealing the seam may include funneling saliva to the transducer of the sensor 244. In various embodiments, applying an adhesive to the seam may include applying an adhesive to the seam manually such as utilizing a toothpick or other implement with a relatively small tip to apply an adhesive to the seam area in FIG. 81. In various embodiments, applying an adhesive to the seam may include applying the adhesive automatically through one or more automated processes or robotically. Curing the adhesive between the sensor shell 5140 and the retainer 5132 may include exposing the adhesive to ultraviolet light consistent with the disclosure of UV light exposure in the entirety of this disclosure.

### 3D Printing the Retainer

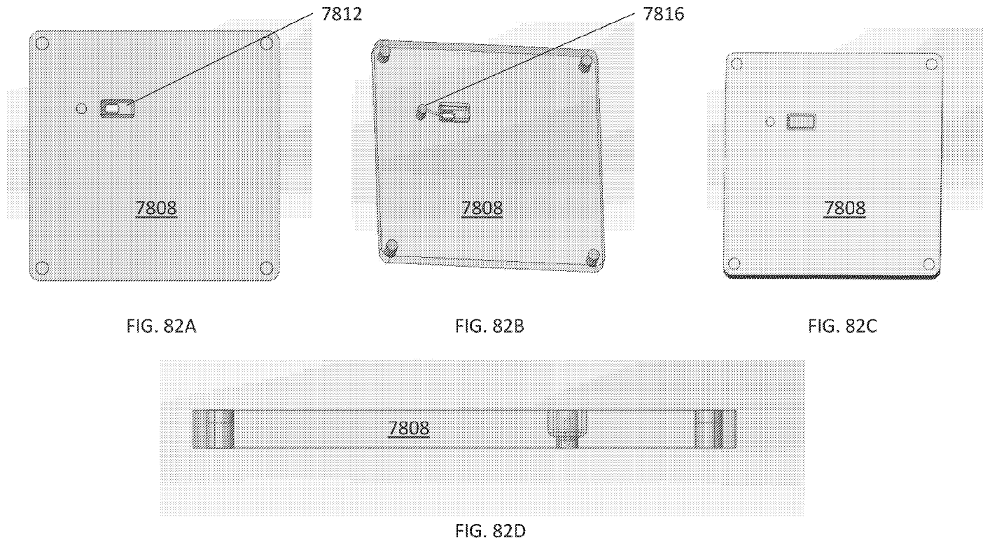

With continued reference to FIG. 78, method 7800 includes, at step 7830, providing a fixture plate 7808 with a sensor shell recess 7812 disposed therein. The fixture plate 7808 may be a generally planar plate formed from one or more plastics, metals, composites or the like. Fixture plate 7808 may be configured to couple to one or more 3D printers work areas or build plates. In various embodiments, fixture plate 7808 is a build plate of a 3D printer. In various embodiments, fixture plate 7808 includes one or more coupling features configured to fixedly attach the fixture plate to one or more components of a 3d printer, namely a build plate. For example, as shown in FIGS. 82A-82D, fixture plate 7808 may include pegs, posts, holes, threaded holes or the like configured to receive a fastener or matedly connect to a corresponding feature on the 3D printer. Fixture plate 7808 includes a sensor shell recess 7812 configured to receive and fix the sensor shell 7804 within the fixture plate 7808.

Fixture plate 7808 may include sensor shell recess 7812 corresponding in size and shape to the sensor shell 7804. Fixture plate 7808 may be configured to receive and capture sensor shell 7804 by press fit, holding sensor shell 7804 transducer-side down in fixture plate 7808, thereby exposing a portion of the backside of sensor shell 7804 to a 3D printer head.

Fixture plate 7808 may include one or more ejector pins 7816, having a first end and a second end, defining a length therebetween. The ejector pin 7816 may have a first end disposed outside of the fixture plate and a second end proximate the cavity forming sensor shell recess 7816. Ejector pin 7816 may be configured to press on sensor shell 7804 from inside the cavity, thereby ejecting sensor shell 7804 without a user needing to pull from the back side, exposing the sensor shell 7804 and retainer to harm.

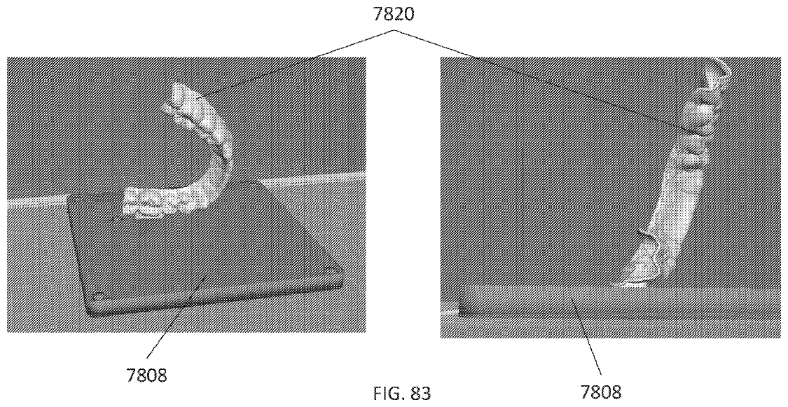

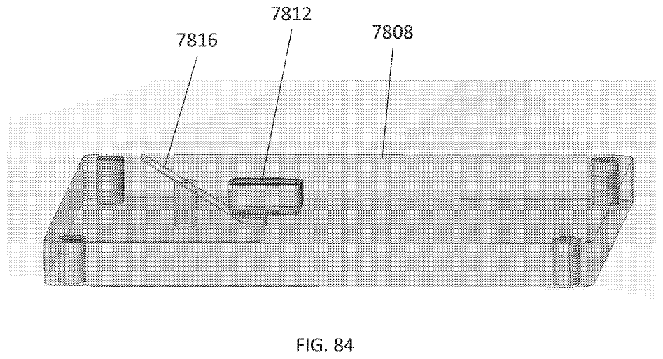

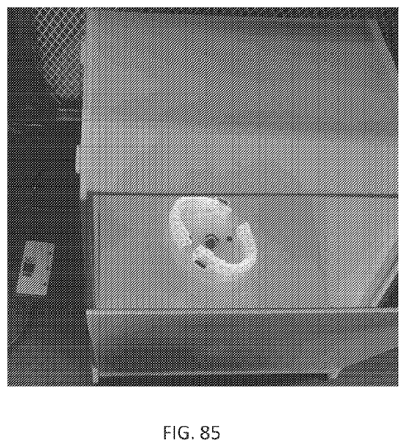

With continued reference to FIG. 78, method 7800 includes, at step 7835, additively forming a retainer 7820 on fixture the plate 7808, the retainer 7820 formed in alignment with the sensor shell 7804 and sensor 244 and coupled to the sensor shell 7804, as shown in FIG. 83. In various embodiments, retainer 7820 may be formed from one or more plastics. In various embodiments, retainer 7820 may be formed from one or more dental resins. In various embodiments a first portion of retainer 7820 may be formed from a first material as described herein and a second portion of the retainer 7820 may be formed from a second material as described herein. In various embodiments retainer 7820 may be formed from two or more types of plastic. In various embodiments, retainer 7820 may be formed from two or more types of dental resin. In various embodiments, retainer 7820 may be formed from stereolithography (SLA). In various embodiments, retainer 7820 may be formed from selective laser sintering (SLS). In various embodiments, retainer 7820 may be formed from fused deposition modeling (FDM). In various embodiments, retainer 7820 may be formed from digital light process (DLP). In various embodiments, retainer 7820 may be formed from multi jet fusion (MJF). In various embodiments, retainer 7820 may be formed from a polyjet process. In various embodiments, retainer 7820 may be formed from direct metal laser sintering (DMLS). In various embodiments, retainer 7820 may be formed from electron beam melting (EBM). In various embodiments, forming retainer 7820 includes forming a retainer model consistent with this disclosure. In various embodiments, forming retainer 7820 includes importing the retainer model. In various embodiments, forming retainer 7820 may include providing the retainer model. In various embodiments, retainer 7820 may be formed continuously and coextensively with sensor shell 7804, that is to say that the retainer 7820 may be formed from the same material and attached through deposition of said material to sensor shell 7820. In various embodiments, retainer 7820 is formed from the same material as sensor shell 7804. In various embodiments, retainer 7820 is formed from a different material than sensor shell 7804.

In various embodiments retainer 7820 is removed from the fixture plate 7808 by moving ejector pin 7816 down from the first end, thereby pushing up on the sensor shell 7804 and retainer 7820, now coupled together and forming one intraoral monitor. The ejector pin 7816 may be moved up to remove the sensor shell 7804. The ejector pin 7816 may be moved side to side or rotated to remove the sensor shell 7804, in various embodiments. In various embodiments, the ejector pin 7816 may be a distinct component and fixture plate 7808 may be configure to accept ejector pin in an opening to contact sensor shell 7804 and remove retainer 7820. In various embodiments, when a distinct ejector pin 7816 is present, the opening for receiving said pin may be plugged during printing to prevent the ingress of print material or debris during printing. In various embodiments, the fixture plate 7808 has one or more clamping features configured to hold the sensor shell 7804 or retainer 7820 at any point during the printing process. Retainer 7820 may be formed by subjecting the retainer 7820 to ultraviolet light. In various embodiments, any step of the process may include applying adhesives to seams, holes, or edges of the any component described herein to seal against ingress of contaminants or to bond components.

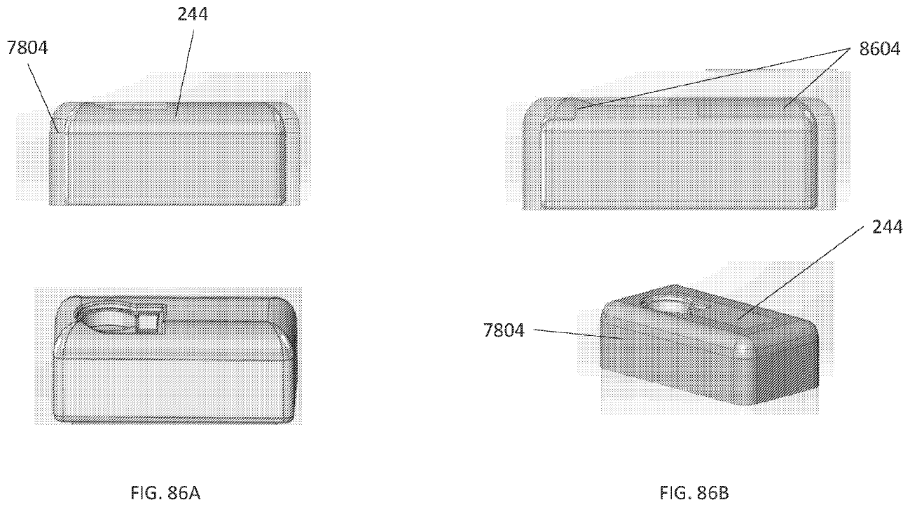

Referring now to FIGS. 86A and 86B, a sensor 244 is shown in orthogonal and elevated views, respectively. In FIG. 86A specifically, an orthogonal view of sensor 244 being partially enveloped by a retainer as described herein (shown in partial transparency is shown). In various embodiments, as shown and described, sensor 244 may include surface geometry configured to assist the retainer in gripping said sensor, such as an indented transducer side 8604, shown in FIG. 86B as projected lines through retainer. Any sensor as described herein may be used in a sensor system.

Referring now to FIGS. 87A-87B, any retainer as described herein may be configured to mount any number of teeth or any section of gum, should the user have no teeth. The retainer may be configured to mount only 1-3 teeth, as shown in FIGS. 87A-87B by partial retainer 8704. In various embodiments, a large tooth may selected to locate the sensor, the retainer configured to have the contours of said tooth and the surrounding teeth for mounting the sensor at that tooth on the buccal side of the teeth. Partial retainer 8704 may assist users who find the full retainer or a larger retainer uncomfortable, thus making long term use and therefore measurement easier for the patient or user.

The sensor system of the present invention may be seated comfortably on one (or more) tooth, such that the sensor may continually monitor one or more biomarkers (e.g., oral acidity) and transmit data to a mobile application in real time or at a pre-determined time (e.g., when a mobile device is brought within wireless communication range). In various embodiments, patients may receive notifications to monitor oral health in real-time, at the most important moments. In various embodiments, a healthcare provider (e.g., a dentist) may analyze long-term data through a web portal to provide personalized oral health strategies.

The systems of the present inventive concepts include wearable intraoral sensor devices for non-invasive measurement (e.g., continuous measurement), and these systems can provide long-term use in a patient's oral cavity, as well as wireless transmission of measurement data. In some embodiments, the sensor devices include a biosensor transducer configured for measuring pH values, analyte levels, and/or other physiologic parameters of the patient, such as for extended periods of time (e.g., at least months), and in complex oral environments. Design considerations for this sensor device can include the device's specificity and accuracy, size, sensing lifetime, biocompatibility, comfort, and/or power requirements. In some embodiments, the sensor device transduces and transmits pH and/or other physiologic parameter values, while accounting for other variables (e.g., changing variables) such as salivary flow rate, tissue contact, temperature, salivary turbidity, salivary viscosity, ionic strengths, and/or jaw movements. The accuracy of provided pH measurements can be comparable to the accuracy of conventional pH sensors (e.g., glass membrane pH sensors). In some embodiments, the transducer and other components of the sensor device are small enough to fit on the side of a tooth. In some embodiments, the sensor device is able to maintain its accuracy for a period of at least months, and it does not experience significant fouling due to mineral deposition, food deposition, and/or bacterial growth. The sensor device can be comfortable for the patient to wear long term, due to an optimized sensor-gum interface and/or sensor-cheek interface, and by ensuring the sensor device does not occlude or alter the user's bite. In some embodiments, the operational power requirements of the sensor device are small enough to be feasibly powered by a power-supplying component that can also fit onto a tooth of the patient. In some embodiments, the sensor device does not require patient action, thereby eliminating or at least reducing the potential for patient non-compliance.

One aspect of this disclosure provides a sensor device for recording, detecting, monitoring, and/or measuring (“recording”, “detecting”, “monitoring”, or “measuring” used interchangeably herein) pH, one or more analytes, temperature, another physiologic parameter, or a combination thereof. The sensor device can comprise a wearable intraoral sensor device where at least portion (e.g. a sensing portion) is positioned in an oral cavity of a patient for a time period (e.g. at least one day). The sensor device also comprises a sensor assembly, which can be coupled to the attachment mechanism, and can comprise a transducer (e.g., including one or more sensors, reference elements, and/or other sensing elements, and the associated circuitry) configured to measure one or more physiologic parameters of the patient (e.g., pH, temperature, one or more analytes, or a combination thereof), in an oral cavity or other location of the patient, and provide the measurement data in the form of a data signal (e.g., a signal suitable for transmission to a separate device).

The sensor device can include a data transmitter, operably connected to the sensor assembly, and configured to transmit the data signal (e.g., wirelessly transmit the data signal). The sensor device can include a battery, capacitor, and/or other power supply operably connected to the sensor assembly and the transmitter. In some embodiments, the sensor device further comprises a first housing, enclosing the one or more sensing elements of the sensor assembly, where the sensor assembly includes a semi-permeable membrane configured to allow desired molecules to enter one or more chambers of the sensor assembly in which sensing elements are positioned, via one or more openings in the first housing. In some embodiments, the sensor device comprises a proton exchange membrane configured to allow protons to enter the one or more chambers via the first housing. The sensor device can also comprise a second housing sealably coupled to the attachment mechanism (e.g., and surrounding at least a portion of the first housing).

In some embodiments, the sensor device comprises a first housing enclosing sensing elements within one or more chambers of the sensor device, the sensor device further including a semi-permeable membrane configured to allow desired molecules to enter the one or more chambers. In some embodiments, the sensor device comprises a first housing enclosing sensing elements within one or more chambers of the sensor device, the sensor device further comprising a proton exchange membrane configured to allow protons to enter the one or more chambers (e.g., to make physical contact with one or more of the sensing elements of the sensor device). In some embodiments, a second housing sealably couples to the attachment mechanism, and seals one or more components within the second housing (e.g., one or more sensing elements, the transmitter, and/or a power supply).

The sensor device first housing unit can define (e.g. provide walls or other surfaces to create) one or more fluid chambers for sensing electronics, such as a first chamber for a reference electrode, and a second chamber for a sensor such as an ISFET transistor. A reference electrode chamber can surround a salt solution, such as a potassium chloride (KCl) salt solution, where the salt solutions contacts the reference electrode (e.g., contact metal of the reference electrode, such as when the metal of the reference electrode comprises Ag/AgCl, and the salt solution comprising a solution or gel, “solution” herein, of saturated or super saturated KCl). The salt solution can be contained within the first chamber using a diaphragm (e.g., a ceramic diaphragm) and/or a semi-permeable membrane (e.g., a sulfonated tetrafluoroethylene-based fluoropolymer-copolymer membrane). The membrane can be configured to allow protons or desired molecules or ions to enter the first chamber. The one or more chambers can each include an opening (e.g., an opening into the second chamber) that is covered by a mesh. The mesh and the internal surface of the first and second chambers can be treated by a surface modification technique such as ion implantation and/or application of a coating (e.g., a hydrophilic coating). In some embodiments, the sensor device attachment mechanism includes any retainer as described herein

In some embodiments, the sensor device attachment mechanism comprises a dental crown (also called a “dental cap”). In other embodiments, the attachment mechanism comprises a dental implant, bridge, dentures, orthodontic temporary anchorage device (TAD), a removable dental prosthesis, and/or a removable dental appliance including, but not limited to, Herbst appliance, Activator, Bionator, twin block appliance, Pendulum appliance, Forus™ Fatigue Resistant Device, Hyrax appliance, Haas appliance, Hawley-type removable appliance with jackscrew, Quad-helix, W-arch, transpalatal arch, Nance appliance, Lower lingual arch, and/or one or more aligners (e.g., Invisalign™ teeth aligning products).

In some embodiments, the sensor devices of the present inventive concepts are configured to measure pH in an oral cavity of a patient. Periods of elevated acidity within the oral cavity can occur on the span of tens of minutes and can indicate why and/or when teeth are decaying. Existing devices are incapable of adequately (e.g., continually) measuring acidity data, such as for extended time periods, which is critical for monitoring acid attacks and preventing decay and/or crumbling of a tooth or bone, known as “caries”. Existing devices (e.g., including mouthguards, retainers, and the like) can be too large to wear in the mouth throughout the day, and require significant effort from the patient to operate or maintain (e.g., which reduces compliance and completeness of data), have short sensor lifetimes, and/or have inadequate durability to survive oral conditions.

In some embodiments, the sensor device measures one or more analytes in an oral cavity of a patient. In some embodiments, the analyte is avian influenza virus, hepatitis B marker HBsAg, cancer marker AFP, human thyroid stimulating hormone, interleukin 8 (IL-8), tumor necrosis factor (TNF-.alpha.), cancer biomarker CYFRA21-1, prostate cancer biomarker PSA, carcinoembryonic antigen (CEA), cardiac troponin I (cTnI), C-reactive protein (CRP), prostate cancer biomarker osteopontin (OPN), interleukin-6 (IL-6), cortisol, lyme disease antigen, Alzheimer biomarker amyloid-.beta., chondroitin sulfate proteoglycan 4, pancreatic cancer biomarker, carbohydrate antigen 19-9 (CA 19-9), prostate specific antigen/1-antichymotrypsin (PSA-ACT) complex, breast cancer biomarkers human epidermal growth factor receptor 2, human immunodeficiency virus (HIV), bladder cancer biomarker, urinary APOA2 protein, prostate cancer biomarker PSA-ACT complex, D-Dimer, biomarker of venous thromboembolism, breast cancer biomarker EGFR, hemoglobin-A1c, insulin, and/or other parameter as described herein. Additional analytes are described in Ana Carolina, Recent Trends in Field-Effect Transistors-Based Immunosensors, Chemosensors 2016, 4, 20, 21 Oct. 2016 (accessed here: http://www.mdpi.com: 8080/2227-9040/4/4/20/pdf), which is herein incorporated by reference.

In some embodiments, the analyte detected by the sensor device of the present inventive concepts is an analyte detectable in saliva. In some embodiments, the analyte is a hormone such as cortisol, androgens, testosterone, estriol, estrogen, progesterone, aldosterone, DHEAS. In some embodiments, the analyte is an antibody such as IgG, IgA, IgA, IgM. In some embodiments, the analyte is a growth factor such as EGF, NGF, VEGF, IGF. In some embodiments, the analyte is selected from cytokines and chemokines such as IL-1 beta, IL-8, IL-6, MCP-1, CX3CL1, GRO-1 alpha, troponin I, TNF alpha. In some embodiments, the analyte is selected from nucleic acids such as human DNA, microbial DNA, mRNA, siRNA, micro RNA (miR-125a and miR-200a). In some embodiments, the analyte is a protein detectable in saliva. In some embodiments, the analyte is a drug, including, but not limited to, drugs of abuse, ethanol, therapeutic drugs, anticonvulsants, antipyretic/analgesics, anti-neoplastic agents, anti-bacterial agents, bronchodilators, and cotinine. Additional analytes that can be measured or detected are described in Malamud D, Saliva As A Diagnostic Fluid, Dent Clin North Am. 2011 January; 55 (1): 159-78 (accessed here: https://www.ncbi.nlm.nih.gov/pubmed/21094724)

In some embodiments, the analyte detected by a sensor device of the present inventive concepts is selected from analytes that are consumed during eating and drinking. These analytes include nutritional macromolecules such as carbohydrates, proteins, and fats; allergens such as shellfish, peanuts, gluten, etc.; and toxins such as heavy metals, mercury, and the like.

In some embodiments, the sensor device measures temperature in the oral cavity of a patient. In some embodiments, the sensor device measures a combination of two or more of: pH, temperature, and/or one or more analytes in an oral cavity of a patient.

In some embodiments, a sensor device of the present inventive concepts is configured to measure pH, temperature, one or more analytes, or a combination thereof, in an oral cavity of a patient for a time period of at least one week, and/or for a time period of between one day and six months.

The sensor devices of the present inventive concepts can be constructed and arranged to take a patient's comfort into account. A patient's comfort while using the sensor device can be determined by several factors. In some embodiments, the sensor device is constructed and arranged to avoid interfering with user's occlusion (bite), speaking, and/or swallowing. In some embodiments, the sensor device is constructed and arranged to avoid protruding above the bottom molar and/or below the top molar, as this would create a noticeable interference with the user's occlusion. In some embodiments, the one or more surfaces of the sensor device is constructed and arranged to avoid discomfort to the surrounding tissues of the gum, cheek, and/or tongue. In some embodiments, the sensor device comprise a smooth outer surface, free from pockets or rough or jagged spots. In some embodiments, electronic and/or other components of the sensor device are coated with a smooth material, such as a coating which resists degradation in the mouth.

The sensing elements of this disclosure, such as those including at least an ion sensitive field effect transistor (ISFET), can be low power, small, easily and inexpensively manufactured, and accurately sense in-vivo conditions, and these sensing elements can produce an easily readable signal. The sensor assembly can comprise conditioning circuitry (also known as “signal processing” circuitry).

In some embodiments, sensing elements of the sensor device include a sensing transistor, such as an ISFET. These ISFET sensors are durable, low-power, inexpensive, and produce a current-based output signal, which can be readily amplified and processed. Additionally, ISFET sensors can be manufactured using CMOS methods, meaning they can be produced directly on an integrated circuit, in a “system on a chip” or SOC configuration. In some embodiments, the sensing assembly includes an ISFET that is fabricated to transduce the analyte ionic potential of protons in saliva to a time changing electrical current. In some embodiments, the sensing assembly further includes a reference electrode. In some embodiments, the sensing assembly includes electrodes that are made of conductive materials like gold, silver, or carbon, and the reference electrode is an Ag/AgCl paste, wire, or ink. The sensing assembly can include one or more fluid transport channels, such as a channel that is a graphene monolayer of a substrate. In some embodiments, a semi-porous hydrogel is added as a transistor gate insulator, positioned on top of semiconductor circuitry with incorporated bioreceptors that are configured to provide specificity towards a certain analyte (e.g. toward a particular physiologic parameter, or other parameter, to be measured).

In some embodiments, the sensor device of the present inventive concept comprises more than one set of analyte sensors. For example, the sensor device can comprise more than one ISFET sensor, wherein each ISFET sensor is configured to measure a different parameter (e.g., pH, temperature, or an analyte). In some embodiments, each ISFET sensor is contained in a chamber (e.g. as defined by walls of a housing), wherein an opening to the chamber is covered by a semi-permeable membrane configured to allow detection of a certain variable by the ISFET sensor. In some embodiments, the ISFET sensor is installed on a substrate, such as a supporting printed circuit board (PCB). In some embodiments, two or more additional ISFET sensors are added to the PCB or other substrate (“PCB” or “substrate” herein) to enable a multiplicity of molecules to be measured. Each ISFET can include a gate that includes a functionalized layer with bioactive elements and/or elements exhibiting preferred binding specificity to a particular analyte of interest. Generally, the membranes described herein (e.g. a membrane covering an ISFET) can comprise a chemically reactive membrane, or other membrane modified by adding inorganic and/or organic chemical or biological receptors such as antibodies, aptamers, and/or small molecules, depending on the analyte to be detected or measured. The selective membrane can enable the sensor device to measure, for example, Na+, Ca2+, proteins, carbohydrates, fats, allergens, toxins, and other analytes described herein. Further, each ISFET can be positioned in a chamber (e.g. each in a unique chamber) with an opening to the chamber covered by a membrane or filter (e.g. “membrane” herein, each chamber comprising a similar or different membrane). For example, a covering membrane can be configured to selectively or semi-selectively conduct specific molecules into the sensing chamber, which can enable the sensor device to measure, for example, Na+, Ca2+, proteins, carbohydrates, fats, allergens, toxins, and other analytes described herein. The ability to simultaneously detect a multiplicity of unique analytes may be accomplished by creating an array of functionalized ISFET sensors bonded to conductive pads plated with gold, silver, platinum, etc., and may be commonly serviced with one or more reference electrodes also operating from one or more PCB pads. These pads can be connected to signal processing circuitry using copper (or other) PCB traces which, can be routed on the top, bottom or through one or more layers of the PCB.

In some embodiments, the sensing elements can be encased in a protective housing. In some embodiments, an opening into a chamber comprising a sensing element (e.g. an ISFET and/or reference electrode) is covered by a mesh, a proton exchange membrane, and/or a diaphragm (e.g. a ceramic diaphragm), which can be constructed and arranged to allow fluids, protons, and/or electrolytes to diffuse through the covering to the associated chamber, and to limit the diffusion of larger analytes and undesired particles. The covering can protect the sensing elements within the associated chamber from physical and/or chemical factors in the mouth. In some embodiments, the sensing elements (e.g. electrodes and/or other portions of the sensing elements) are patterned using methods including screen-printing, photolithography, evaporation, electroplating, and/or physical vapor deposition (sputtering). In some embodiments, the semiconducting material of the ISFET can be made using graphene, which can be fabricated using chemical vapor deposition (e.g. a deposition on copper).

In some embodiments, a sensing element is an ISFET, such as an ISFET including a conductive or semi-conductive channel (e.g. gate) material (e.g. graphene), to transduce the ionic potential (e.g. pH) surrounding the ISFET to an electrical signal that can be transmitted.

In some embodiments, the sensor devices of the present inventive concepts measure pH in the oral cavity. In certain embodiments, the sensor device measures pH of saliva in the oral cavity. The pH of saliva can be influenced by various oral environmental factors, including, but not limited to, saliva, biofilm (tooth plaque), intrinsic and extrinsic oral fluids (including vapor), food, and breath.

In some embodiments, a sensing element comprising an ISFET is modified to detect multiple types of molecules in the oral cavity. In some embodiments, the functional groups of an ISFET sensor are modified. Generally, the membranes described herein can be modified by adding inorganic or organic chemical or biological receptors such as antibodies, aptamers, and/or small molecules, depending on the analyte to be detected or measured. Additional analytes are described in Ana Carolina, Recent Trends in Field-Effect Transistors-Based Immunosensors, Chemosensors 2016, 4, 20, 21 Oct. 2016 (accessed here: http://www.mdpi.com:8080/2227-9040/4/4/20/pdf), which is herein incorporated by reference. Additional ISFET modifications are described at Torsi, Organic field-effect transistors sensors: a tutorial review, Chem Soc Rev., 21 Nov. 2013; 42(22):8612-28 (accessed here: https://www.ncbi.nlm.nih.gov/pubmed/24018860); and Lerner, Detecting Lyme disease using antibody-functionalized single-walled carbon nanotube transistors, Biosens Bioelectron. 15 Jul. 2013; 45:163-7 (accessed here https://www.ncbi.nlm.nih.gov/pubmed/23475141) which are incorporated by reference herein.

The sensor devices of the present inventive concepts collect and transmit data reliably. The sensor assemblies can create reliable data with a two-prong approach. First, in static fluid settings, the output of the sensor assembly consistently matches with a given pH value. This consistency can be achieved by testing the sensor assembly with various solutions with known pH values, and then creating a calibration curve. The pH of unknown solutions can then be determined by measuring the sensor assembly output, and then comparing this output to the measured pH value of the solution. Solutions with similar chemical and physical consistencies to saliva can be included in the testing. The second prong is determining whether the sensor assembly is able to measure pH accurately in the dynamic conditions of the mouth. The sensor devices of the present inventive concepts can account for the dynamic conditions of the mouth, including different salivary flow rates, turbidity, and viscosity, different contact from surrounding tissue like cheeks or tongue, as well as talking, yawning, chewing, and swallowing. In some embodiments, the reliability of the sensor device's data is confirmed, during testing, in a simulated mouth environment. Through this two-prong approach, the sensor devices of the present inventive concepts can provide consistently reliable intraoral pH and other measurements.

The sensor devices of the present inventive concepts can be configured to wirelessly transmit data. Multiple modes of data transmission can be used. The modes of transmission can be passive to the user (e.g. the patient, a family member of the patient, and/or a clinician of the patient), requiring little to no time commitment beyond initial installation of the sensing device to receive data. The modes of transmission can require little to no time commitment for charging or cleaning of the sensor device. The modes of transmission can also avoid requiring an intermediate receiver, which would force users to remember to wear the intermediate receiver, charge it, clean it, etc. In embodiments with an active circuit, energy can be provided by a chemical battery (silver oxide, nickel hydride, lithium polymer, lithium ion, and/or zinc oxide batteries), such as to power a FET, amplify the signal from the FET, and transmit it to a smart device or intermediate receiver, such as a transmission performed via BLE (Bluetooth low energy), WLAN, Wi-Fi or ZigBee or another wireless communication technology described herein. In some embodiments, a transistor signal is sent through analog front end (AFE), to Balun, antenna, and BLE components. Some embodiments comprise a similar electronic pathway with low pass filters to receive more stable readings.

In some embodiments, the sensor devices of the present inventive concepts wirelessly transmits data via Bluetooth technology. In some embodiments, the sensor device wirelessly transmits via a wireless local area network (WLAN), Wi-Fi (wireless fidelity), ZigBee, near-field communication (NFC), ANT, Thread, Zigbee, WiMAX, WWAN, MANET, PAN, Wireless Hart, Z-Wave, MESH, UWB, IrDA, Cellular, Peer-To-Peer, and 802.11 variants. In some embodiments, the sensor device wirelessly transmits via frequencies ranging from sub-sonic to ultraviolet. In some embodiments, the wearable oral sensor wirelessly transmits using modulation methods including, but not limited to, OOK, AM, FM, SSB, FSK, PSK, GFSK, and MSK. In some embodiments, the sensor device wirelessly transmits using Near Field, Mid Field, and/or Far Field magnetic and/or electric field radiation.

The tissue penetration profile of the signals wirelessly transmitted by the sensor device may be determined by passing the signal through real animal tissue or simulated tissue with varying thickness.

In some embodiments, any retainer as disclosed herein may include or be manufactured to contain an antenna. This antenna configuration is accomplished through use of an impedance matching circuit from the SOC's radio transceiver to the metallic or semi-metallic structure of the molar band. Employing RF instrumentation such as a Vector Network Analyzer (VNA), the components comprising the impedance matching circuit can be adjusted in combination to cause resonance at the frequency of interest and thereby provide the optimal transfer of RF energy either flowing outwards towards the antenna or inwards toward the RF transceiver. This adjustment process (tuning) can be performed in situ, and it can be facilitated through use of external RF instrumentation which measures the radiated field strength from the transmitting device. In various embodiments, metallic material near the antenna may affect antenna performance. In various embodiments, the device may include a non-metallic portion near (e.g., directly above) the antenna. In various embodiments, the non-metallic portion may include a polymer. In various embodiments, the non-metallic portion may be a UV-curable epoxy.

In various embodiments, the casing of the device may be used as the antenna. In various embodiments, the casing includes a metal or combinations of metals (e.g., an alloy). In various embodiments, the printed circuit contains no component antenna, but instead the pads for the Radio Frequency (RF) line may be soldered either directly or via a wire to the inside of the casing. In various embodiments, oscillating voltage is transferred to the casing which produces the electric field. In various embodiments, the inside of the casing where the PCB connection is soldered may be chemically plated with a metal which may be easily soldered to (e.g., nickel, tin, copper, etc.).

In various embodiments, the case may include an antenna cutout and/or waveguide. In various embodiments, as described above, no metal may be disposed near the antenna. In various embodiments, the portion of the case for the antenna may be a hole. In various embodiments, the antenna may be seated such that the antenna is directly pressed against the inside of the casing and into the hole. In various embodiments, the hole may function as a waveguide by directing electromagnetic radiation from the antenna in a specific direction, increasing the intensity in said direction (e.g., towards the cheek).

In some embodiments, a sensor device of the present inventive concepts wirelessly transmits a signal comprising measurement data at intermittent intervals. In some embodiments, the sensor device transmits the data signal on a regular basis, such as once per minute, once every five minutes, once every ten minutes, and/or repeated at intervals between once per minute and once per 30 minutes. In some embodiments, the sensor device wirelessly transmits the data signal immediately after the data signal is generated.

In some embodiments, a second device of the present inventive concepts wirelessly transmits at different intervals depending on measurement time resolution and battery life. These intervals can range from immediate (e.g., once per millisecond) to long term (e.g., once per year), depending upon patient need(s) and the molecule(s) being measured. In some embodiments, in balancing measurement time resolution against battery life, sensor device measurement data is stored in memory and then transmitted together (e.g., in multiple) as a single packet. In some embodiments, in balancing measurement time resolution against battery life, sensor device data is conditionally wirelessly transmitted depending on when and/or how often measurement data deviate from predetermined and/or predicted values.

In some embodiments, the sensor device stores data (e.g., all measured data) in embedded memory and only transfers the data (e.g., all or a portion of the data) through a connection event, such as a connection event that is initiated automatically by the system and/or manually by the user.

The sensor devices of the present inventive concepts can comprise a power source (e.g., a battery and/or a capacitor). The described modes of data transmission can be configured to work with a different powering mode. In embodiments with active circuits, power can be delivered by an onboard battery to supply a source drain voltage and source gate voltage over a FET, amplify the signal, and transmit it.

The sensor devices of the present inventive concepts can be constructed and arranged to withstand damage from a variety of environmental factors, including damage via physical shear forces, damage via chemical corrosion, and/or damage from formation of a biofilm or food layer over the sensing surface in the complex intraoral environment. In some embodiments, the sensing assembly of the sensing device comprises a proton exchange membrane. In such embodiments, the sensing elements (e.g., the ISFET and/or reference electrode) is exposed to salivary analytes only through a proton exchange membrane. The protons must diffuse across the membrane in order to reach the one or more sensing elements. In some embodiments, the sensing element(s) is enclosed in a protective housing which protects the transducer from physical and other forces experienced in the mouth, prolonging sensing lifetime, and reducing noise.

In some embodiments, one or more surfaces (e.g., one or more outer surfaces) of a sensor device of the present inventive concepts is made of an antifouling material that resists biofilm deposition, prolonging the lifetime of the sensor device. In some embodiments, all or a portion of the sensor device outer surface is chemically treated with antibiotics and/or hydrophobic compounds, such as to prevent materials from adhering. In some embodiments, the sensor device comprises an antimicrobial peptide coating. In some embodiments, the all or a portion of the outer surface of the sensor device is smooth, such as with no pockets, jagged edges, gaps, or overhangs in which debris or bacteria can collect. In some embodiments, a physical antifouling surface (e.g., a geometric pattern) is provided on the outside of the sensor device, such as a pattern similar to sharkskin. In various embodiments, the sensor may be protected from biofouling using methods that are known in the art. In various embodiments, if biofouling occurs, any debris can be removed by brushing the device similar to how a tooth would be brushed, or letting the device soak in a cleaning solution such as a denture cleaner.

In some embodiments, a sensor device of the present inventive concepts comprises a biocompatible epoxy which is ISO-10993-4,5,6,10,11 approved. Such a coating can reduce irritation or inflammation of tissue surrounding the sensor in the oral cavity.

### Methods of Installation

In some embodiments, the sensor devices of the present inventive concepts are designed to be installed in the mouth of the patient by a dental provider (e.g. a dentist and/or an oral hygienist).

In some embodiments, the method further comprises syncing the sensor device to an application installed on a receiving device. The method can further comprise adding identifying information of the patient to the application.

### Methods for Measuring pH, Temperature, and Analytes in Patients

The sensor devices of the present inventive concepts can be worn by healthy mammals and/or mammals with various health conditions (either referred to as “patient” herein). Patients who have many cavities and very poor oral health could greatly benefit from using these sensor devices. In some embodiments, the patient is informed throughout the day when their oral pH is approaching critical levels and taught to correct the imbalance in real time. In some embodiments, when a patient receives a notification of low oral pH, he or she will also receive recommendations for how to correct the imbalance in the mobile application (pH correcting mouthwash, pH correcting oral spray, brushing teeth, etc.). Furthermore, in some embodiments, a clinician (e.g. a doctor or other health care provider) of the patient can track the data to identify trends in oral pH levels and better diagnose the source of the disease. If a patient's pH drops below a threshold (e.g. 5.5) at night, caries could be occurring due to xerostomia or conditions that occur while sleeping. If the patient's pH drops after meals, the clinician could recommend different dietary habits. The sensor device can also be used to track the effectiveness of one or more particular treatments, in between dental and/or other clinical visits. The lifespan of permanent restorations, like crowns, root canals and implants will increase significantly if oral pH is maintained at a healthy level. In this way, insurance companies will save on costs associated with re-treatment and dentists will be able to avoid misplaced blame for failing treatment. Individuals with mild and good oral health could also benefit from using this sensor in similar ways. They can be notified (e.g. in real time) whenever oral pH drops below a threshold (e.g. a critical level) and effectively prevent the onset of caries and receive recommendations in the mobile application on how to correct the oral pH (e.g. via mouthwash, brushing teeth, and/or other corrective measures) before carious infections develop. This corrective action can save a great amount of pain and suffering from experiencing cavities, reduce money spent on treating carious infections, and reduce time spent at dentists' offices. If caries does begin to occur, a clinician can monitor the pH levels over time to better assess their origin and provide more personalized and effective treatments.

Another aspect of this disclosure is directed to a method for continually and/or intermittently measuring pH, temperature, one or more analytes, or a combination thereof, in an oral cavity of a patient. The method comprises installing a wearable intraoral sensor of this disclosure in the oral cavity of the patient. This disclosure contemplates wearable intraoral sensors that can be inexpensively and quickly installed.

In some embodiments, the sensor housing comprises one or more sensing elements (e.g. an ISFET and/or a reference electrode), a controller, and a power source. A proton exchange membrane can be included to allow passage of specific materials only (avoiding non-desired materials) to chambers including the sensing elements. A sensor assembly can be connected to an attachment mechanism (e.g. including a band) with a seal. In some embodiments, saliva directly contacts the membrane of the sensor device.

In some embodiments, a sensor device of the present inventive concepts is configured to continually and/or intermittently measure pH in the oral cavity for at least one day, at least one week, at least one month, or at least 3 months.

The methods of the present inventive concepts can also comprise measuring pH, temperature, one or more analytes, or a combination thereof, in the oral cavity of the patient for at least one day, thereby generating measurement data. In some embodiments, the measurement data is pH measurement data, temperature measurement data, analyte measurement data, or a combination thereof.

The methods of the present inventive concepts can also comprise wirelessly transmitting the measurement data from the sensor device at intermittent intervals. In certain embodiments, the measurement data is wirelessly transmitted once per minute, once per five minutes, once per ten minutes, and/or at an interval between once a minute and once every 30 minutes. In some embodiments, the measurement data is wirelessly transmitted immediately after the data signal is generated. In some embodiments, measured data is transmitted when requested by the receiving device at variable, non-defined intervals.

In some embodiments, the methods of the present inventive concepts include wirelessly transmitting the measurement data from the one or more sensors at intermittent intervals to a receiving device. In some embodiments, the receiving device is a smart device. In certain embodiments, the smart device is a smart phone, a smart watch, a tablet, a smart home device (e.g., an Amazon Echo™ device or other device using Amazon Alexa™ technology, a Google Home™ device, or a smart device manufactured by Apple), and/or a computer. In some embodiments, the smart device is an iPhone or Android phone. In some embodiments, the method further comprises configuring the receiving device and/or smart device to receive the measurement data. In some embodiments, configuring a receiving device or smart device to receive the measurement data comprises installing an application on the receiving device or smart device.

In some embodiments, the methods of the present inventive concepts include wirelessly transmitting measurement data to Apple, Android, Nokia, and/or other smartphones, such as a transmission comprising Bluetooth, WiFi and/or Near Field communications capabilities. In some embodiments, the receiving device is a laptop, PC, and/or other smart device equipped with Bluetooth, WiFi, and/or Near Field communications capabilities. In some embodiments, the receiving device acts as a “relay station” and sends measurement data to the Internet, the “Cloud,” and/or to another computer system. In some embodiments, the receiving device is an Apple, Google, or Amazon “Smart Home” device (e.g., a Siri-enabled, Google Home-enabled, or Alexa-enabled device) which, in addition to functioning as a relay station, can interact with the patient in which the sensing device is installed (e.g. in the mouth of the patient).

In some embodiments, the receiving device is a custom product specifically designed for use with the wearable intraoral device. The custom device can provide for a variety of audio, visual, and/or haptic components configured for interaction with the patient. These components can include, but are not limited to, LEDs, speakers, vibrators, and text or graphic displays.

Existing devices and methods do not display patient data measured in the patient's oral cavity. In some embodiments, an application of the present inventive concepts is configured to display measurement data on a display of the receiving device. In some embodiments, the application is configured to display measurement data in graphical form on a display of the receiving device. In some embodiments, the application is configured to display measurement data over time in graphical form on a display of the receiving device. In some embodiments, the application is configured to display pH or other measurement data over time in graphical form with an indication of a critical value (e.g. a critical pH value) on the display of the receiving device.

In some embodiments, the methods of the present inventive concepts comprise transmitting patient data from a receiving device and/or smart device to a medical office, such as a dental office. In some embodiments, the method further comprises transmitting patient data from a receiving device and/or smart device to a research institution and/or a corporation (e.g. a manufacturer of the system of the present inventive concepts), such as to perform data processing or other function. In some embodiments, the method comprises syncing data between the sensor device and the receiving device and/or smart device.

In some embodiments, the methods of the present inventive concepts include orienting a housing of the sensor device buccally to measure pH, temperature, one or more analytes, or a combination thereof, from saliva gathered in the cheek of a patient. In some embodiments, the method comprises orienting the transducer proton exchange membrane buccally.

In various embodiments, during fabrication, a curable polymer (e.g., UV-curable epoxy) may be applied to any of the components described herein to thereby secure that component within its respective housing. For example, the interproximal connector may be coated with UV-curable epoxy and affixed to a metal band. In various embodiments, the curable polymer may be used to fix a flexible component (e.g., the interproximal connector) in a fixed position or shape. In various embodiments, for metallic components, laser welding may be used to connect components, seal components, or otherwise join metallic components during fabrication. In various embodiments, any gaps along the interproximal connecter may be filled with a potting agent and cured.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the inventive concepts, which is defined in the accompanying claims.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method for forming an intraoral monitor, the method comprising:
providing a first base mold corresponding in shape to at least a portion of a set of teeth;
providing a second base mold, the second base mold corresponding to the set of teeth and a sensor;
thermoforming an inner retainer around the first base mold, the inner retainer configured to abut at least a portion of a buccal side of the set of teeth;
thermoforming an outer retainer around the second base mold, the outer retainer configured to mount at least a portion of the set of teeth wherein, the outer retainer comprises:
an inner surface abutting the set of teeth and an outer surface comprising:
occlusal side, a buccal side and a lingual side; and
a pocket disposed on the buccal side, the pocket having an opening formed on the inner surface;
positioning the sensor in the pocket;
coupling the outer retainer to the inner retainer; and
removing a portion of the outer retainer proximate the sensor to form an opening in the outer retainer.

2. The method of claim 1, wherein forming the inner retainer comprises trimming a perimeter portion of the inner retainer.

3. The method of claim 1, wherein forming the outer retainer comprises trimming a perimeter of the outer retainer.

4. The method of claim 1, wherein positioning the sensor in the pocket comprises applying an adhesive to the sensor.

5. The method of claim 4, wherein positioning the sensor in the pocket further comprises applying an adhesive to an exposed inner side of after the sensor is positioned in the pocket.

6. The method of claim 1, wherein coupling the outer retainer to the inner retainer includes curing the adhesive via exposure to ultraviolet light.

7. The method of claim 1, wherein removing a portion of the outer retainer proximate the sensor includes cutting a portion of the outer retainer away from the sensor, thereby exposing a transducer disposed within the sensor.

8. The method of claim 1, further comprising applying an adhesive to a seam formed between the outer retainer and the sensor.

9. The method of claim 1, wherein coupling the outer retainer to the inner retainer comprises exposing an overlapping area formed by the outer retainer and the inner retainer to a laser beam.

10. The method of claim 1, wherein the sensor is positioned adjacent to a single tooth.

11. A method for forming an intraoral monitor, the method comprising:
providing an inner thermoform mold corresponding in shape to at least a portion of a set of teeth;
providing an outer thermoform mold corresponding in shape to at least a portion of the set of teeth and the contours of a sensor protrusion formed thereon;
thermoforming an inner retainer around the inner thermoform mold, the inner retainer configured to mount at least a portion of the set of teeth wherein the inner retainer comprises
an inner surface configured to abut the set of teeth and an outer surface;
thermoforming an outer retainer around the outer thermoform mold, the outer retainer having a pocket formed by the protrusion;
positioning a sensor in the pocket;
applying an adhesive to an exposed portion of the sensor once positioned in the pocket;
coupling the outer retainer and sensor to the inner retainer, thereby closing the pocket;
removing a portion of the outer retainer proximate the sensor to form an opening in the outer retainer.

12. The method of claim 11, wherein forming the inner retainer comprises trimming a perimeter portion of the inner retainer.

13. The method of claim 11, wherein, forming the outer retainer comprises trimming a perimeter of the outer retainer.

14. The method of claim 11, wherein positioning the sensor in the pocket comprises applying an adhesive to the sensor.

15. The method of claim 11, wherein coupling the outer retainer to the inner retainer comprises curing the adhesive via exposure to ultraviolet light.

16. The method of claim 11, wherein removing a portion of the outer retainer proximate the sensor comprises cutting a portion of the outer retainer away from the sensor, thereby exposing a transducer disposed within the sensor.

17. The method of claim 11, further comprising applying an adhesive to a seam formed between the outer retainer and the sensor.

18. The method of claim 11, wherein coupling the outer retainer to the inner retainer comprises exposing an overlapping area formed by the outer retainer and the inner retainer to a laser beam.

19. The method of claim 11, wherein the sensor is positioned adjacent to a single tooth.

20. A method for forming an intraoral monitor, the method comprising:
providing a contour model, the contour model defining an outer mold line of at least a portion of a set of teeth;
additively forming the retainer based on the contour model, the retainer configured to mount onto at least a portion of the set of teeth, the retainer comprising an inner surface configured to abut at least a portion of the set of teeth and an outer surface, the outer surface comprising:
a buccal side, an occlusal side and a lingual side;
wherein forming the retainer comprises forming a sensor recess coextensive with the outer surface;
forming a sensor shell, the sensor shell comprising a mounting side configured to mount on the sensor recess, the sensor shell further comprising an opening disposed opposite the mounting side;
applying an adhesive to a sensor and the sensor shell;
coupling the sensor within the sensor shell, the sensor comprising a transducer side proximate the opening in the sensor shell;
coupling the mounting side of the sensor shell to the sensor recess.

21. The method of claim 20, wherein the sensor shell comprises a top wall, a bottom wall, left wall and right wall, each disposed perpendicularly to the mounting side.

22. The method of claim 21, wherein the sensor recess comprises a top wall, a bottom wall, a left wall and a right wall corresponding with the sensor shell.

23. The method of claim 22, wherein applying an adhesive to the sensor recess comprises applying an adhesive to the top, bottom, left and right walls.

24. The method of claim 20, wherein coupling the sensor to the sensor shell comprises curing the adhesive via exposure to ultraviolet light.

25. The method of claim 20, wherein coupling the sensor shell to the retainer comprises curing the adhesive via exposure to ultraviolet light.

26. The method of claim 20, further comprising applying an adhesive to a seam formed between the sensor shell and the sensor.

27. The method of claim 20, further comprising subjecting the retainer and retainer shell to ultraviolet light.

28. The method of claim 20, wherein the sensor is positioned adjacent to a single tooth.

29. A method for forming an intraoral monitor, the method comprising:
providing a sensor;
forming a sensor shell, the sensor shell configured to mount onto the sensor and wherein the sensor shell further comprises a mounting side, a transducer opening side and at least one sidewall therebetween;
applying an adhesive to the sensor and/or the at least one sidewall;
coupling the sensor to the sensor shell;
applying an adhesive to a seam formed by the sensor and/or the transducer opening side of the sensor shell;
providing a fixture plate, the fixture plate comprising,
a sensor shell recess disposed thereon and configured to receive the sensor shell and
an ejector pin having a first end disposed underneath the sensor shell recess and a second end disposed above the fixture plate, defining a member therebetween;
additively forming a retainer on the fixture the plate, the retainer formed in alignment with the sensor shell and sensor and attached to the sensor shell.

30. The method of claim 29, wherein the sensor is positioned adjacent to a single tooth.

31. The method of claim 29, wherein forming the retainer comprises forming the retainer coextensively with the sensor shell.

32. The method of claim 29, further comprising removing the retainer from the fixture plate by moving the ejector pin to contact the sensor shell.

33. The method of claim 29, wherein the fixture plate is removably attached to a build plate within a 3D printer.

34. The method of claim 29, wherein the sensor shell is formed from the same material as the retainer.

35. The method of claim 29, further comprising the retainer and sensor shell are subjected to ultraviolet light.

36. The method of claim 29, wherein the sensor comprises at least two planar surfaces, each planar surface adjoining an adjacent planar surface at a rounded edge portion.
